(19) United States
(12) Patent Application Publication
(10) Pub. No.: US 2026/0144846 A1
(43) Pub. Date: May 28, 2026

(54) Modulation Of Tumor Immunity By Protein-Mediated O2 Delivery

(71) Applicant: Omniox, Inc., San Francisco, CA (US)

(72) Inventors: Stephen P.L. CARY, San Mateo, CA (US); Ana KRTOLICA, San Francisco, CA (US); Natacha LE MOAN, San Francisco, CA (US); Jonathan A. WINGER, Oakland, CA (US); Kevin G. LEONG, Millbrae, CA (US)

(21) Appl. No.: 19/188,574

(22) Filed: Apr. 24, 2025

Related U.S. Application Data
(63) Continuation of application No. 17/547,748, filed on Dec. 10, 2021, now Pat. No. 12,285,463.
Application No. 17/547,748 is a continuation of application No. 15/558,957, now abandoned.
(60) Provisional application No. 62/134,543, filed on Mar. 17, 2015.

(51) Int. Cl. A61K 38/16; A61K 39/39; A61K 45/06; A61K 47/60; A61N 5/10; A61P 35/00

(52) U.S. Cl. CPC A61K 38/164; A61K 39/39; A61K 45/06; A61K 47/60; A61P 35/00; A61N 5/10; A61N 2005/1098

(57) ABSTRACT

The invention provides methods to modulate hypoxia-mediated tumor immunity by administration of an O₂ carrier polypeptide (eg., an H-NOX protein). The methods of the invention target both hypoxia inducible factor 1 alpha (HIF-1α) pathways and non-HIF-1α pathways of tumor immunity. Such methods are useful in the treatment of a wide variety of cancers and may be used alone or in combination with other anti-cancer therapies.

### CROSS-REFERENCE TO RELATED APPLICATIONS

This application is a continuation of U.S. patent application Ser. No. 17/547,748, filed Dec. 10, 2021, which claims the benefit of U.S. Provisional Patent Application No. 62/134,523, filed Mar. 17, 2015, the disclosure of which is hereby incorporated by reference in its entirety for all purposes.

### INCORPORATION OF THE SEQUENCE LISTING

This application contains a Sequence Listing, which is hereby incorporated herein by reference in its entirety. The accompanying Sequence Listing xml file, named “ST26 051876-505C02US.xml”, was created on Feb. 6, 2026 and is 39,647 bytes.

### TECHNICAL FIELD

This application pertains to the modulation of tumor immunity by delivering oxygen to the tumor by way of a protein O₂ carrier polypeptide; for example an H-NOX protein.

### BACKGROUND OF THE INVENTION

The hypoxic tumor microenvironment suppresses the host's immune anti-tumor defenses by modulating multiple signaling pathways including, but not limited to, hypoxia inducible factor (HIF-1) signaling (Codo et al., 2014 Oncotarget, 5 (17), 7651-7662; Lee, Mace, & Repasky, 2010 Int J Hyperthermia, 26 (3), 232-246; Wei et al., 2011 PLoS One, 6 (1), e16195). Major hypoxia immunomodulating pathways are summarized in FIG. 1. Briefly, HIF-1 has been shown to: a) activate adenosinergic A2 and PD-L1 pathways that inhibit recruitment and activation of helper and killer T-cells and NK cells, key effectors of anti-tumor responses (Noman et al., 2014 J Exp Med, 211 (5), 781-790; Ohta et al., 2006 Proc Natl Acad Sci USA, 103 (35), 13132-13137); b) recruit and activate inhibitory regulatory T cells (Treg), tumor associated macrophages (TAM) and other myeloid-derived suppressor cells (MDSC) (Chaturvedi et al., 2014 Proc Natl Acad Sci USA, 111 (20), E2120-2129; Corzo et al., 2010 J Exp Med, 207 (11), 2439-2453; Wei et al., 2011); c) directly inhibit the ability of tumor cells to be recognized by immune system (Siemens et al., 2008 Cancer Res, 68 (12), 4746-4753). In addition, HIF-1-dependent and -independent epigenetic mechanisms contribute to inhibition of anti-tumor immune-responses and enhance tumor growth, angiogenesis and metastasis (Codo et al., 2014; Mimura et al., 2011 J Pharmacol Sci, 115 (4), 453-458).

In mouse metastatic tumor models, continuous supplemental oxygenation has been shown to inhibit tumor growth and prevent tumor's immune escape through inhibition of A2AR (A2A adenosine receptor) adenosinergic pathway leading to T and NK cell activation (Hatfield et al., 2015 Sci Transl Med, 7 (277), 277ra230). Specifically, continuous treatment with 60% respiratory oxygen of mice bearing MCA205, B16 or 4T1 pulmonary metastases resulted in >2-fold decrease in number of metastatic foci and enhanced survival. These data correlated with decrease in tumor and lymphocyte hypoxia, increased activated CD8 T cell (CD8+CD69+CD44+) tumor infiltration, upregulation of immunostimulating cytokines and chemokines and were dependent on intact A2AR signaling. At the same time respiratory hyperoxia was shown to reduce the number and suppressive the activity of Treg in pulmonary tumor microenvironment (TME) due to reduced Foxp3, CD39/CD73 (adenosine generating enzymes upstream of A2AR) and CTLA-4 expression. Finally, tumor regression induced by dual CTLA-4/PD-1 blockade of pulmonary tumors was enhanced by continuous respiratory hyperoxia.

Despite convincing pre-clinical evidence demonstrating the capacity of tumor oxygenation to reverse immunosuppressive TME and inhibit tumor growth, in human clinical trials supplemental oxygenation using hyperbaric or normobaric oxygen yielded limited effects (Overgaard, 2007 J Clin Oncol, 25 (26), 4066-4074). This is likely due to the inability of soluble oxygen to effectively diffuse beyond ˜80 μm from blood vessels, limiting its penetration deep into hypoxic tumor tissue. Therefore, the need exists for oxygen delivery agents that penetrate into patients' tumors to transport oxygen beyond the normal diffusion limits, and thereby oxygenate hypoxic microenvironments to impede immunosuppressive pathways. This will result in maximal stimulation of anti-tumor immune responses, both alone and in combination with other immune checkpoint inhibitors and other cancer immunotherapy approaches.

H-NOX proteins (named for Heme-Nitric oxide and OXygen binding domain) are members of a highly-conserved, well-characterized family of hemoproteins (Iyer, L M et al. (2003) BMC Genomics 4 (1): 5; Karow, D S et al. (2004) Biochemistry 43 (31): 10203-10211; Boon, E M et al. (2005) Nature Chem. Biol. 1:53-59; Boon, E M et al. (2005) Curr. Opin. Chem. Biol. 9 (5): 441-446; Boon, E M et al. (2005) J. Inorg. Biochem. 99 (4): 892-902; Cary, S P et al. (2005) Proc Natl Acad Sci USA 102 (37): 13064-9; Karow D S et al. (2005) Biochemistry 44 (49): 16266-74; Cary, S P et al. (2006) Trends Biochem Sci 31 (4): 231-9; Boon, E M et al. (2006) J Biol Chem 281 (31): 21892-902; Winger, J A et al. (2007) J Biol Chem. 282 (2): 897-907). H-NOX proteins are nitric-oxide-neutral, unlike previous hemoglobin-based oxygen carriers, H-NOX do not scavenge circulating nitric oxide (NO), and thus are not associated with hypertensive or renal side effects. The intrinsic low NO reactivity (and high NO stability) makes wild-type and mutant H-NOX proteins desirable blood substitutes because of the lower probability of inactivation of H-NOX proteins by endogenous NO and the lower probability of scavenging of endogenous NO by H-NOX proteins. Importantly, the presence of a distal pocket tyrosine in some H-NOX proteins (Pellicena, P. et al. (2004) Proc Natl. Acad Sci USA 101 (35): 12854-12859) is suggestive of undesirable, high NO reactivity, contraindicating use as a blood substitute. For example, by analogy, a Mycobacterium tuberculosis hemoglobin protein, with a structurally analogous distal pocket tyrosine, reacts extremely rapidly with NO, and is used by the Mycobacterium to effectively scavenge and avoid defensive NO produced by an infected host (Ouellet, H. et al. (2002) Proc. Natl. Acad. Sci. USA 99 (9): 5902-5907). However, it was surprisingly discovered that H-NOX proteins actually have a much lower NO reactivity than that of hemoglobin making their use as blood substitutes possible.

H-NOX proteins for the delivery of O₂ and/or NO for therapeutic and other uses are described in U.S. Pat. Nos. 8,404,631 and 8,404,632; WO 2007/139791, WO 2007/139767 and WO 2014/107171; and U.S. patent application Ser. No. 14/530,569, the contents of each is incorporated by reference in its entirety.

All references cited herein, including patent applications and publications, are incorporated herein by reference in their entirety.

### BRIEF SUMMARY OF THE INVENTION

The invention provides methods for modulating tumor immunity in an individual with a tumor comprising administering to the individual an effective amount of an O₂ carrier polypeptide. In some embodiments, the invention provides methods for enhancing an immune response to the tumor. In some embodiments, the invention provides methods for increasing lymphocyte infiltration to a tumor in an individual comprising administering to the individual an effective amount of an O₂ carrier polypeptide. In some embodiments, the increase in lymphocyte infiltration to the tumor comprises an increase in infiltration of one or more of CD4 cells, CD8 cells, or NK cells. In some embodiments, the increase in lymphocyte infiltration to the tumor is accompanied by inhibition of one or more of Treg cells, tumor associated macrophages or myeloid derived suppressor cells in the tumor. In some embodiments, the increase in lymphocyte infiltration to the tumor is accompanied by an increase in MHC1 expression on the tumor cells. In some embodiments, the modulating of tumor immunity comprises increasing antigen processing. In some embodiments, the modulating of tumor immunity comprises increasing the presentation capabilities of dendritic cells (DC).

In some embodiments, the invention provides methods for decreasing expression of hypoxia inducible factor 1α (HIF-1α) and/or hypoxia inducible factor 2a (HIF-2a) in a tumor in an individual comprising administering to the individual an effective amount of an O₂ carrier polypeptide. In some embodiments, the invention provides methods for decreasing expression of programmed death ligand-1 (PD-L1) in a tumor in an individual comprising administering to the individual an effective amount of an O₂ carrier polypeptide. In some embodiments, the invention provides methods for decreasing expression of A2A adenosine receptor (A2AR) in a tumor in an individual comprising administering to the individual an effective amount of an O₂ carrier polypeptide.

In some embodiments of the above embodiments, the tumor is a brain tumor, a glioblastoma, a bone tumor, a pancreatic tumor, a skin tumor, a tumor of the head or neck, a melanoma, a lung tumor, a uterine tumor, an ovarian tumor, a colorectal tumor, a liver tumor, a hepatocellular carcinoma, a stomach tumor, a testicular tumor, an endometrial tumor, a cervical tumor, a vaginal tumor, a Hodgkin's lymphoma, a non-Hodgkin's lymphoma, an esophageal tumor, an intestinal tumor, a thyroid tumor, an adrenal tumor, a bladder tumor, a kidney tumor, breast tumor, a multiple myeloma tumor, a sarcoma, or a squamous cell tumor.

In some aspects, the invention provides methods for treating cancer in an individual comprising administering to the individual an effective amount of an O₂ carrier polypeptide. In some embodiments, the cancer is brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma, anal cancer or squamous cell cancer.

In some embodiments of the above aspects and embodiments, the individual is a mammal. In further embodiments, the mammal is a human (e.g., a human patient). In other embodiments, the mammal is a pet, a laboratory research animal, or a farm animal. In some embodiments, the pet, research animal or farm animal is a dog, a cat, a horse, a monkey, a rabbit, a rat, a mouse, a guinea pig, a hamster, a pig, or a cow.

In some embodiments of the above aspects and embodiments, the O₂ carrier polypeptide is administered by intravenous, intra-arterial, intratumoral, intravesicular, inhalation, intraperitoneal, intrapulmonary, intramuscular, subcutaneous, intra-tracheal, transmucosal, intraocular, intrathecal, or transdermal administration. In some embodiments, administration of the O₂ carrier polypeptide is repeated. In some embodiments, administration of the O₂ carrier polypeptide is repeated daily or twice a day from about 4 weeks to about 8 weeks. In some embodiments, the O₂ carrier polypeptide is administered every four, every 8, every 12 or every 24 hours for a period of about one to about 10 days. In some embodiments, the O₂ carrier polypeptide is administered as a bolus. In other embodiments, the O₂ carrier polypeptide is administered by infusion. In some embodiments, the O₂ carrier polypeptide is infused in the individual for about 15 minutes, about 30 minutes, about 1 hour, about 2 hours, about 3 hours, about 6 hours, about 12 hours, about 24 hours or more than 24 hours.

In some embodiments, the invention provides methods to modulate tumor immunity or to treat cancer in an individual wherein an O₂ carrier polypeptide is administered in combination with radiation therapy. In some embodiments, the radiation therapy is administered to the individual 1, 2, 3, 4, 5, 6, 8, 10, 12, 14, 16, 18, 20 or 24 hours after the O₂ carrier polypeptide is administered. In some embodiments, the radiation is X-radiation. In some embodiments, the X-radiation is administered at about 0.5 gray to about 75 gray. In some embodiments, the administration of the O₂ carrier polypeptide and/or the administration of the radiation is repeated. In some embodiments, the administration is repeated more than about any of two, three, four times, five times, ten times, 15 times, 20 times, 25 times or 30 times. In some embodiments, the administration is repeated after one week, two weeks, three weeks, or four weeks.

In some embodiments, the invention provides methods to modulate tumor immunity or to treat cancer in an individual wherein an O₂ carrier polypeptide is administered in combination with chemotherapy or immunotherapy. In some embodiments, the chemotherapy comprises a cytotoxin. In some embodiments, the administration of the O₂ carrier polypeptide and/or the administration of the chemotherapy is repeated. In some embodiments, the immunotherapy is one or more of an anticancer vaccine, an adoptive immune cell therapy or an agent that targets an immune checkpoint regulator. In some embodiments, the immunotherapy targets one or more of CTLA-4, PD1, PD-L1, or an immune checkpoint regulator. In some embodiments, the adoptive immune therapy is a chimeric antigen receptor expressing T cell or an engineered TCR-T cell. In some embodiments, the immune therapy is an oncolytic virus or a Bispecific T cell Engager (BiTE). In some embodiments, the administration of the O₂ carrier polypeptide and/or the administration of the immunotherapy is repeated.

In some embodiments of the above embodiments, the O₂ carrier polypeptide is in a pharmaceutical composition. In some embodiments, the pharmaceutical composition further comprises a pharmaceutically acceptable carrier. In some embodiments of any of the above embodiments, the O₂ carrier polypeptide is an H-NOX protein.

In some aspects, the invention provides methods for modulating tumor immunity in an individual with a tumor comprising administering to the individual an effective amount of an H-NOX protein. In some embodiments, the invention provides methods for enhancing an immune response to the tumor. In some embodiments, the invention provides methods for increasing leucocyte infiltration to a tumor in an individual comprising administering to the individual an effective amount of an H-NOX protein. In some embodiments, the invention provides methods for increasing lymphocyte infiltration to a tumor in an individual comprising administering to the individual an effective amount of an H-NOX protein. In some embodiments, the increase in lymphocyte infiltration to the tumor comprises an increase in infiltration of one or more of CD4 cells, CD8 cells, or NK cells. In some embodiments, the increase in lymphocyte infiltration to the tumor is accompanied by inhibition of one or more of Treg cells, tumor associated macrophages or myeloid derived suppressor cells in the tumor. In some embodiments, the increase in lymphocyte infiltration to the tumor is accompanied by an increase in MHC1 expression on the tumor cells. In some embodiments, the modulating of tumor immunity comprises increasing antigen processing. In some embodiments, the modulating of tumor immunity comprises increasing lymphocyte activation. In some embodiments, the modulating of tumor immunity comprises increasing the presentation capabilities of dendritic cells (DC).

In some embodiments, the invention provides methods for decreasing expression of hypoxia inducible factor 1α (HIF-1α) and/or hypoxia inducible factor 2a (HIF-2α) in a tumor in an individual comprising administering to the individual an effective amount of an H-NOX protein. In some embodiments, the invention provides methods for decreasing expression of programmed death ligand-1 (PD-L1) in a tumor in an individual comprising administering to the individual an effective amount of an H-NOX protein. In some embodiments, the invention provides methods for decreasing expression of A2A adenosine receptor (A2AR) in a tumor in an individual comprising administering to the individual an effective amount of an H-NOX protein.

In some embodiments of the above embodiments, the tumor is a brain tumor, a glioblastoma, a bone tumor, a pancreatic tumor, a skin tumor, a tumor of the head or neck, a melanoma, a lung tumor, a uterine tumor, an ovarian tumor, a colorectal tumor, an anal tumor, a liver tumor, a hepatocellular carcinoma, a stomach tumor, a testicular tumor, an endometrial tumor, a cervical tumor, a vaginal tumor, a Hodgkin's lymphoma, a non-Hodgkin's lymphoma, an esophageal tumor, an intestinal tumor, a thyroid tumor, an adrenal tumor, a bladder tumor, a kidney tumor, breast tumor, a multiple myeloma tumor, a sarcoma, or a squamous cell tumor.

In some aspects, the invention provides methods for treating cancer in an individual comprising administering to the individual an effective amount of an H-NOX protein. In some embodiments, the cancer is brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma or squamous cell cancer.

In some embodiments of the above aspects and embodiments, the individual is a mammal. In further embodiments, the mammal is a human (e.g., a human patient). In other embodiments, the mammal is a pet, a laboratory research animal, or a farm animal. In some embodiments, the pet, research animal or farm animal is a dog, a cat, a horse, a monkey, a rabbit, a rat, a mouse, a guinea pig, a hamster, a pig, or a cow.

In some embodiments of the above aspects and embodiments, the H-NOX protein is administered by intravenous, intra-arterial, intratumoral, intravesicular, inhalation, intraperitoneal, intrapulmonary, intramuscular, subcutaneous, intra-tracheal, transmucosal, intraocular, intrathecal, or transdermal administration. In some embodiments, administration of the H-NOX protein is repeated. In some embodiments, administration of the H-NOX protein is repeated daily or twice a day from about 4 weeks to about 8 weeks. In some embodiments, the H-NOX protein is administered every four, every 8, every 12, every 24 hours, or every 48 hours for a period of about one to about 10 days. In some embodiments, the H-NOX protein is administered as a bolus. In other embodiments, the H-NOX protein is administered by infusion. In some embodiments, the H-NOX protein is infused in the individual for about 15 minutes, about 30 minutes, about 1 hour, about 2 hours, about 3 hours, about 6 hours, about 12 hours, about 24 hours or more than 24 hours.

In some embodiments, the invention provides methods to modulate tumor immunity or to treat cancer in an individual wherein an H-NOX protein is administered in combination with radiation therapy. In some embodiments, the radiation therapy is administered to the individual 1, 2, 3, 4, 5, 6, 8, 10, 12, 14, 16, 18, 20 or 24 hours after the H-NOX protein is administered. In some embodiments, the radiation is X-radiation. In some embodiments, the X-radiation is administered at about 0.5 gray to about 75 gray. In some embodiments, the administration of the H-NOX protein and/or the administration of the radiation is repeated. In some embodiments, the administration is repeated more than about any of two, three, four times, five times, ten times, 15 times, 20 times, 25 times, 30 times or 40 times. In some embodiments, the administration is repeated after one week, two weeks, three weeks, or four weeks or more.

In some embodiments, the invention provides methods to modulate tumor immunity or to treat cancer in an individual wherein an H-NOX protein is administered in combination with chemotherapy or immunotherapy. In some embodiments, the chemotherapy comprises a cytotoxin. In some embodiments, the administration of the H-NOX protein and/or the administration of the chemotherapy is repeated. In some embodiments, the immunotherapy is one or more of an anticancer vaccine, an adoptive immune cell therapy or an agent that targets an immune checkpoint regulator. In some embodiments, the immunotherapy targets one or more of CTLA-4, PD1, PD-L1, or an immune checkpoint regulator. In some embodiments, the adoptive immude therapy is a chimeric antigen receptor expressing T cell or an engineered TCR-T cell. In some embodiments, the immune therapy is an oncolytic virus or a Bispecific T cell Engager (BiTE). In some embodiments, the administration of the H-NOX protein and/or the administration of the immunotherapy is repeated.

In some embodiments of the above aspects and embodiments, the H-NOX protein is a T. tengcongensis H-NOX, a L. pneumophilia 2 H-NOX, a H. sapiens B1, a R. norvegicus β1, a C. lupus H-NOX, a D. melangaster B1, a D. melangaster CG14885-PA, a C. elegans GCY-35, a N. punctiforme H-NOX, C. crescentus H-NOX, a S. oneidensis H-NOX, or C. acetobutylicum H-NOX. In some embodiments, the H-NOX protein comprises a H-NOX domain corresponding to the H-NOX domain of T. tengcongensis set forth in SEQ ID NO:2.

In some embodiments, the H-NOX comprises one or more distal pocket mutations. In some embodiments, the distal pocket mutation is an amino acid substitution at a site corresponding to L144 of T. tengcongensis H-NOX. In some embodiments, the H-NOX is a T. tengcongensis H-NOX comprising an amino acid substitution at position 144. In some embodiments, the amino acid substitution at position 144 is an L144F substitution.

In some embodiments, the H-NOX protein is a polymeric H-NOX protein. In some embodiments, the polymeric H-NOX protein comprises monomers, wherein the monomers comprise an H-NOX domain and a polymerization domain. In some embodiments, the H-NOX domain is covalently linked to the polymerization domain. In some embodiments, the polymeric H-NOX protein is a trimeric H-NOX protein. In some embodiments, the trimeric H-NOX protein comprises one or more trimerization domains. In some embodiments, the trimeric H-NOX protein comprises three monomers, wherein the monomers comprise an H-NOX domain and a trimerization domain, wherein the trimerization domain is a bacteriophage T4 trimerization domain. In some embodiments, the trimerization domain is a foldon domain. In some embodiments, the foldon domain comprises the amino acid sequence of SEQ ID NO:4.

In some embodiments, the H-NOX protein is fused to an Fc domain of an immunoglobulin. In some embodiments, the H-NOX protein is covalently bound to polyethylene glycol.

In some embodiments, the O₂ dissociation constant of the H-NOX protein is within 2 orders of magnitude of that of hemoglobin, and wherein the NO reactivity of the H-NOX protein is at least 10-fold lower than that of hemoglobin. In some embodiments, the O₂ dissociation constant of the polymeric H-NOX protein is between about 1 nM and about 1000 nM at 20° C. In some embodiments, the O₂ dissociation constant of the H-NOX protein is between about 1 μM and about 10 μM at 20° C. In some embodiments, the O₂ dissociation constant of the H-NOX protein is between about 10 μM and about 50 μM at 20° C. In some embodiments, the NO reactivity of the H-NOX protein is less than about 700 s⁻¹ at 20° C. In some embodiments, the NO reactivity of the H-NOX protein is at least 100-fold lower than that of hemoglobin. In some embodiments, the NO reactivity of the H-NOX protein is at least 1,000-fold lower than that of hemoglobin. In some embodiments, the k_{off} for oxygen of the H-NOX protein is less than or equal to about 0.65 s⁻¹ at 20° C. In some embodiments, the k_{off} for oxygen of the H-NOX protein is between about 0.21 s-land about 0.65 s⁻¹ at 20° C. In some embodiments, the k_{off} for oxygen of the H-NOX protein is between about 1.35 s⁻¹ and about 2.9 s⁻¹ at 20° C. In some embodiments, the rate of heme autoxidation of the H-NOX protein is less than about 1 h⁻¹ at 37° C.

In some embodiments of the above embodiments, the H-NOX protein is in a pharmaceutical composition. In some embodiments, the pharmaceutical composition further comprises a pharmaceutically acceptable carrier.

In some aspects the invention provides the use of an O₂ carrier protein for modulating tumor immunity in an individual. In some embodiments, the modulating tumor immunity comprises enhancing an immune response to the tumor. In some embodiments, the invention provides the use of an O₂ carrier polypeptide for increasing leucocyte infiltration to a tumor in an individual. In some embodiments, the invention provides the use of an O₂ carrier polypeptide for increasing lymphocyte infiltration to a tumor in an individual. In some embodiments, the increase in lymphocyte infiltration to the tumor comprises an increase in infiltration of one or more of CD4 cells, CD8 cells, or NK cells. In some embodiments, the increase in lymphocyte infiltration to the tumor is accompanied by inhibition of one or more of Treg cells, tumor associated macrophages or myeloid derived suppressor cells in the tumor. In some embodiments, the increase in leucocyte infiltration to the tumor is accompanied by an increase in MHC1 expression on the tumor cells. In some embodiments, the increase in lymphocyte infiltration to the tumor is accompanied by an increase in MHC1 expression on the tumor cells.

In some embodiments, the invention provides the use of an O₂ carrier polypeptide for decreasing expression of HIF-1α and/or HIF-2α in a tumor in an individual. In some embodiments, the invention provides the use of an O₂ carrier polypeptide for decreasing expression of PD-L1 in a tumor in an individual. In some embodiments, the invention provides the use of an O₂ carrier polypeptide for decreasing expression of A2AR in a tumor in an individual.

In some embodiments of the above uses, the tumor is a brain tumor, a glioblastoma, a bone tumor, a pancreatic tumor, a skin tumor, a tumor of the head or neck, a melanoma, a lung tumor, a uterine tumor, an ovarian tumor, a colorectal tumor, an anal tumor, a liver tumor, a hepatocellular carcinoma, a stomach tumor, a testicular tumor, an endometrial tumor, a cervical tumor, a vaginal tumor, a Hodgkin's lymphoma, a non-Hodgkin's lymphoma, an esophageal tumor, an intestinal tumor, a thyroid tumor, an adrenal tumor, a bladder tumor, a kidney tumor, a breast tumor, a multiple myeloma tumor, a sarcoma, or a squamous cell tumor.

In some embodiments, the invention provides the use of an O₂ carrier protein for treating cancer in an individual. In some embodiments, the cancer is brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma, or squamous cell cancer.

In some embodiments of the above uses, the individual is a mammal. In some embodiments, the mammal is a human.

In some embodiments of the above uses, the O₂ carrier polypeptide is an H-NOX protein. In some embodiments, the H-NOX protein is a T. tengcongensis H-NOX, a L . . . pneumophilia 2 H-NOX, a H. sapiens B1, a R. norvegicus B1, a C. lupus H-NOX, a D. melangaster B1, a D. melangaster CG14885-PA, a C. elegans GCY-35, a N. punctiforme H-NOX, C. crescentus H-NOX, a S. oneidensis H-NOX, or C. acetobutylicum H-NOX. In some embodiments, the H-NOX protein comprises a H-NOX domain corresponding to the H-NOX domain of T. tengcongensis set forth in SEQ ID NO:2. In some embodiments, the H-NOX comprises one or more distal pocket mutations. In some embodiments, the distal pocket mutation is an amino acid substitution at a site corresponding to L144 of T. tengcongensis H-NOX. In some embodiments, the H-NOX is a T. tengcongensis H-NOX comprising an amino acid substitution at position 144. In some embodiments, the amino acid substitution at position 144 is an L144F substitution.

In some embodiments, the H-NOX protein is a polymeric H-NOX protein. In some embodiments, the polymeric H-NOX protein comprises monomers, wherein the monomers comprise an H-NOX domain and a polymerization domain. In some embodiments, the H-NOX domain is covalently linked to the polymerization domain. In some embodiments, the polymeric H-NOX protein is a trimeric H-NOX protein. In some embodiments, the trimeric H-NOX protein comprises one or more trimerization domains. In some embodiments, the trimeric H-NOX protein comprises three monomers, wherein the monomers comprise an H-NOX domain and a trimerization domain, wherein the trimerization domain is a bacteriophage T4 trimerization domain. In some embodiments, the trimerization domain is a foldon domain. In some embodiments, the foldon domain comprises the amino acid sequence of SEQ ID NO:4.

In some embodiments, the H-NOX protein is fused to an Fc domain of an immunoglobulin. In some embodiments, the H-NOX protein is covalently bound to polyethylene glycol.

In some aspects, the invention provides kits for modulating tumor immunity in an individual comprising an O₂ carrier protein for use in the methods described herein. In some embodiments, the kit further comprises one or more of a vial, a vessel, an ampule, a bottle, a jars, or flexible packaging. In some embodiments, the kit further comprises one or more buffer. In some embodiments, the kit further comprises instructions for use.

### BRIEF DESCRIPTION OF THE DRAWINGS

FIGS. 1A and 1B shows a model of the major immunosuppressive pathways promoted by hypoxia (FIG. 1A) and the points of therapeutic intervention that may be exerted by O₂ carrier polypeptide treatment (FIG. 1B).

FIGS. 2A-2C show tumor oxygenation after single bolus dose of PEGylated trimer Tt H-NOX L144F assessed by pimonidazole and HIF-1 ELISA. FIG. 2A shows pimonidazole levels measured by competitive ELISA. FIG. 2B shows HIF-1α levels measured by sandwich ELISA. Graphs show quantification of pimonidazole and HIF-1α signals after PEGylated trimer Tt H-NOX L144F administration. Mean values+/−SEM. ***p<0.001, **p<0.01 by One way ANOVA and Bonferroni's post-hoc tests. FIG. 2C shows assessment of tumors for the accumulation of PEGylated trimer Tt H-NOX L144F by sandwich H-NOX ELISA and results expressed per gram of tumor tissue.

FIGS. 3A-3D show direct measurements of tumor tissue oxygenation following PEGylated trimer Tt H-NOX L144F administration. Tumors were treated either with PEGylated trimer Tt H-NOX L144F (FIG. 3A), non-functional control Tt H-NOX protein (FIG. 3C), or with 100% oxygen starting at pO₂=0.44 mmHg (FIG. 3B) with 100% oxygen starting at pO₂=5 mmHg (FIG. 3D).

FIG. 4 shows enhancement of radiation efficacy following PEGylated trimer Tt H-NOX L144F treatment of mice bearing H460 tumors. Mice bearing H460 subcutaneous xenograft tumors (150-300 mm³) were either pre-treated with PEGylated trimer Tt H-NOX L144F or treated with 10 Gy alone, irradiated, tumors extracted and processed for clonogenic assay. Cell numbers were counted 7 days later in triplicate samples from each tumor. Each dot on the graph represents average surviving fraction for one tumor.

FIGS. 5A-5C show PEGylated trimer Tt H-NOX L144F downregulates HIF-1α targets involved in immunosuppression. Mice bearing H460 subcutaneous xenograft tumors (150-300 mm³) were either pre-treated with PEGylated trimer Tt H-NOX L144 For treated with vehicle alone, and harvested for qRT-PCR analysis. FIG. 5A shows expression of VEGF. FIG. 5B shows expression of GLUT1. FIG. 5C shows expression of PD-L1.

FIG. 6A shows the nucleic acid (SEQ ID NO:5) and amino acid sequence (SEQ ID NO: 6) of the foldon domain of bacteriophage T4 fibritin fused to the C-terminus of a Thermoanaerobacter tengcongensis L144F H-NOX sequence and including the His6 tag. FIG. 6B shows the nucleic acid (SEQ ID NO:7) and amino acid sequence (SEQ ID NO:8) of the L144F H-NOX-foldon monomer without a His6 tag.

FIGS. 7A-7C show representative images of tumor hypoxia and T cell infiltration is B16F10 subcutaneous tumors (FIG. 7A), CT26 subcutaneous tumors (FIG. 7B) and GL261 intracranial tumors (FIG. 7C). Hypoxia (top panels) and T cell infiltration (middle panels) is shown by immunohistochemistry. Bottom panels show results of quantitative analysis of multiple tumor sections. Significantly fewer CD4 and CD8 T cells infiltrate the hypoxic regions of tumors.

FIG. 8 shows quantification of CD8 T cells in hypoxic areas of tumors after H-NOX treatment (OMX) or vehicle control treatment (Veh). Representative images are shown. Hypoxic areas were labeled with pimondazole by immunohistochemical analysis. Following OMX treatment there is an increase in CD4 (data not shown) and CD8 T cell infiltration into regions of tumors that were hypoxic prior to OMX administration.

FIGS. 9A and 9B show quantification of T cells in normoxic and hypoxic areas of tumors after H-NOX treatment (OMX) or vehicle control treatment (Veh). Both CD4 and CD8 T cells were evaluated. Tumor areas evaluated included areas on the periphery of the tumor and in the tumor center. Results of quantitative image analysis of multiple sections are shown in FIG. 9A and representative images in FIG. 9B. Hypoxic areas were labelled using immunohistochemical analysis of carbonic anhydrase IX (CAIX) expression. Following OMX treatment, there is an increase in CD4 and CD8 T cell infiltration into regions of tumors that were hypoxic prior to OMX administration.

FIG. 10 shows the results of immunohistochemistry for hypoxia (pimondazole) and CD3 vessels is GL261 tumor model.

FIG. 11 shows immunohistochemical analysis of H-NOX tumor penetration, tumor hypoxia and CD8 T cell localization in canine oral melanoma tumors. Tissues were stained with hematoxylin and eosin (H&E), DNA interchelating dye (DAPI) and with anti-H-NOX (OMX), -carbonic anhydrase IX (CAIX) and −CD8 antibodies to assess CD8 lymphocyte localization in tumor regions that were hypoxic prior to H-NOX (OMX) treatment. Images reveal CD8 positive T cells localized throughout regions of the tumor that were hypoxic prior to H-NOX (OMX) treatment (CAIX positive).

FIGS. 12A-12K show that larger tumor size correlates with enhanced hypoxia and reduced lymphocyte infiltration in subcutaneous 4T1-Luc syngeneic mouse tumors. FIG. 12A shows tumor volumes on day 10 and day 14 post-implant. FIG. 12B shows fraction of lymphocytes within the viable cell population. FIG. 12C shows the absolute lymphocyte cell numbers within the viable population. FIG. 12D shows a negative correlation between tumor volume and percentage lymphocytes. FIG. 12E shows a positive correlation between tumor volume and percentage hypoxia. FIG. 12F shows a negative correlation between percentage hypoxia and percentage lymphocytes. FIG. 12G shows a negative correlation between tumor volume and percentage CD3-positive T cells. FIG. 12H shows a negative correlation between tumor volume and percentage CD4-positive T cells. FIG. 12I shows a negative correlation between tumor volume and percentage CD8-positive T cells. FIG. 12J shows a negative correlation between tumor volume and percentage CD3-CD4-double-positive T cells. FIG. 12K shows a negative correlation between tumor volume and percentage CD3-CD8-double-positive T cells.

FIGS. 13A-13F shows that hypoxic tumor regions are immunosuppressive and exhibit reduced T cell infiltration in subcutaneous 4T1-Luc syngeneic mouse tumors. Immunofluorescence staining of tumor region #1 for (FIG. 13A) pimonidazole-positive hypoxic areas and (FIG. 13B) CD8-positive T cells, counterstained with (FIG. 13C) DAPI to highlight nuclei. Immunofluorescence staining of tumor region #2 for (FIG. 13D) pimonidazole-positive hypoxic areas and (FIG. 13E) CD4-positive T cells, counterstained with (FIG. 13F) DAPI to highlight nuclei.

### DETAILED DESCRIPTION OF THE INVENTION

The present invention provides methods for treating cancer in an individual comprising administering to the individual an effective amount of an O₂ carrier polypeptide such as an H-NOX protein. In certain aspects, the invention provides methods for modulating hypoxia-mediated tumor immunity in an individual comprising administering to the individual an effective amount of an O₂ carrier polypeptide, such as an H-NOX protein. The O₂ carrier polypeptide is delivered to the tumor where it enhances an immune response to the tumor. Enhancement of an immune response to the tumor may be mediated by targeting hypoxia inducible factor 1α (HIF-1α)-mediated pathways of tumor immunity and/or non-HIF-1α-mediated pathways of tumor immunity. In some aspects, the invention provides methods for increasing lymphocyte infiltration to a tumor in an individual comprising administering to the individual an effective amount of an O₂ carrier polypeptide. In some embodiments, the increase in lymphocyte infiltration to the tumor comprises an increase in infiltration of one or more of CD4 cells, CD8 cells, or NK cells. In some embodiments, the increase in lymphocyte infiltration to the tumor is accompanied by inhibition of one or more of Treg cells, tumor associated macrophages or myeloid derived suppressor cells in the tumor. In some embodiments, the increase in lymphocyte infiltration to the tumor is accompanied by an increase in MHC1 expression on the tumor cells. In some embodiments, the invention provides methods for decreasing expression of hypoxia inducible factor 1α (HIF-1α) in a tumor in an individual comprising administering to the individual an effective amount of an O₂ carrier polypeptide (e.g., an H-NOX protein). In some embodiments, the invention provides methods for decreasing expression of programmed death ligand-1 (PD-L1) in a tumor in an individual comprising administering to the individual an effective amount of an O₂ carrier polypeptide (e.g., an H-NOX protein). In some embodiments, the invention provides methods for decreasing expression of A2A adenosine receptor (A2AR) in a tumor in an individual comprising administering to the individual an effective amount of an O₂ carrier polypeptide (e.g., an H-NOX protein).

### Definitions

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention.

For use herein, unless clearly indicated otherwise, use of the terms “a”, “an,” and the like refers to one or more.

In this application, the use of “or” means “and/or” unless expressly stated or understood by one skilled in the art. In the context of a multiple dependent claim, the use of “or” refers back to more than one preceding independent or dependent claim.

Reference to “about” a value or parameter herein includes (and describes) embodiments that are directed to that value or parameter per se. For example, description referring to “about X” includes description of “X.”

It is understood that aspect and embodiments of the invention described herein include “comprising,” “consisting,” and “consisting essentially of” aspects and embodiments.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and polymers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. As used herein, a protein may include two or more subunits, covalently or non-covalently associated; for example, a protein may include two or more associated monomers.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

As used herein, the term “hypoxia inducible factor” or “HIF” refers to a family of transcription factor that respond to decreases in oxygen, or hypoxia, in the cellular environment. Members of the human HIF family include HIF-1a, HIF-1B, HIF-2a, HIF-2B, HIF3α, HIF3β. HIF-1 functions as a master regulator of homeostatic responses to hypoxia by activating transcription of many genes, including those involved in energy metabolism, angiogenesis, apoptosis, and other genes whose protein products increase oxygen delivery or facilitate metabolic adaptation to hypoxia. HIF-1 plays a role in embryonic vascularization, tumor angiogenesis and pathophysiology of ischemic disease. Human hypoxia-inducible factor 1, alpha subunit or human HIF-1α interacts with a number of polypeptides including but not limited to ARNTL, ARNT, CREBB, EP300, HIF-1AN, Mdm2, NR4A, p53, PSMA7, STAT3, UBC, VH and pVHL. Human HIF-1α is encoded by the HIF-1A gene. The amino acid sequence of human HIF-1α is provided by GenBank Accession no. NP_001230013 and the nucleotide sequence of human HIF-1α mRNA is provided by GenBank Accession No. NM_001243084. The amino acid sequence of mouse HIF-1α is provided by GenBank Accession no. NP_010431 and the nucleotide sequence of mouse HIF-1α mRNA is provided by GenBank Accession No. NM_034561.

As used herein, “programmed death-ligand 1” or “PD-L1” refers to a transmembrane protein that is part of an immune checkpoint pathway that plays a role in suppressing the immune system. Interaction of PDL1 with the PD1 receptor or the B7.1 receptor inhibits T cell receptor-mediated activation of IL-2 and T cell proliferation. Human PD-L1 is encoded by the CD274 gene. The amino acid sequence of human PD-L1 is provided by GenBank Accession no. NP_001254635 and the nucleotide sequence of human PD-L1 mRNA is provided by GenBank Accession No. NM_001267706. The amino acid sequence of mouse PD-L1 is provided by GenBank Accession no. NP_021893 and the nucleotide sequence of mouse PD-L1 mRNA is provided by GenBank Accession No. NM_068693.

As used herein, an “adenosine A2A receptor” or “A2AR” refers to a receptor of the G protein-coupled receptor superfamily. A2AR is a receptor for adenosine that plays a role in oxygen consumption and is thought to play a role in suppressing overreactive immune cells by way or increased levels of CAMP. Human A2AR is encoded by ADORA2A the gene. The amino acid sequence of human A2AR is provided by GenBank Accession no. NP_000666 and the nucleotide sequence of human A2AR mRNA is provided by GenBank Accession No. NM_000675. The amino acid sequence of mouse A2AR is provided by GenBank Accession no. NP 033760 and the nucleotide sequence of mouse A2AR mRNA is provided by GenBank Accession No. NM_09630.

As used herein, an “H-NOX protein” means a protein that has an H-NOX domain (named for Heme-Nitric oxide and OXygen binding domain). An H-NOX protein may or may not contain one or more other domains in addition to the H-NOX domain. In some examples, an H-NOX protein does not comprise a guanylyl cyclase domain. An H-NOX protein may or may not comprise a polymerization domain.

As used herein, a “polymeric H-NOX protein” is an H-NOX protein comprising two or more H-NOX domains. The H-NOX domains may be covalently or non-covalently associated.

As used herein, an “H-NOX domain” is all or a portion of a protein that binds nitric oxide and/or oxygen by way of heme. The H-NOX domain may comprise heme or may be found as an apoproprotein that is capable of binding heme. In some examples, an H-NOX domain includes six alpha-helices, followed by two beta-strands, followed by one alpha-helix, followed by two beta strands. In some examples, an H-NOX domain corresponds to the H-NOX domain of Thermoanaerobacter tengcongensis H-NOX set forth in SEQ ID NO:2. For example, the H-NOX domain may be at least about 10%, 15%, 20%, 25%, 30%, 40%, 50%, 60%, 70%, 80%, 90%, 95%, or 99% identical to the H-NOX domain of Thermoanaerobacter tengcongensis H-NOX set forth in SEQ ID NO:2. In some embodiments, the H-NOX domain may be 10%-20%, 20%-30%, 30%-40%, 40%-50%, 50%-60%, 60%-70%, 70%-80%, 80%-90%, 90%-95%, 95%-99% or 100% identical to the H-NOX domain of Thermoanaerobacter tengcongensis H-NOX set forth in SEQ ID NO:2.

As used herein, a “polymerization domain” is a domain (e.g. a polypeptide domain) that promotes the association of monomeric moieties to form a polymeric structure. For example, a polymerization domain may promote the association of monomeric H-NOX domains to generate a polymeric H-NOX protein. An exemplary polymerization domain is the foldon domain of T4 bacteriophage, which promotes the formation of trimeric polypeptides. Other examples of polymerization domains include, but are not limited to, Arc, POZ, coiled coil domains (including GCN4, leucine zippers, Velcro), uteroglobin, collagen, 3-stranded coiled colis (matrilin-1), thrombosporins, TRPV1-C, P53, Mnt, avadin, streptavidin, Bcr-Abl, COMP, verotoxin subunit B, CamKII, RCK, and domains from N ethylmaleimide-sensitive fusion protein, STM3548, KaiC, TyrR, Hcp1, CcmK4, GP41, anthrax protective antigen, aerolysin, a-hemolysin, C4b-binding protein, Mi-CK, arylsurfatase A, and viral capsid proteins.

As used herein, an “amino acid linker sequence” or an “amino acid spacer sequence” is a short polypeptide sequence that may be used to link two domains of a protein. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence.

As used herein, a “His₆ tag” refers to a peptide comprising six His residues attached to a polypeptide. A His₆ tag may be used to facilitate protein purification; for example, using chromatography specific for the His₆ tag. Following purification, the His₆ tag may be cleaved using an exopeptidase.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially reduced,” or “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, or 90%. In some embodiment, the two substantially different numeric values differ by about any one of 10%-20%, 20%-30%, 30%-40%, 40%-50%, 50%-60%, 60%-70%, 70%-80%, 80%-90%, 90%-95%, 95%-99% or 100%.

A “native sequence” polypeptide comprises a polypeptide having the same amino acid sequence as a polypeptide found in nature. Thus, a native sequence polypeptide can have the amino acid sequence of naturally occurring polypeptide from any organism. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about any one of 80%, 90% or 95% amino acid sequence identity with the native sequence polypeptide. In some embodiments, a variant will have about any one of 80%-90%, 90%-95% or 95%-99% amino acid sequence identity with the native sequence polypeptide.

As used herein, a “mutant protein” means a protein with one or more mutations compared to a protein occurring in nature. In one embodiment, the mutant protein has a sequence that differs from that of all proteins occurring in nature. In various embodiments, the amino acid sequence of the mutant protein is at least about any of 10, 15, 20, 25, 30, 40, 50, 60, 70, 80, 90, 95, 97, 98, 99, or 99.5% identical to that of the corresponding region of a protein occurring in nature. In some embodiments, the amino acid sequence of the mutant protein is at least about any of 10%-20%, 20%-30%, 30%-40%, 40%-50%, 50%-60%, 60%-70%, 70%-80%, 80%-90%, 90%-95%, 95%-99% or 100% identical to that of the corresponding region of a protein occurring in nature. In some embodiments, the mutant protein is a protein fragment that contains at least about any of 25, 50, 75, 100, 150, 200, 300, or 400 contiguous amino acids from a full-length protein. In some embodiments, the mutant protein is a protein fragment that contains about any of 25-50, 50-75, 75-100, 100-150, 150-200, 200-300, or 300-400 contiguous amino acids from a full-length protein. Sequence identity can be measured, for example, using sequence analysis software with the default parameters specified therein (e.g., Sequence Analysis Software Package of the Genetics Computer Group, University of Wisconsin Biotechnology Center, 1710 University Avenue, Madison, WI 53705). This software program matches similar sequences by assigning degrees of homology to various amino acids replacements, deletions, and other modifications.

As used herein, a “mutation” means an alteration in a reference nucleic acid or amino acid sequence occurring in nature. Exemplary nucleic acid mutations include an insertion, deletion, frameshift mutation, silent mutation, nonsense mutation, or missense mutation. In some embodiments, the nucleic acid mutation is not a silent mutation. Exemplary protein mutations include the insertion of one or more amino acids (e.g., the insertion of 2, 3, 4, 5, 6, 7, 8, 9, or 10 amino acids), the deletion of one or more amino acids (e.g., a deletion of N-terminal, C-terminal, and/or internal residues, such as the deletion of at least about any of 5, 10, 15, 25, 50, 75, 100, 150, 200, 300, or more amino acids or a deletion of about any of 5-10, 10-15, 15-25, 25-50, 50-75, 75-100, 100-150, 150-200, 200-300, or 300-400 amino acids), the replacement of one or more amino acids (e.g., the replacement of 1, 2, 3, 4, 5, 6, 7, 8, 9, or 10 amino acids), or combinations of two or more of the foregoing. The nomenclature used in referring to a particular amino acid mutation first identifies the wild-type amino acid, followed by the residue number and finally the substitute amino acid. For example, Y140L means that tyrosine has been replaced by a leucine at residue number 140. Likewise, a variant H-NOX protein may be referred to by the amino acid variations of the H-NOX protein. For example, a T. tengcongensis Y140L H-NOX protein refers to a T. tengcongensis H-NOX protein in which the tyrosine residue at position number 140 has been replaced by a leucine residue and a T. tengcongensis W9F/Y140L H-NOX protein refers to a T. tengcongensis H-NOX protein in which the tryptophan residue at position 9 has been replaced by a phenylalanine residue and the tyrosine residue at position number 140 has been replaced by a leucine residue.

An “evolutionary conserved mutation” is the replacement of an amino acid in one protein by an amino acid in the corresponding position of another protein in the same protein family.

As used herein, “derived from” refers to the source of the protein into which one or more mutations is introduced. For example, a protein that is “derived from a mammalian protein” refers to protein of interest that results from introducing one or more mutations into the sequence of a wild-type (i.e., a sequence occurring in nature) mammalian protein.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, a “k_{off}” refers to a dissociation rate, such as the rate of release of O₂ or NO from a protein. A lower numerical lower k_{off} indicates a slower rate of dissociation.

As used herein, “kₒₙ” refers to an association rate, such as the rate of binding of O₂ or NO to a protein. A lower numerical lower kₒₙ indicates a slower rate of association.

As used herein, “dissociation constant” refers to a “kinetic dissociation constant” or a “calculated dissociation constant.” A “kinetic dissociation constant” or “K_{D}” is a ratio of kinetic off-rate (k_{off}) to kinetic on-rate (kₒₙ), such as a K_{D} value determined as an absolute value using standard methods (e.g., standard spectroscopic, stopped-flow, or flash-photolysis methods) including methods known to the skilled artisan and/or described herein. “Calculated dissociation constant” or “calculated K_{D}” refers to an approximation of the kinetic dissociation constant based on a measured k_{off}. A value for the kₒₙ is derived via the correlation between kinetic K_{D} and k_{off} as described herein.

As used herein, “oxygen affinity” is a qualitative term that refers to the strength of oxygen binding to the heme moiety of a protein. This affinity is affected by both the k_{off} and kₒₙ for oxygen. A numerically lower oxygen K_{D} value means a higher affinity.

As used herein, “NO affinity” is a qualitative term that refers to the strength of NO binding to a protein (such as binding to a heme group or to an oxygen bound to a heme group associated with a protein). This affinity is affected by both the k_{off} and kₒₙ for NO. A numerically lower NO K_{D} value means a higher affinity.

As used herein, “NO stability” refers to the stability or resistance of a protein to oxidation by NO in the presence of oxygen. For example, the ability of the protein to not be oxidized when bound to NO in the presence of oxygen is indicative of the protein's NO stability. In some embodiments, less than about any of 50, 40, 30, 10, or 5% of an H-NOX protein is oxidized after incubation for about any of 1, 2, 4, 6, 8, 10, 15, or 20 hours at 20° C.

As used herein, “NO reactivity” refers to the rate at which iron in the heme of a heme-binding protein is oxidized by NO in the presence of oxygen. A lower numerical value for NO reactivity in units of s⁻¹ indicates a lower NO reactivity

As used herein, an “autoxidation rate” refers to the rate at which iron in the heme of a heme-binding protein is autoxidized. A lower numerical autoxidation rate in units of s⁻¹ indicates a lower autoxidation rate.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Exemplary prokaryotic cells include bacterial cells; for example, E. coli cells.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder.

A “disease” or “disorder” as used herein refers to a condition where treatment is needed.

The term “cancer” refers to a malignant proliferative disorder associated with uncontrolled cell proliferation, unrestrained cell growth, and decreased cell death via apoptosis.

The term “tumor” is used herein to refer to a group of cells that exhibit abnormally high levels of proliferation and growth. A tumor may be benign, pre-malignant, or malignant; malignant tumor cells are cancerous. Tumor cells may be solid tumor cells or leukemic tumor cells. The term “tumor growth” is used herein to refer to proliferation or growth by a cell or cells that comprise a tumor that leads to a corresponding increase in the size of the tumor.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment. Also encompassed by “treatment” is a reduction of pathological consequence of a proliferative disease. The methods of the invention contemplate any one or more of these aspects of treatment.

In the context of cancer, the term “treating” includes any or all of: inhibiting growth of tumor cells or cancer cells, inhibiting replication of tumor cells or cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or 99%.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

“Preventing,” as used herein, includes providing prophylaxis with respect to the occurrence or recurrence of a disease in a subject that may be predisposed to the disease but has not yet been diagnosed with the disease.

An “effective amount” of an agent refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile and essentially free of endotoxins.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

### H-NOX Proteins

### Overview of H-NOX Protein Family

Unless otherwise indicated, any wild-type or mutant H-NOX protein can be used in the compositions, kits, and methods as described herein. As used herein, an “H-NOX protein” means a protein that has an H-NOX domain (named for Heme-Nitric oxide and Oxygen binding domain). An H-NOX protein may or may not contain one or more other domains in addition to the H-NOX domain. H-NOX proteins are members of a highly-conserved, well-characterized family of hemoproteins (Iyer, L. M. et al. (Feb. 3, 2003). BMC Genomics 4 (1): 5; Karow, D. S. et al. (Aug. 10, 2004). Biochemistry 43 (31): 10203-10211; Boon, E. M. et al. (2005). Nature Chem. Biol. 1:53-59; Boon, E. M. et al. (October 2005). Curr. Opin. Chem. Biol. 9 (5): 441-446; Boon, E. M. et al. (2005). J. Inorg. Biochem. 99 (4): 892-902). H-NOX proteins are also referred to as Pfam 07700 proteins or HNOB proteins (Pfam-A database of protein domain family alignments and Hidden Markov Models, Copyright (C) 1996-2006 The Pfam Consortium; GNU LGPL Free Software Foundation, Inc., 59 Temple Place-Suite 330, Boston, MA 02111-1307, USA). In some embodiments, an H-NOX protein has, or is predicted to have, a secondary structure that includes six alpha-helices, followed by two beta-strands, followed by one alpha-helix, followed by two beta-strands. An H-NOX protein can be an apoprotein that is capable of binding heme or a holoprotein with heme bound. An H-NOX protein can covalently or non-covalently bind a heme group. Some H-NOX proteins bind NO but not O₂, and others bind both NO and O₂. H-NOX domains from facultative aerobes that have been isolated bind NO but not O₂. H-NOX proteins from obligate aerobic prokaryotes, C. elegans, and D. melanogaster bind NO and O₂. Mammals have two H-NOX proteins: β1 and β2. An alignment of mouse, rat, cow, and human H-NOX sequences shows that these species share >99% identity. In some embodiments, the H-NOX domain of an H-NOX protein or the entire H-NOX protein is at least about any of 10, 15, 20, 25, 30, 40, 50, 60, 70, 80, 90, 95, 97, 98, 99, or 99.5% identical to that of the corresponding region of a naturally-occurring Thermoanaerobacter tengcongensis H-NOX protein (e.g. SEQ ID NO:2) or a naturally-occurring sGC protein (e.g., a naturally-occurring sGC β1 protein). In some embodiments, the H-NOX domain of an H-NOX protein or the entire H-NOX protein is at least about any of 10-20%, 20-30%, 30-40%, 40-50%, 50-60%, 60-70%, 70-80%, 80-90%, 90-95%, 95-99, or 99-99.9% identical to that of the corresponding region of a naturally-occurring Thermoanaerobacter tengcongensis H-NOX protein (e.g. SEQ ID NO: 2) or a naturally-occurring sGC protein (e.g., a naturally-occurring sGC β1 protein). As discussed further herein, an H-NOX protein may optionally contain one or more mutations relative to the corresponding naturally-occurring H-NOX protein. In some embodiments, the H-NOX protein includes one or more domains in addition to the H-NOX domain. In particular embodiments, the H-NOX protein includes one or more domains or the entire sequence from another protein. For example, the H-NOX protein may be a fusion protein that includes an H-NOX domain and part or all of another protein, such as albumin (e.g., human serum albumin). In some embodiments, only the H-NOX domain is present. In some embodiments, the H-NOX protein does not comprise a guanylyl cyclase domain. In some embodiments, the H-NOX protein comprises a tag; for example, a His₆ tag.

### Polymeric H-NOX Proteins

In some aspects, the invention provides polymeric H-NOX proteins comprising two or more H-NOX domains. The two or more H-NOX domains may be covalently linked or noncovalently linked. In some embodiments, the polymeric H-NOX protein is in the form of a dimer, a trimer, a tetramer, a pentamer, a hexamer, a heptamer, an octomer, a nanomer, or a decamer. In some embodiments, the polymeric H-NOX protein comprises homologous H-NOX domains. In some embodiments, the polymeric H-NOX protein comprises heterologous H-NOX domains; for example, the H-NOX domains may comprises amino acid variants of a particular species of H-NOX domain or may comprise H-NOX domains from different species. In some embodiments, at least one of the H-NOX domains of a polymeric H-NOX protein comprises a mutation corresponding to an L144F mutation of T. tengcongensis H-NOX. In some embodiments, at least one of the H-NOX domains of a polymeric H-NOX protein comprises a mutation corresponding to a W9F/L144F mutation of T. tengcongensis H-NOX. In some embodiments, the polymeric H-NOX proteins comprise one or more polymerization domains. In some embodiments, the polymeric H-NOX protein is a trimeric H-NOX protein. In some embodiments, the polymeric H-NOX protein comprises at least one trimerization domain. In some embodiments, the trimeric H-NOX protein comprises three T. tengcongensis H-NOX domains. In some embodiments the trimeric H-NOX domain comprises three T. tengcongensis L144F H-NOX domains (trimeric Tt H-NOX L144F). In some embodiments the trimeric H-NOX domain comprises three T. tengcongensis W9F/L144F H-NOX domains

In some aspects of the invention, the polymeric H-NOX protein comprises two or more associated monomers. The monomers may be covalently linked or noncovalently linked. In some embodiments, monomeric subunits of a polymeric H-NOX protein are produced where the monomeric subunits associate in vitro or in vivo to form the polymeric H-NOX protein. In some embodiments, the monomers comprise an H-NOX domain and a polymerization domain. In some embodiments, the polymerization domain is covalently linked to the H-NOX domain; for example, the C-terminus of the H-NOX domain is covalently linked to the N-terminus or the C-terminus of the polymerization domain. In other embodiments, the N-terminus of the H-NOX domain is covalently linked to the N-terminus or the C-terminus of the polymerization domain. In some embodiments, an amino acid spacer is covalently linked between the H-NOX domain and the polymerization domain. An “amino acid spacer” and an “amino acid linker” are used interchangeably herein. In some embodiments, at least one of the monomeric subunits of a polymeric H-NOX protein comprises a mutation corresponding to an L144F mutation of T. tengcongensis H-NOX. In some embodiments, at least one of the monomeric subunits of a polymeric H-NOX protein comprises a mutation corresponding to a W9F/L144F mutation of T. tengcongensis H-NOX. In some embodiments the polymeric H-NOX protein is a trimeric H-NOX protein. In some embodiments, the monomer of a trimeric H-NOX protein comprises an H-NOX domain and a foldon domain of T4 bacteriophage. In some embodiments, the monomer of a trimeric H-NOX protein comprises a T. tengcongensis H-NOX domain and a foldon domain. In some embodiments, the monomer of a trimeric H-NOX protein comprises a T. tengcongensis L144F H-NOX domain and a foldon domain. In some embodiments, the monomer of a trimeric H-NOX protein comprises a T. tengcongensis W9F/L144F H-NOX domain and a foldon domain. In some embodiments, the trimer H-NOX protein comprises three monomers, each monomer comprising a T. tengcongensis L144F H-NOX domain and a foldon domain. In some embodiments, the H-NOX domain is linked to the foldon domain with an amino acid linker; for example a Gly-Ser-Gly linker. In some embodiments, at least one H-NOX domain comprises a tag. In some embodiments, at least one H-NOX domain comprises a His₆ tag. In some embodiments, the His₆ tag is linked to the foldon domain with an amino acid linker; for example an Arg-Gly-Ser linker. In some embodiments, all of the H-NOX domains comprise a His₆ tag. In some embodiments, the trimeric H-NOX protein comprises the amino acid sequence set forth in SEQ ID NO:6 or SEQ ID NO:8.

The exemplary H-NOX domain from T. tengcongensis is approximately 26.7 kDal. In some embodiments, the polymeric H-NOX protein has an atomic mass greater than any of about 50 kDal, 75 kDal, 100 kDal, 125 kDal, to about 150 kDal.

The invention provides polymeric H-NOX proteins that show greater accumulation in one or more tissues in an individual compared to a corresponding monomeric H-NOX protein comprising a single H-NOX domain following administration of the H-NOX protein to the individual. A corresponding H-NOX protein refers to a monomeric form of the H-NOX protein comprising at least one of the H-NOX domains of the polymeric H-NOX protein. Tissues of preferential polymeric H-NOX accumulation include, but are not limited to tumors and tissue with damaged vasculature. In some embodiments the polymeric H-NOX protein persists in a mammal for at least about 1, 2, 3, 4, 6, 12 or 24 hours following administration of the H-NOX protein to the individual. In some embodiments the polymeric H-NOX protein persists in a mammal for about 1-2, 2-3, 3-4, 4-6, 6-12 or 12-24 hours following administration of the H-NOX protein to the individual In some embodiments, less than about 10% of the polymeric H-NOX is cleared from mammal by the kidneys within less than any of about 1 hour, 2 hours or 3 hours following administration of the H-NOX protein to the individual.

### Sources of H-NOX Proteins and H-NOX Domains

H-NOX proteins and H-NOX domains from any genus or species can be used in the compositions, kits, and methods described herein. In various embodiments, the H-NOX protein or the H-NOX domains of a polymeric H-NOX protein is a protein or domain from a mammal (e.g., a primate (e.g., human, monkey, gorilla, ape, lemur, etc), a bovine, an equine, a porcine, a canine, or a feline), an insect, a yeast, or a bacteria or is derived from such a protein. Exemplary mammalian H-NOX proteins include wild-type human and rat soluble guanylate cyclase (such as the β1 subunit). Non-limiting examples of H-NOX proteins include wild-type mammalian H-NOX proteins, e.g. H. sapiens, M. musculus, C. familiaris, B. Taurus, C. lupus and R. norvegicus and examples of prokaryotic wild-type H-NOX proteins include T. tengcongensis, V. cholera, V. fischerii, N. punctiforme, D. desulfuricans, L. pneumophila 1, L. pneumophila 2, and C. acetobutylicum. Examples of H-NOX proteins including their NCBI accession numbers may be found in U.S. Pat. Nos. 8,404,631 and 8,404,632, WO 2007/139791 and WO 2007/139767; the contents of each is incorporated herein by reference in its entirety.

Additional H-NOX proteins, H-NOX domains of polymeric H-NOX proteins, and nucleic acids, which may be suitable for use in the pharmaceutical compositions and methods described herein, can be identified using standard methods. For example, standard sequence alignment and/or structure prediction programs can be used to identify additional H-NOX proteins and nucleic acids based on the similarity of their primary and/or predicted protein secondary structure with that of known H-NOX proteins and nucleic acids. For example, the Pfam database uses defined alignment algorithms and Hidden Markov Models (such as Pfam 21.0) to categorize proteins into families, such as the H-NOX protein family (Pfam-A database of protein domain family alignments and Hidden Markov Models, Copyright (C) 1996-2006 The Pfam Consortium; GNU LGPL Free Software Foundation, Inc., 59 Temple Place-Suite 330, Boston, MA 02111-1307, USA). Standard databases such as the swissprot-trembl database (world-wide web at “expasy.org”, Swiss Institute of Bioinformatics Swiss-Prot group CMU-1 rue Michel Servet CH-1211 Geneva 4, Switzerland) can also be used to identify members of the H-NOX protein family. The secondary and/or tertiary structure of an H-NOX protein can be predicted using the default settings of standard structure prediction programs, such as PredictProtein (630 West, 168 Street, BB217, New York, N.Y. 10032, USA). Alternatively, the actual secondary and/or tertiary structure of an H-NOX protein can be determined using standard methods.

In some embodiments, the H-NOX domain has the same amino acid in the corresponding position as any of following distal pocket residues in T. tengcongensis H-NOX: Thr4, Ile5, Thr8, Trp9, Trp67, Asn74, Ile75, Phe78, Phe82, Tyr140, Leu144, or any combination of two or more of the foregoing. In some embodiments, the H-NOX domain has a proline or an arginine in a position corresponding to that of Pro115 or Arg135 of T. tengcongensis H-NOX, respectively, based on sequence alignment of their amino acid sequences. In some embodiments, the H-NOX domain has a histidine that corresponds to His105 of R. norvegicus β1 H-NOX. In some embodiments, the H-NOX domain has or is predicted to have a secondary structure that includes six alpha-helices, followed by two beta-strands, followed by one alpha-helix, followed by two beta-strands. This secondary structure has been reported for H-NOX proteins.

If desired, a newly identified H-NOX protein or H-NOX domain can be tested to determine whether it binds heme using standard methods. The ability of an H-NOX domain to function as an O₂ carrier can be tested by determining whether the H-NOX domain binds O₂ using standard methods, such as those described herein. If desired, one or more of the mutations described herein can be introduced into the H-NOX domain to optimize its characteristics as an O₂ carrier. For example, one or more mutations can be introduced to alter its O₂ dissociation constant, k_{off} for oxygen, rate of heme autoxidation, NO reactivity, NO stability or any combination of two or more of the foregoing. Standard techniques such as those described herein can be used to measure these parameters.

### Mutant H-NOX Proteins

As discussed further herein, an H-NOX protein or an H-NOX domain of a polymeric H-NOX protein may contain one or more mutations, such as a mutation that alters the O₂ dissociation constant, the k_{off} for oxygen, the rate of heme autoxidation, the NO reactivity, the NO stability, or any combination of two or more of the foregoing compared to that of the corresponding wild-type protein. In some embodiments, the invention provides a polymeric H-NOX protein comprising one or more H-NOX domains that may contain one or more mutations, such as a mutation that alters the O₂ dissociation constant, the kor for oxygen, the rate of heme autoxidation, the NO reactivity, the NO stability, or any combination of two or more of the foregoing compared to that of the corresponding wild-type protein. Panels of engineered H-NOX domains may be generated by random mutagenesis followed by empirical screening for requisite or desired dissociation constants, dissociation rates, NO-reactivity, stability, physio-compatibility, or any combination of two or more of the foregoing in view of the teaching provided herein using techniques as described herein and, additionally, as known by the skilled artisan. Alternatively, mutagenesis can be selectively targeted to particular regions or residues such as distal pocket residues apparent from the experimentally determined or predicted three-dimensional structure of an H-NOX protein (see, for example, Boon, E. M. et al. (2005). Nature Chemical Biology 1:53-59, which is hereby incorporated by reference in its entirety, particularly with respect to the sequences of wild-type and mutant H-NOX proteins) or evolutionarily conserved residues identified from sequence alignments (see, for example, Boon E. M. et al. (2005). Nature Chemical Biology 1:53-59, which is hereby incorporated by reference in its entirety, particularly with respect to the sequences of wild-type and mutant H-NOX proteins).

In some embodiments of the invention, the mutant H-NOX protein or mutant H-NOX domain of a polymeric H-NOX protein has a sequence that differs from that of all H-NOX proteins or domains occurring in nature. In various embodiments, the amino acid sequence of the mutant protein is at least about any of 10, 15, 20, 25, 30, 40, 50, 60, 70, 80, 90, 95, 97, 98, 99, or 99.5% identical to that of the corresponding region of an H-NOX protein occurring in nature. In various embodiments, the amino acid sequence of the mutant protein is about 10-20%, 20-30%, 30-40%, 40-50%, 50-60%, 60-70%, 70-80%, 80-90%, 90-95%, 95-99%, or 99.5% identical to that of the corresponding region of an H-NOX protein occurring in nature. In some embodiments, the mutant protein is a protein fragment that contains at least about any of 25, 50, 75, 100, 150, 200, 300, or 400 contiguous amino acids from a full-length protein. In some embodiments, the mutant protein is a protein fragment that contains 25-50, 50-75, 75-100, 100-150, 150-200, 200-300, or 300-400 contiguous amino acids from a full-length protein. Sequence identity can be measured, for example, using sequence analysis software with the default parameters specified therein (e.g., Sequence Analysis Software Package of the Genetics Computer Group, University of Wisconsin Biotechnology Center, 1710 University Avenue, Madison, WI 53705). This software program matches similar sequences by assigning degrees of homology to various amino acids replacements, deletions, and other modifications.

In some embodiments of the invention, the mutant H-NOX protein or mutant H-NOX domain of a polymeric H-NOX protein comprises the insertion of one or more amino acids (e.g., the insertion of 2, 3, 4, 5, 6, 7, 8, 9, or 10 amino acids). In some embodiments of the invention, the mutant H-NOX protein or mutant H-NOX domain comprises the deletion of one or more amino acids (e.g., a deletion of N-terminal, C-terminal, and/or internal residues, such as the deletion of at least about any of 5, 10, 15, 25, 50, 75, 100, 150, 200, 300, or more amino acids or a deletion of 5-10, 10-15, 15-25, 25-50, 50-75, 75-100, 100-150, 150-200, 200-300, or 300-400 amino acids). In some embodiments of the invention, the mutant H-NOX protein or mutant H-NOX domain comprises the replacement of one or more amino acids (e.g., the replacement of 1, 2, 3, 4, 5, 6, 7, 8, 9, or 10 amino acids), or combinations of two or more of the foregoing. In some embodiments, a mutant protein has at least one amino acid alteration compared to a protein occurring in nature. In some embodiments, a mutant nucleic acid sequence encodes a protein that has at least one amino acid alteration compared to a protein occurring in nature. In some embodiments, the nucleic acid is not a degenerate version of a nucleic acid occurring in nature that encodes a protein with an amino acid sequence identical to a protein occurring in nature.

In some embodiments the mutation in the H-NOX protein or H-NOX domain of a polymeric H-NOX protein is an evolutionary conserved mutations (also denoted class I mutations). Examples of class I mutations are listed in Table 1A. In Table 1A, mutations are numbered/annotated according to the sequence of human β1 H-NOX, but are analogous for all H-NOX sequences. Thus, the corresponding position in any other H-NOX protein can be mutated to the indicated residue. For example, Phe4 of human β1 H-NOX can be mutated to a tyrosine since other H-NOX proteins have a tyrosine in this position. The corresponding phenylalanine residue can be mutated to a tyrosine in any other H-NOX protein. In particular embodiments, the one or more mutations are confined to evolutionarily conserved residues. In some embodiments, the one or more mutations may include at least one evolutionarily conserved mutation and at least one non-evolutionarily conserved mutation. If desired, these mutant H-NOX proteins are subjected to empirical screening for NO/O₂ dissociation constants, NO-reactivity, stability, and physio-compatibility in view of the teaching provided herein.

TABLE 1A
Exemplary Class I H-NOX mutations
targeting evolutionary conserved residues

| | | |
|---|---|---|
| F4Y | Q30G | I145Y |
| F4L | E33PI145H | |
| H7G | N61G | K151E |
| A8E | C78H | I157F |
| L9W | A109F | E183F |

In some embodiments, the mutation is a distal pocket mutation, such as mutation of a residue in alpha-helix A, D, E, or G (Pellicena, P. et al. (Aug. 31, 2004). Proc Natl. Acad Sci USA 101 (35): 12854-12859). Exemplary distal pocket mutations (also denoted class II mutations) are listed in Table 1B. In Table 1B, mutations are numbered/annotated according to the sequence of human β1 H-NOX, but are analogous for all H-NOX sequences. Because several substitutions provide viable mutations at each recited residue, the residue at each indicated position can be changed to any other naturally or non-naturally-occurring amino acid (denoted “X”). Such mutations can produce H-NOX proteins with a variety of desired affinity, stability, and reactivity characteristics.

TABLE 1B
Exemplary Class II H-NOX
mutations targeting distal pocket residues

| | | |
|---|---|---|
| V8X | M73X | I145X |
| L9X | F77X | I149X |
| F70X | C78X | |

In particular embodiments, the mutation is a heme distal pocket mutation. As described herein, a crucial molecular determinant that prevents O₂ binding in NO-binding members of the H-NOX family is the lack of a H-bond donor in the distal pocket of the heme. Accordingly, in some embodiments, the mutation alters H-bonding between the H-NOX domain and the ligand within the distal pocket. In some embodiments, the mutation disrupts an H-bond donor of the distal pocket and/or imparts reduced O₂ ligand-binding relative to the corresponding wild-type H-NOX domain. Exemplary distal pocket residues include Thr4, Ile5, Thr8, Trp9, Trp67, Asn74, Ile75, Phe78, Phe82, Tyr140, and Leu144 of T. tengcongensis H-NOX and the corresponding residues in any other H-NOX protein. In some embodiments, the H-NOX protein or H-NOX domain of a polymeric H-NOX protein comprises one or more distal pocket mutations. In some embodiments, the H-NOX protein or H-NOX domain of a polymeric H-NOX protein comprises one, two, three, four, five, six, seven, eight, nine, ten or more than ten distal pocket mutations. In some embodiments, the distal pocket mutation corresponds to a L144F mutation of T. tengcongensis H-NOX. In some embodiments, the distal pocket mutation is a L144F mutation of T. tengcongensis H-NOX. In some embodiments, H-NOX protein or the H-NOX domain of a polymeric H-NOX protein comprises two distal pocket mutations. In some embodiments, the H-NOX protein or H-NOX domain of a polymeric H-NOX protein corresponds to a W9F/L144F mutation of T. tengcongensis H-NOX. In some embodiments, the H-NOX protein or H-NOX domain of a polymeric H-NOX protein is a W9F/L144F mutation of T. tengcongensis H-NOX.

Residues that are not in the distal pocket can also affect the three-dimensional structure of the heme group; this structure in turn affects the binding of O₂ and NO to iron in the heme group. Accordingly, in some embodiments, the H-NOX protein or H-NOX domain of a polymeric H-NOX protein has one or more mutations outside of the distal pocket. Examples of residues that can be mutated but are not in the distal pocket include Pro115 and Arg135 of T. tengcongensis H-NOX. In some embodiments, the mutation is in the proximal pocket which includes His105 as a residue that ligates to the heme iron.

In some embodiments when two or more mutations are present; at least one mutation is in the distal pocket, and at least one mutation is outside of the distal pocket (e.g., a mutation in the proximal pocket). In some embodiments, all the mutations are in the distal pocket.

To reduce the immunogenicity of H-NOX protein or H-NOX domains derived from sources other than humans, amino acids in an H-NOX protein or H-NOX domain can be mutated to the corresponding amino acids in a human H-NOX. For example, one or more amino acids on the surface of the tertiary structure of a non-human H-NOX protein or H-NOX domain can be mutated to the corresponding amino acid in a human H-NOX protein or H-NOX domain. In some variations, mutation of one or more surface amino acids may be combined with mutation of two or more distal pocket residues, mutation of one or more residues outside of the distal pocket (e.g., a mutation in the proximal pocket), or combinations of two or more of the foregoing.

The invention also relates to any combination of mutation described herein, such as double, triple, or higher multiple mutations. For example, combinations of any of the mutations described herein can be made in the same H-NOX protein. Note that mutations in equivalent positions in other mammalian or non-mammalian H-NOX proteins are also encompassed by this invention. Exemplary mutant H-NOX proteins or mutant H-NOX domains comprise one or more mutations that impart altered O₂ or NO ligand-binding relative to the corresponding wild-type H-NOX domain and are operative as a physiologically compatible mammalian O₂ blood gas carrier.

The residue number for a mutation indicates the position in the sequence of the particular H-NOX protein being described. For example, T. tengcongensis 15A refers to the replacement of isoleucine by alanine at the fifth position in T. tengcongensis H-NOX. The same isoleucine to alanine mutation can be made in the corresponding residue in any other H-NOX protein or H-NOX domain (this residue may or may not be the fifth residue in the sequence of other H-NOX proteins). Since the amino acid sequences of mammalian B1 H-NOX domains differ by at most two amino acids, mutations that produce desirable mutant H-NOX proteins or H-NOX domains when introduced into wild-type rat β1 H-NOX proteins are also expected to produce desirable mutant H-NOX proteins or H-NOX domains when introduced into wild-type β1 H-NOX proteins or H-NOX domains from other mammals, such as humans.

In some embodiments, the H-NOX protein is a trimer comprising three T. tengcongensis L144F H-NOX domains and three foldon domains. In some embodiments, the H-NOX protein is a trimer comprising three T. tengcongensis W9F/L144F H-NOX domains and three foldon domains. In some embodiments, the H-NOX protein is a trimer comprising three T. tengcongensis wildtype H-NOX domains and three foldon domains.

### Modifications to H-NOX Proteins

Any of the wild-type or mutant H-NOX proteins, including polymeric H-NOX proteins, can be modified and/or formulated using standard methods to enhance therapeutic or industrial applications. For example, and particularly as applied to heterologous engineered H-NOX proteins, a variety of methods are known in the art for insulating such agents from immune surveillance, including crosslinking, PEGylation, carbohydrate decoration, etc. (e.g., Rohlfs, R. J. et al. (May 15, 1998). J. Biol. Chem. 273 (20): 12128-12134; Migita, R. et al. (June 1997). J. Appl. Physiol. 82 (6): 1995-2002; Vandegriff, K. D. et al. (Aug. 15, 2004). Biochem J. 382 (Pt 1): 183-189, which are each hereby incorporated by reference in their entireties, particularly with respect to the modification of proteins) as well as other techniques known to the skilled artisan. Fusing an H-NOX protein, including a polymeric H-NOX protein, with a human protein such as human serum albumin can increase the serum half-life, viscosity, and colloidal oncotic pressure. In some embodiments, an H-NOX protein is modified during or after its synthesis to decrease its immunogenicity and/or to increase its plasma retention time. H-NOX proteins can also be encapsulated (such as encapsulation within liposomes or nanoparticles).

In some embodiments, the H-NOX protein comprises one of more tags; e.g. to assist in purification of the H-NOX protein. Examples of tags include, but are not limited to His₆, FLAG, GST, and MBP. In some embodiments, the H-NOX protein comprises one of more His₆ tags. The one or more His₆ tags may be removed prior to use of the polymeric H-NOX protein; e.g. by treatment with an exopeptidase. In some embodiments, the H-NOX protein is a trimer comprising three T. tengcongensis L144F H-NOX domains, three foldon domains, and three His₆ tags. In some embodiments, the H-NOX protein is a trimer comprising three T. tengcongensis W9F/L144F H-NOX domains, three foldon domains, and three His₆ tags. In some embodiments, the H-NOX protein is a trimer comprising three T. tengcongensis wildtype H-NOX domains, three foldon domains, and three His₆ tags.

In some embodiments, the H-NOX protein comprises one or more polyethylene glycol (PEG) molecules (i.e., is PEGylated). In some embodiments, the H-NOX protein is a trimer comprising three T. tengcongensis L144F H-NOX domains, three foldon domains, and one or more polyethylene glycol molecules (PEGylated trimer Tt H-NOX L144F). In some embodiments, the H-NOX protein is a trimer comprising three T. tengcongensis W9F/L144F H-NOX domains, three foldon domains, and one or more polyethylene glycol molecules. In some embodiments, the H-NOX protein is a trimer comprising three T. tengcongensis wildtype H-NOX domains, three foldon domains, and one or more polyethylene glycol molecules. In some embodiments, the molecular weight of the PEG is between about 1 kDa and about 50 kDa. In some embodiments, the molecular weight of the PED is between about any of 1 kDa and 50 kDa, 1 kDa and 40 kDa, 1 kDa and 30 kDa, 1 kDa and 25 kDa, 1 kDa and 20 kDa, 1 kDa and 15 kDa, 1 kDa and 10 kDa, 1 kDa and 5 kDa, 5 kDa and 50 kDa, 5 kDa and 40 kDa, 5 kDa and 30 kDa, 5 kDa and 25 kDa, 5 kDa and 20 kDa, 5 kDa and 15 kDa, 5 kDa and 10 kDa, 10 kDa and 50 kDa, 10 kDa and 40 kDa, 10 kDa and 30 kDa, 10 kDa and 25 kDa, 10 kDa and 20 kDa, 10 kDa and 15 kDa, 15 kDa and 50 kDa, 15 kDa and 40 kDa, 15 kDa and 35 kDa, 15 kDa and 30 kDa, 15 kDa and 25 kDa, 15 kDa and 20 kDa, 20 kDa and 50 kDa, 20 kDa and 40 kDa, 20 kDa and 30 kDa, 20 kDa and 25 kDa, 25 kDa and 50 kDa, 25 kDa and 40 kDa, 25 kDa and 30 kDa, 30 kDa and 50 kDa, 30 kDa and 40 kDa, or 40 kDa and 50 kDa. In some embodiments, the H-NOX protein comprises any one of more than about 1, 2, 3, 4, 5, 6, 7, 8, 9, 10, 15, 20, 25, 30, 35, 40, 45, or 50 PEG molecules per H-NOX monomer or any number therebetween. In some embodiments, the H-NOX protein comprises an average of 1, 2, 3, 4, 5, 6, 7, 8, 9, 10, 15, 20, 25, 30, 35, 40, 45, or 50 PEG molecules per H-NOX monomer or any number therebetween.

### Polymerization Domains

In some aspects, the invention provides polymeric H-NOX proteins comprising two or more H-NOX domains and one or more polymerization domains. Polymerization domains are used to link two or more H-NOX domains to form a polymeric H-NOX protein. One or more polymerization domains may be used to produce dimers, trimers, tetramers, pentamers, etc. of H-NOX proteins. Polymerization domains are known in the art, such as: the foldon of T4 bacteriophage fibritin, Arc, POZ, coiled coil domains (including GCN4, leucine zippers, Velcro), uteroglobin, collagen, 3-stranded coiled colis (matrilin-1), thrombosporins, TRPV1-C, P53, Mnt, avadin, streptavidin, Bcr-Abl, COMP, verotoxin subunit B, CamKII, RCK, and domains from N ethylmaleimide-sensitive fusion protein, STM3548, KaiC, TyrR, Hcp1, CcmK4, GP41, anthrax protective antigen, aerolysin, a-hemolysin, C4b-binding protein, Mi-CK, arylsurfatase A, and viral capsid proteins. The polymerization domains may be covalently or non-covalently linked to the H-NOX domains. In some embodiments, a polymerization domain is linked to an H-NOX domain to form a monomer subunit such that the polymerization domains from a plurality of monomer subunits associate to form a polymeric H-NOX domain. In some embodiments, the C-terminus of an H-NOX domain is linked to the N-terminus of a polymerization domain. In other embodiments, the N-terminus of an H-NOX domain is linked to the N-terminus of a polymerization domain. In yet other embodiments, the C-terminus of an H-NOX domain is linked to the C-terminus of a polymerization domain. In some embodiments, the N-terminus of an H-NOX domain is linked to the C-terminus of a polymerization domain.

Linkers may be used to join a polymerization domain to an H-NOX domain; for example, for example, amino acid linkers. In some embodiments, a linker comprising any one of one, two, three, four, five, six, seven, eight, nine, ten or more than ten amino acids may be placed betweent the polymerization domain and the H-NOX domain. Exemplary linkers include but are not limited to Gly-Ser-Gly and Arg-Gly-Ser linkers.

### Bacteriophage T4 Fibritin Trimerization Domain

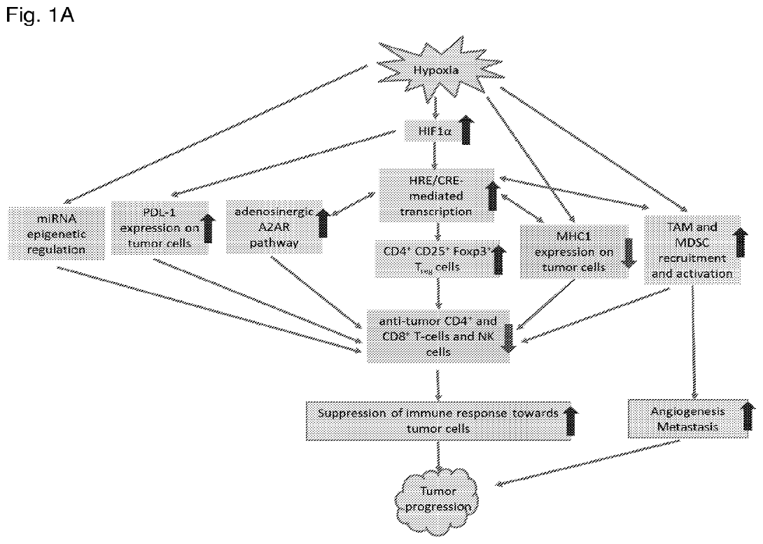

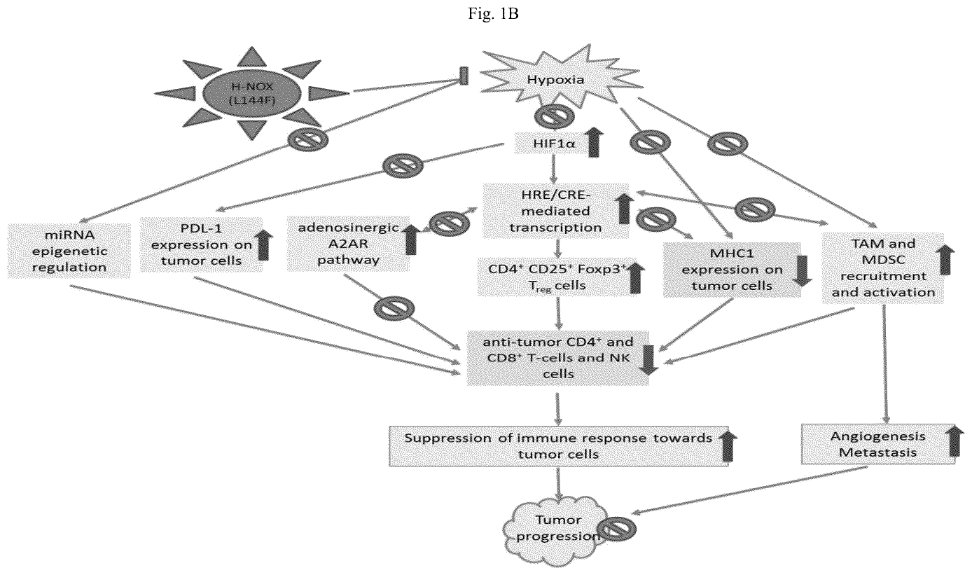

An exemplary polymerization domain is the foldon domain of bacteriophage T4. The wac gene from the bacteriophage T4 encodes the fibritin protein, a 486 amino acid protein with a C-terminal trimerization domain (residues 457-483) (Efimov, V. P. et al. (1994) J Mol Biol 242:470-486). The domain is able to trimerize fibritin both in vitro and in vivo (Boudko, S. P. et al. (2002) Eur J Biochem 269:833-841; Letarov, A. V., et al., (1999) Biochemistry (Mosc) 64:817-823; Tao, Y., et al., (1997) Structure 5:789-798). The isolated 27 residue trimerization domain, often referred to as the “foldon domain,” has been used to construct chimeric trimers in a number of different proteins (including HIV envelope glycoproteins (Yang, X. et al., (2002) J Virol 76:4634-4642), adenoviral adhesins (Papanikolopoulou, K., et al., (2004) J Biol Chem 279:8991-8998; Papanikolopoulou, K. et al. (2004) J Mol Biol 342:219-227), collagen (Zhang, C., et al. (2009) Biotechnol Prog 25:1660-1668), phage P22 gp26 (Bhardwaj, A., et al. (2008) Protein Sci 17:1475-1485), and rabies virus glycoprotein (Sissoeff, L., et al. (2005) J Gen Virol 86:2543-2552). An exemplary sequence of the foldon domain is shown in FIG. 1 and provided by SEQ ID NO:4.

The isolated foldon domain folds into a single β-hairpin structure and trimerizes into a β-propeller structure involving three hairpins (Guthe, S. et al. (2004) J Mol Biol 337:905-915). The structure of the foldon domain alone has been determined by NMR (Guthe, S. et al. (2004) J Mol Biol 337:905-915) and the structures of several proteins trimerized with the foldon domain have been solved by X-ray crystallography (Papanikolopoulou, K., et al., (2004) J Biol Chem 279:8991-8998; Stetefeld, J. et al. (2003) Structure 11:339-346; Yokoi, N. et al. (2010) Small 6:1873-1879). The domain folds and trimerizes rapidly reducing the opportunity for misfolding intermediates or off-pathway oligomerization products (Guthe, S. et al. (2004) J Mol Biol 337:905-915). The foldon domain is very stable, able to maintain tertiary structure and oligomerization in >10% SDS, 6.0M guanidine hydrochloride, or 80° C. (Bhardwaj, A., et al. (2008) Protein Sci 17:1475-1485; Bhardwaj, A., et al. (2007) J Mol Biol 371:374-387) and can improve the stability of sequences fused to the foldon domain (Du, C. et al. (2008) Appl Microbiol Biotechnol 79:195-202.

In some embodiments, the C-terminus of an H-NOX domain is linked to the N-terminus of a foldon domain. In other embodiments, the N-terminus of an H-NOX domain is linked to the N-terminus of a foldon domain. In yet other embodiments, the C-terminus of an H-NOX domain is linked to the C-terminus of a foldon domain. In some embodiments, the N-terminus of an H-NOX domain is linked to the C-terminus of a foldon domain.

In some embodiments, linkers are be used to join a foldon domain to an H-NOX domain. In some embodiments, a linker comprising any one of one, two, three, four, five, six, seven, eight, nine, ten or more than ten amino acids may be placed between the polymerization domain and the H-NOX domain. Exemplary linkers include but are not limited to Gly-Ser-Gly and Arg-Gly-Ser linkers. In some embodiments, the invention provides a trimeric H-NOX protein comprising from N-terminus to C-terminus: a T. tengcongensis H-NOX domain, a Gly-Ser-Gly amino acid linker, and a foldon domain. In some embodiments, the invention provides a trimeric H-NOX protein comprising from N-terminus to C-terminus: a T. tengcongensis H-NOX domain, a Gly-Ser-Gly amino acid linker, a foldon domain, an Arg-Gly-Ser amino acid linker, and a His₆ tag. In some embodiments, the T. tengcongensis H-NOX domain comprises an L144F mutation. In some embodiments, the T. tengcongensis H-NOX domain comprises a W9F mutation and a L144F mutation. In some embodiments, the T. tengcongensis H-NOX domain is a wild-type H-NOX domain.

### Monomeric H-NOX Domain Subunits

In one aspect, the invention provides recombinant monomeric H-NOX proteins (i.e. monomeric H-NOX subunits of polymeric H-NOX proteins) that can associate to form polymeric H-NOX proteins. In some embodiments, the invention provides recombinant H-NOX proteins comprising an H-NOX domain as described herein and a polymerization domain. The H-NOX domain and the polymerization domain may be covalently linked or noncovalently linked. In some embodiments, the C-terminus of an H-NOX domain of the recombinant monomeric H-NOX protein is linked to the N-terminus of a polymerization domain. In other embodiments, the N-terminus of an H-NOX domain of the recombinant monomeric H-NOX protein is linked to the N-terminus of a polymerization domain. In yet other embodiments, the C-terminus of an H-NOX domain of the recombinant monomeric H-NOX protein is linked to the C-terminus of a polymerization domain. In some embodiments, the N-terminus of an H-NOX domain of the recombinant monomeric H-NOX protein is linked to the C-terminus of a polymerization domain. In some embodiments, the recombinant monomeric H-NOX protein does not comprise a guanylyl cyclase domain.

In some embodiments, the monomeric H-NOX protein comprises a wild-type H-NOX domain. In some embodiments of the invention, the monomeric H-NOX protein comprises one of more mutations in the H-NOX domain. In some embodiments, the one or more mutations alter the O₂ dissociation constant, the k_{off} for oxygen, the rate of heme autooxidation, the NO reactivity, the NO stability or any combination of two or more of the foregoing compared to that of the corresponding wild-type H-NOX domain. In some embodiments, the mutation is a distal pocket mutation. In some embodiments, the mutation comprises a mutation that is not in the distal pocket. In some embodiments, the distal pocket mutation corresponds to a L144 mutation of T. tengcongensis (e.g. a L144F mutation). In some embodiments, the recombinant monomeric H-NOX protein comprises two distal pocket mutations corresponding to a W9 and a L144 mutation of T. tengcongensis (e.g. a W9F/L144F mutation).

In some aspects, the invention provides recombinant monomeric H-NOX proteins that associate to form trimeric H-NOX proteins. In some embodiments, the recombinant H-NOX protein comprises an H-NOX domain and a trimerization domain. In some embodiments, the trimerization domain is a foldon domain as discussed herein. In some embodiments, the H-NOX domain is a T. tengcongensis H-NOX domain. In some embodiments the C-terminus of the T. tengcongensis H-NOX domain is covalently linked to the N-terminus of the foldon domain. In some embodiments the C-terminus of the T. tengcongensis H-NOX domain is covalently linked to the C-terminus of the foldon domain. In some embodiments, the T. tengcongensis domain is an L144F H-NOX domain. In some embodiments, the T. tengcongensis domain is a W9F/L144F H-NOX domain. In some embodiments, the T. tengcongensis domain is a wild-type H-NOX domain.

In some embodiments, the H-NOX domain is covalently linked to the polymerization domain using an amino acid linker sequence. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence. In some embodiments, the polymeric H-NOX protein is a trimeric H-NOX protein comprising three H-NOX domains and three trimerization sequences wherein the H-NOX domain is covalently linked to the trimerization domain via an amino acid linker sequence. In some embodiments, the monomeric H-NOX protein comprises the following from the N-terminus to the C-terminus: an L144F T. tengcongensis H-NOX domain, a Gly-Ser-Gly amino acid linker sequence, and a foldon domain. In some embodiments, the monomeric H-NOX protein comprises the following from the N-terminus to the C-terminus: a W9F/L144F T. tengcongensis H-NOX domain, a Gly-Ser-Gly amino acid linker sequence, and a foldon domain. In some embodiments, the monomeric H-NOX protein comprises the following from the N-terminus to the C-terminus: a wild-type T. tengcongensis H-NOX domain, a Gly-Ser-Gly amino acid linker sequence, and a foldon domain.

In some embodiments, the recombinant monomeric H-NOX protein comprises a tag; e.g., a His₆, a FLAG, a GST, or an MBP tag. In some embodiments, the recombinant monomeric H-NOX protein comprises a His₆ tag. In some embodiments, the recombinant monomeric H-NOX protein does not comprise a tag. In some embodiments, the tag (e.g. a His₆ tag) is covalently linked to the polymerization domain using an amino acid spacer sequence. In some embodiments, the amino acid linker sequence is one, two, three, four, five, six, seven, eight, nine, ten or more than ten amino acids in length. Exemplary amino acid linker sequences include but are not limited to a Gly-Ser-Gly sequence and an Arg-Gly-Ser sequence. In some embodiments, the polymeric H-NOX protein is a trimeric H-NOX protein comprising three H-NOX domains, three trimerization sequences, and three His₆ tags, wherein the H-NOX domain is covalently linked to the trimerization domain via an amino acid linker sequence and the trimerization domain is covalently linked to the His₆ tag via an amino acid linker sequence. In some embodiments, the monomeric H-NOX protein comprises the following from the N-terminus to the C-terminus: an L144F T. tengcongensis H-NOX domain, a Gly-Ser-Gly amino acid linker sequence, a foldon domain, an Arg-Gly-Ser linker sequence, and a His₆ tag. In some embodiments, the monomeric H-NOX protein comprises the following from the N-terminus to the C-terminus: a W9F/L144F T. tengcongensis H-NOX domain, a Gly-Ser-Gly amino acid linker sequence, a foldon domain, an Arg-Gly-Ser linker sequence, and a His₆ tag. In some embodiments, the monomeric H-NOX protein comprises the following from the N-terminus to the C-terminus: a wild-type T. tengcongensis H-NOX domain, a Gly-Ser-Gly amino acid linker sequence, a foldon domain, an Arg-Gly-Ser linker sequence, and a His₆ tag.

In some embodiments the recombinant monomeric H-NOX protein comprises the amino acid sequence of SEQ ID NO:6 or SEQ ID NO:8.

### Characteristics of Wild-Type and Mutant H-NOX Proteins

The present invention provides the use of O₂ carrier polypeptides for use in enhancing tumor immunogenicity; for example, by inhibiting the immune suppressive activities associated with tumor hypoxia. A non-limiting exemplary family of O₂ carrier polypeptides is the H-NOX family of O₂ carrier polypeptides. As described herein, a large number of diverse H-NOX mutant proteins, including polymeric H-NOX proteins, providing ranges of NO and O₂ dissociation constants, O₂ k_{off}, NO reactivity, and stability have been generated. To provide operative blood gas carriers, the H-NOX proteins may be used to functionally replace or supplement endogenous O₂ carriers, such as hemoglobin. In some embodiments, H-NOX proteins such as polymeric H-NOX proteins, are used to deliver O₂ to hypoxic tumor tissue (e.g. a glioblastoma) as an adjuvant to radiation therapy or chemotherapy. Accordingly, in some embodiments, an H-NOX protein has a similar or improved O₂ association rate, O₂ dissociation rate, dissociation constant for O₂ binding, NO stability, NO reactivity, autoxidation rate, plasma retention time, or any combination of two or more of the foregoing compared to an endogenous O₂ carrier, such as hemoglobin. In some embodiments, the H-NOX protein is a polymeric H-NOX protein. In some embodiments, the polymeric H-NOX protein is a trimeric H-NOX protein comprising three monomers, each monomer comprising a T. tengcongensis L144F H-NOX domain and a foldon domain. In some embodiments, the polymeric H-NOX protein is a trimeric H-NOX protein comprising three monomers, each monomer comprising a T. tengcongensis W9F/L144F H-NOX domain and a foldon domain. In some embodiments, the polymeric H-NOX protein is a trimeric H-NOX protein comprising three monomers, each monomer comprising a T. tengcongensis L144F H-NOX domain and a foldon domain.

In various embodiments, the k_{off} for O₂ for an H-NOX protein, including a polymeric H-NOX protein, is between about 0.01 to about 200 s⁻¹ at 20° C., such as about 0.1 to about 200 s⁻¹, about 0.1 to 100 s⁻¹, about 1.0 to about 16.0 s⁻¹, about 1.35 to about 23.4 s⁻¹, about 1.34 to about 18 s⁻¹, about 1.35 to about 14.5 s⁻¹, about 0.21 to about 23.4 s⁻¹, about 1.35 to about 2.9 s⁻¹, about 2 to about 3 s⁻¹, about 5 to about 15 s⁻¹, or about 0.1 to about 1 s⁻¹. In some embodiments, the H-NOX protein has a k_{off} for oxygen that is less than or equal to about 0.65 s⁻¹ at 20° C. (such as between about 0.21 s⁻¹ to about 0.65 s⁻¹ at 20° C.).

In various embodiments, the kₒₙ for O₂ for an H-NOX protein, including a polymeric H-NOX protein, is between about 0.14 to about 60 μM⁻¹ s⁻¹ at 20° C., such as about 6 to about 60 μM⁻¹ s⁻¹, about 6 to 12 μM⁻¹ s⁻¹, about 15 to about 60 μM⁻¹ s⁻¹, about 5 to about 18 μM⁻s⁻¹, or about 6 to about 15 μM⁻¹ s⁻¹.

In various embodiments, the kinetic or calculated K_{D} for O₂ binding by an H-NOX protein, including a polymeric H-NOX protein, is between about 1 nM to 1 mM, about 1 μM to about 10 μM, or about 10 μM to about 50 μM. In some embodiments the calculated K_{D} for O₂ binding is any one of about 2 nM to about 2 μM, about 2 μM to about 1 mM, about 100 nM to about 1 μM, about 9 μM to about 5 μM, about 100 μM to about 1 mM, about 50 nM to about 10 μM, about 2 nM to about 50 μM, about 100 nM to about 1.9 μM, about 150 nM to about 1 μM, or about 100 nM to about 255 nM, about 20 nM to about 2 μM, 20 nM to about 75 nM, about 1 μM to about 2 μM, about 2 μM to about 10 μM, about 2 μM to about 9 μM, or about 100 nM to 500 nM at 20° C. In some embodiments, the kinetic or calculated K_{D} for O₂ binding is less than about any of 100 nM, 80 nM, 50 nM, 30 nM, 25 nM, 20 nM, or 10 nM at 20° C.

In various embodiments, the kinetic or calculated K_{D} for O₂ binding by an H-NOX protein, including a polymeric H-NOX protein, is within about 0.01 to about 100-fold of that of hemoglobin under the same conditions (such as at 20° C.), such as between about 0.1 to about 10-fold or between about 0.5 to about 2-fold of that of hemoglobin under the same conditions (such as at 20° C.). In various embodiments, the kinetic or calculated K_{D} for NO binding by an H-NOX protein is within about 0.01 to about 100-fold of that of hemoglobin under the same conditions (such as at 20° C.), such as between about 0.1 to about 10-fold or between about 0.5 to about 2-fold of that of hemoglobin under the same conditions (such as at 20° C.).

In some embodiments, less than about any of 50, 40, 30, 10, or 5% of an H-NOX protein, including a polymeric H-NOX protein, is oxidized after incubation for about any of 1, 2, 4, 6, 8, 10, 15, or 20 hours at 20° C.

In various embodiments, the NO reactivity of an H-NOX protein, including a polymeric H-NOX protein, is less than about 700 s⁻¹ at 20° C., such as less than about 600 s⁻¹, 500 s⁻¹, 400 s⁻¹, 300 s⁻¹, 200 s⁻¹, 100 s⁻¹, 75 s⁻¹, 50 s⁻¹, 25 s⁻¹, 20 s⁻¹, 10 s⁻¹, 50 s⁻¹, 3 s⁻¹, 2 s⁻¹, 1.8 s⁻¹, 1.5 s⁻¹, 1.2 s⁻¹, 1.0 s⁻¹, 0.8 s⁻¹, 0.7 s⁻¹, or 0.6 s⁻¹ at 20° C. In various embodiments, the NO reactivity of an H-NOX protein is between about 0.1 to about 600 s⁻¹ at 20° C., such as between about 0.5 to about 400 s⁻¹, about 0.5 to about 100 s⁻¹, about 0.5 to about 50 s⁻¹, about 0.5 to about 10 s⁻¹, about 1 to about 5 s⁻¹, or about 0.5 to about 2.1 s⁻¹ at 20° C. In various embodiments, the reactivity of an H-NOX protein is at least about 10, 100, 1,000, or 10,000 fold lower than that of hemoglobin under the same conditions, such as at 20° C.

In various embodiments, the rate of heme autoxidation of an H-NOX protein, including a polymeric H-NOX protein, is less than about 1.0 h⁻¹ at 37° C., such as less than about any of 0.9 h⁻¹, 0.8 h⁻¹, 0.7 h⁻¹, 0.6 h⁻¹, 0.5 h⁻¹, 0.4 h⁻¹, 0.3 h⁻¹, 0.2 h⁻¹, 0.1 h⁻¹, or 0.05 h⁻¹ at 37 C. In various embodiments, the rate of heme autoxidation of an H-NOX protein is between about 0.006 to about 5.0 h⁻¹ at 37° C., such as about 0.006 to about 1.0 h⁻¹, 0.006 to about 0.9 h⁻¹, or about 0.06 to about 0.5 h⁻¹ at 37° C.

In various embodiments, a mutant H-NOX protein, including a polymeric H-NOX protein, has (a) an O₂ or NO dissociation constant, association rate (kₒₙ for O₂ or NO), or dissociation rate (k_{off} for O₂ or NO) within 2 orders of magnitude of that of hemoglobin, (b) has an NO affinity weaker (e.g., at least about 10-fold, 100-fold, or 1000-fold weaker) than that of sGC β1, respectively, (c) an NO reactivity with bound O₂ at least 1000-fold less than hemoglobin, (d) an in vivo plasma retention time at least 2, 10, 100, or 1000-fold higher than that of hemoglobin, or (e) any combination of two or more of the foregoing.

Exemplary suitable O₂ carriers provide dissociation constants within two orders of magnitude of that of hemoglobin, i.e. between about 0.01 and 100-fold, such as between about 0.1 and 10-fold, or between about 0.5 and 2-fold of that of hemoglobin. A variety of established techniques may be used to quantify dissociation constants, such as the techniques described herein (Boon, E. M. et al. (2005). Nature Chem. Biol. 1:53-59; Boon, E. M. et al. (October 2005). Curr. Opin. Chem. Biol. 9 (5): 441-446; Boon, E. M. et al. (2005). J. Inorg. Biochem. 99 (4): 892-902), Vandegriff, K. D. et al. (Aug. 15, 2004). Biochem J. 382 (Pt 1): 183-189, which are each hereby incorporated by reference in their entireties, particularly with respect to the measurement of dissociation constants), as well as those known to the skilled artisan. Exemplary O₂ carriers provide low or minimized NO reactivity of the H-NOX protein with bound O₂, such as an NO reactivity lower than that of hemoglobin. In some embodiments, the NO reactivity is much lower, such as at least about 10, 100, 1,000, or 10,000-fold lower than that of hemoglobin. A variety of established techniques may be used to quantify NO reactivity (Boon, E. M. et al. (2005). Nature Chem. Biol. 1:53-59; Boon, E. M. et al. (October 2005). Curr. Opin. Chem. Biol. 9 (5): 441-446; Boon, E. M. et al. (2005). J. Inorg. Biochem. 99 (4): 892-902), Vandegriff, K. D. et al. (Aug. 15, 2004). Biochem J. 382 (Pt 1): 183-189, which are each hereby incorporated by reference in their entireties, particularly with respect to the measurement of NO reactivity) as well as those known to the skilled artisan. Because wild-type T. tengcongensis H-NOX has such a low NO reactivity, other wild-type H-NOX proteins and mutant H-NOX proteins may have a similar low NO reactivity. For example, T. tengcongensis H-NOX Y140H has an NO reactivity similar to that of wild-type T. tengcongensis H-NOX.

In addition, suitable O₂ carriers provide high or maximized stability, particularly in vivo stability. A variety of stability metrics may be used, such as oxidative stability (e.g., stability to autoxidation or oxidation by NO), temperature stability, and in vivo stability. A variety of established techniques may be used to quantify stability, such as the techniques described herein (Boon, E. M. et al. (2005). Nature Chem. Biol. 1:53-59; Boon, E. M. et al. (October 2005). Curr. Opin. Chem. Biol. 9 (5): 441-446; Boon, E. M. et al. (2005). J. Inorg. Biochem. 99 (4): 892-902), as well as those known to the skilled artisan. For in vivo stability in plasma, blood, or tissue, exemplary metrics of stability include retention time, rate of clearance, and half-life. H-NOX proteins from thermophilic organisms are expected to be stable at high temperatures. In various embodiments, the plasma retention times are at least about 2-, 10-, 100-, or 1000-fold greater than that of hemoglobin (e.g. Bobofchak, K. M. et al. (August 2003). Am. J. Physiol. Heart Circ. Physiol. 285 (2):H549-H561). As will be appreciated by the skilled artisan, hemoglobin-based blood substitutes are limited by the rapid clearance of cell-free hemoglobin from plasma due the presence of receptors for hemoglobin that remove cell-free hemoglobin from plasma. Since there are no receptors for H-NOX proteins in plasma, wild-type and mutant H-NOX proteins are expected to have a longer plasma retention time than that of hemoglobin. If desired, the plasma retention time can be increased by PEGylating or crosslinking an H-NOX protein or fusing an H-NOX protein with another protein using standard methods (such as those described herein and those known to the skilled artisan).

In various embodiments, the H-NOX protein, including a polymeric H-NOX protein, has an O₂ dissociation constant between about 1 nM to about 1 mM at 20° C. and a NO reactivity at least about 10-fold lower than that of hemoglobin under the same conditions, such as at 20° C. In some embodiments, the H-NOX protein has an O₂ dissociation constant between about 1 nM to about 1 mM at 20° C. and a NO reactivity less than about 700 s⁻¹ at 20° C. (e.g., less than about 600 s⁻¹, 500 s⁻¹, 100 s⁻¹, 20 s⁻¹, or 1.8 s⁻¹ at 20° C.). In some embodiments, the H-NOX protein has an O₂ dissociation constant within 2 orders of magnitude of that of hemoglobin and a NO reactivity at least about 10-fold lower than that of hemoglobin under the same conditions, such as at 20° C. In some embodiments, the H-NOX protein has a k_{off} for oxygen between about 0.01 to about 200 s⁻¹ at 20° C. and an NO reactivity at least about 10-fold lower than that of hemoglobin under the same conditions, such as at 20° C. In some embodiments, the H-NOX protein has a kor for oxygen that is less than about 0.65 s⁻¹ at 20° C. (such as between about 0.21 s⁻¹ to about 0.64 s⁻¹ at 20° C.) and a NO reactivity at least about 10-fold lower than that of hemoglobin under the same conditions, such as at 20° C. In some embodiments of the invention, the O₂ dissociation constant of the H-NOX protein is between about 1 nM to about 1 μM (1000 nM), about 1 μM to about 10 μM, or about 10 μM to about 50 μM. In particular embodiments, the O₂ dissociation constant of the H-NOX protein is between about 2 nM to about 50 μM, about 50 nM to about 10 μM, about 100 nM to about 1.9 μM, about 150 nM to about 1 μM, or about 100 nM to about 255 nM at 20° C. In various embodiments, the O₂ dissociation constant of the H-NOX protein is less than about 80 nM at 20° C., such as between about 20 nM to about 75 nM at 20° C. In some embodiments, the NO reactivity of the H-NOX protein is at least about 100-fold lower or about 1,000 fold lower than that of hemoglobin, under the same conditions, such as at 20° C. In some embodiments, the NO reactivity of the H-NOX protein is less than about 700 s⁻¹ at 20° C., such as less than about 600 s⁻¹, 500 s⁻¹, 400 s⁻¹, 300 s⁻¹, 200 s⁻¹, 100 s⁻¹, 75 s⁻¹, 50 s⁻¹, 25 s⁻¹, 20 s⁻¹, 10 s⁻¹, 50 s⁻¹, 3 s⁻¹, 2 s⁻¹, 1.8 s⁻¹, 1.5 s⁻¹, 1.2 s⁻¹, 1.0 s⁻¹, 0.8 s⁻¹, 0.7 s⁻¹, or 0.6 s⁻¹ at 20° C. In some embodiments, the k_{off} for oxygen of the H-NOX protein is between 0.01 to 200 s⁻¹ at 20° C., such as about 0.1 to about 200 s⁻¹, about 0.1 to 100 s⁻¹, about 1.35 to about 23.4 s⁻¹, about 1.34 to about 18 s⁻¹, about 1.35 to about 14.5 s⁻¹, about 0.21 to about 23.4 s⁻¹, about 2 to about 3 s⁻¹, about 5 to about 15 s⁻¹, or about 0.1 to about 1 s⁻¹. In some embodiments, the O₂ dissociation constant of the H-NOX protein is between about 100 nM to about 1.9 μM at 20° C., and the k_{off} for oxygen of the H-NOX protein is between about 1.35 s⁻¹ to about 14.5 s⁻¹ at 20° C. In some embodiments, the rate of heme autoxidation of the H-NOX protein is less than about 1 h⁻¹ at 37° C., such as less than about any of 0.9 h⁻¹, 0.8 h⁻¹, 0.7 h⁻¹, 0.6 h⁻¹, 0.5 h⁻¹, 0.4 h⁻¹, 0.3 h⁻¹, 0.2 h⁻¹, or 0.1 h⁻¹. In some embodiments, the k_{off} for oxygen of the H-NOX protein is between about 1.35 s⁻¹ to about 14.5 s⁻¹ at 20° C., and the rate of heme autoxidation of the H-NOX protein is less than about 1 h⁻¹ at 37° C. In some embodiments, the k_{off} for oxygen of the H-NOX protein is between about 1.35 s⁻¹ to about 14.5 s⁻¹ at 20° C., and the NO reactivity of the H-NOX protein is less than about 700 s⁻¹ at 20° C. (e.g., less than about 600 s⁻¹, 500 s⁻¹, 100 s⁻¹, 20 s⁻¹, or 1.8 s⁻¹ at 20° C.). In some embodiments, the rate of heme autoxidation of the H-NOX protein is less than about 1 h⁻¹ at 37° C., and the NO reactivity of the H-NOX protein is less than about 700 s⁻¹ at 20° C. (e.g., less than about 600 s⁻¹, 500 s⁻¹, 100 s⁻¹, 20 s⁻¹, or 1.8 s⁻¹ at 20° C.).

In some embodiments, the viscosity of the H-NOX protein solution, including a polymeric H-NOX protein solution, is between 1 and 4 centipoise (cP). In some embodiments, the colloid oncotic pressure of the H-NOX protein solution is between 20 and 50 mm Hg.

Measurement of O₂ and/or NO Binding

One skilled in the art can readily determine the oxygen and nitric oxide binding characteristics of any H-NOX protein including a polymeric H-NOX protein such as a trimeric H-NOX protein by methods known in the art and by the non-limiting exemplary methods described below.

Kinetic K_{D}: Ratio of k_{off} to kₒₙ

The kinetic K_{D} value is determined for wild-type and mutant H-NOX proteins, including polymeric H-NOS proteins, essentially as described by Boon, E. M. et al. (2005). Nature Chemical Biology 1:53-59, which is hereby incorporated by reference in its entirety, particularly with respect to the measurement of O₂ association rates, O₂ dissociation rates, dissociation constants for O₂ binding, autoxidation rates, and NO dissociation rates.

kₒₙ (O₂ Association Rate)

O₂ association to the heme is measured using flash photolysis at 20° C. It is not possible to flash off the Fe^{II}-O₂ complex as a result of the very fast geminate recombination kinetics; thus, the Fe^{II}-CO complex is subjected to flash photolysis with laser light at 560 nm (Hewlett-Packard, Palo Alto, CA), producing the 5-coordinate Fell intermediate, to which the binding of molecular O₂ is followed at various wavelengths. Protein samples are made by anaerobic reduction with 10 mM dithionite, followed by desalting on a PD-10 column (Millipore, Inc., Billerica, MA). The samples are then diluted to 20 μM heme in 50 mM TEA, 50 mM NaCl, pH 7.5 buffer in a controlled-atmosphere quartz cuvette, with a size of 100 μL to 1 mL and a path-length of 1-cm. CO gas is flowed over the headspace of this cuvette for 10 minutes to form the Fell-CO complex, the formation of which is verified by UV-visible spectroscopy (Soret maximum 423 nm). This sample is then either used to measure CO-rebinding kinetics after flash photolysis while still under 1 atmosphere of CO gas, or it is opened and stirred in air for 30 minutes to fully oxygenate the buffer before flash photolysis to watch O₂-rebinding events. O₂ association to the heme is monitored at multiple wavelengths versus time. These traces are fit with a single exponential using Igor Pro software (Wavemetrics, Inc., Oswego, OR; latest 2005 version). This rate is independent of observation wavelength but dependent on O₂ concentration. UV-visible spectroscopy is used throughout to confirm all the complexes and intermediates (Cary 3K, Varian, Inc. Palo Alto, CA). Transient absorption data are collected using instruments described in Dmochowski, I. J. et al. (Aug. 31, 2000). J Inorg Biochem. 81 (3): 221-228, which is hereby incorporated by reference in its entirety, particularly with respect to instrumentation. The instrument has a response time of 20 ns, and the data are digitized at 200 megasamples s⁻¹.

k_{off} (O₂ dissociation rate)

To measure the k_{off}, Fe^{II}-O₂ complexes of protein (5 μM heme), are diluted in anaerobic 50 mM TEA, 50 mM NaCl, pH 7.5 buffer, and are rapidly mixed with an equal volume of the same buffer (anaerobic) containing various concentrations of dithionite and/or saturating CO gas. Data are acquired on a HI-TECH Scientific SF-61 stopped-flow spectrophotometer equipped with a Neslab RTE-100 constant-temperature bath set to 20° C. (TGK Scientific LTD., Bradford On Avon, United Kingdom). The dissociation of O₂ from the heme is monitored as an increase in the absorbance at 437 nm, a maximum in the Fell-Fell-02 difference spectrum, or 425 nm, a maximum in the Fell-Fell-CO difference spectrum. The final traces are fit to a single exponential using the software that is part of the instrument. Each experiment is done a minimum of six times, and the resulting rates are averaged. The dissociation rates measured are independent of dithionite concentration and independent of saturating CO as a trap for the reduced species, both with and without 10 mM dithionite present.

### Kinetic K_{D}

The kinetic Kpis determined by calculating the ratio of k_{off} to kₒₙ using the measurements of k_{off} and kₒₙ described above.

### Calculated K_{D}

To measure the calculated K_{D}), the values for the k_{off} and kinetic K_{D} that are obtained as described above are graphed. A linear relationship between k_{off} and kinetic K_{D} is defined by the equation (y=mx+b). k_{off} values were then interpolated along the line to derive the calculated K_{D} using Excel: MAC 2004 (Microsoft, Redmond, WA). In the absence of a measured kₒₙ, this interpolation provides a way to relate k_{off} to K_{D}.

### Rate of Autoxidation

To measure the rate of autoxidation, the protein samples are anaerobically reduced, then diluted to 5 μM heme in aerobic 50 mM TEA, 50 mM NaCl, pH 7.5 buffer. These samples are then incubated in a Cary 3E spectrophotometer equipped with a Neslab RTE-100 constant-temperature bath set to 37° C. and scanned periodically (Cary 3E, Varian, Inc., Palo Alto, CA). The rate of autoxidation is determined from the difference between the maximum and minimum in the Fe^{III}-Fe^{II} difference spectrum plotted versus time and fit with a single exponential using Excel: MAC 2004 (Microsoft, Redmond, WA).

Rate of Reaction with NO

NO reactivity is measured using purified proteins (H-NOX, polymeric H-NOX, Homo sapiens hemoglobin (Hs Hb) etc.) prepared at 2 μM in buffer A and NO prepared at 200 μM in Buffer A (Buffer A: 50 mM Hepes, pH 7.5, 50 mM NaCl). Data are acquired on a HI-TECH Scientific SF-61 stopped-flow spectrophotometer equipped with a Neslab RTE-100 constant-temperature bath set to 20° C. (TGK Scientific LTD., Bradford On Avon, United Kingdom). The protein is rapidly mixed with NO in a 1:1 ratio with an integration time of 0.00125 sec. The wavelengths of maximum change are fit to a single exponential using the software that is part of the spectrometer, essentially measuring the rate-limiting step of oxidation by NO. The end products of the reaction are ferric-NO for the HNOX proteins and ferric-aquo for Hs Hb.

p50 measurements

If desired, the p50 value for mutant or wild-type H-NOX proteins can be measured as described by Guarnone, R. et al. (September/October 1995). Haematologica 80 (5): 426-430, which is hereby incorporated by reference in its entirety, particularly with respect to the measurement of p50 values. The p50 value is determined using a HemOx analyzer. The measurement chamber starts at 0% oxygen and slowly is raised, incrementally, towards 100% oxygen. An oxygen probe in the chamber measures the oxygen saturation %. A second probe (UV-Vis light) measures two wavelengths of absorption, tuned to the alpha and beta peaks of the hemoprotein's (e.g., a protein such as H-NOX complexed with heme) UV-Vis spectra. These absorption peaks increase linearly as hemoprotein binds oxygen. The percent change from unbound to 100% bound is then plotted against the % oxygen values to generate a curve. The p50 is the point on the curve where 50% of the hemoprotein is bound to oxygen.

Specifically, the Hemox-Analyzer (TCS Scientific Corporation, New Hope, PA) determines the oxyhemoprotein dissociation curve (ODC) by exposing 50 μL of blood or hemoprotein to an increasing partial pressure of oxygen and deoxygenating it with nitrogen gas. A Clark oxygen electrode detects the change in oxygen tension, which is recorded on the x-axis of an x-y recorder. The resulting increase in oxyhemoprotein fraction is simultaneously monitored by dual-wavelength spectrophotometry at 560 nm and 576 nm and displayed on the y-axis. Blood samples are taken from the antemedial vein, anticoagulated with heparin, and kept at 4° C. on wet ice until the assay. Fifty μL of whole blood are diluted in 5 μL of Hemox-solution, a manufacturer-provided buffer that keeps the pH of the solution at a value of 7.4±0.01. The sample-buffer is drawn into a cuvette that is part of the Hemox-Analyzer and the temperature of the mixture is equilibrated and brought to 37° C.; the sample is then oxygenated to 100% with air. After adjustment of the pO₂ value the sample is deoxygenated with nitrogen; during the deoxygenation process the curve is recorded on graph paper. The P50 value is extrapolated on the x-axis as the point at which O₂ saturation is 50% using the software that is part of the Hemox-Analyzer. The time required for a complete recording is approximately 30 minutes.

### H-NOX Nucleic Acids

The invention also features nucleic acids encoding any of the mutant H-NOX proteins, polymeric H-NOX, or recombinant monomer H-NOX protein subunits as described herein.

In particular embodiments, the nucleic acid includes a segment of or the entire nucleic acid sequence of any of nucleic acids encoding an H-NOX protein or an H-NOX domain. In some embodiments, the nucleic acid includes at least about 50, 100, 150, 200, 300, 400, 500, 600, 700, 800, or more contiguous nucleotides from a H-NOX nucleic acid and contains one or more mutations (e.g., 1, 2, 3, 4, 5, 6, 7, 8, 9, or 10 mutations) compared to the H-NOX nucleic acid from which it was derived. In various embodiments, a mutant H-NOX nucleic acid contains less than about 20, 15, 12, 10, 9, 8, 7, 6, 5, 4, 3, or 2 mutations compared to the H-NOX nucleic acid from which it was derived. The invention also features degenerate variants of any nucleic acid encoding a mutant H-NOX protein.

In some embodiments, the nucleic acid includes nucleic acids encoding two or more H-NOX domains. In some embodiments, the nucleic acids including two or more H-NOX domains are linked such that a polymeric H-NOX protein is expressed from the nucleic acid. In further embodiments, the nucleic acid includes nucleic acids encoding one or more polymerization domains. In some embodiments, the nucleic acids including the two or more H-NOX domains and the one or more polymerization domains are linked such that a polymeric H-NOX protein is expressed from the nucleic acid.

In some embodiments, the nucleic acid includes a segment or the entire nucleic acid sequence of any nucleic acid encoding a polymerization domain. In some embodiments the nucleic acid comprises a nucleic acid encoding an H-NOX domain and a polymerization domain. In some embodiments, the nucleic acid encoding an H-NOX domain and the nucleic acid encoding a polymerization domain a linked such that the produced polypeptide is a fusion protein comprising an H-NOX domain and a polymerization domain.

In some embodiments, the nucleic acid comprises nucleic acid encoding one or more His₆ tags. In some embodiments the nucleic acid further comprised nucleic acids encoding linker sequences positioned between nucleic acids encoding the H-NOX domain, the polymerization domain and/or a His₆ tag.

In some embodiments, the invention provides a nucleic acid encoding an H-NOX domain and a foldon domain. In some embodiments, the H-NOX domain is a T. thermoanaerobacter H-NOX domain. In some embodiments, the H-NOX domain is a wild-type T. thermoanaerobacter H-NOX domain. In some embodiments, the H-NOX domain is a T. thermoanaerobacter L144F H-NOX domain. In some embodiments, the H-NOX domain is a T. thermoanaerobacter W9F/L144F H-NOX domain.

In some embodiments, the invention provides nucleic acids encoding the following 5′ to 3′: a L144F T. tengcongensis H-NOX domain, a Gly-Ser-Gly amino acid linker sequence, and a foldon domain. In some embodiments, the invention provides nucleic acids encoding the following 5′ to 3′: a W9F/L144F T. tengcongensis H-NOX domain, a Gly-Ser-Gly amino acid linker sequence, and a foldon domain. In some embodiments, the invention provides nucleic acids encoding the following 5′ to 3′: a wild-type T. tengcongensis H-NOX domain, a Gly-Ser-Gly amino acid linker sequence, and a foldon domain.

In some embodiments, the invention provides nucleic acids encoding the following 5′ to 3′: a L144F T. tengcongensis H-NOX domain, a Gly-Ser-Gly amino acid linker sequence, a foldon domain, an Arg-Gly-Ser linker sequence, and a His tag. In some embodiments, the invention provides nucleic acids encoding the following 5′ to 3′: a W9F/L144F T. tengcongensis H-NOX domain, a Gly-Ser-Gly amino acid linker sequence, a foldon domain, an Arg-Gly-Ser linker sequence, and a His₆ tag. In some embodiments, the invention provides nucleic acids encoding the following 5′ to 3′: a wild-type T. tengcongensis H-NOX domain, a Gly-Ser-Gly amino acid linker sequence, a foldon domain, an Arg-Gly-Ser linker sequence, and a His₆ tag.

In some embodiments, the nucleic acid comprises the nucleic acid sequence set forth in SEQ ID NO:5 or SEQ ID NO:7.

The invention also includes a cell or population of cells containing at least one nucleic acid encoding a mutant H-NOX protein described herein. Exemplary cells include insect, plant, yeast, bacterial, and mammalian cells. These cells are useful for the production of mutant H-NOX proteins using standard methods, such as those described herein.

In some embodiments, the invention provides a cell comprising a nucleic acid encoding an H-NOX domain and a foldon domain. In some embodiments, the H-NOX domain is a T. thermoanaerobacter H-NOX domain. In some embodiments, the H-NOX domain is a wild-type T. thermoanaerobacter H-NOX domain. In some embodiments, the H-NOX domain is a T. thermoanaerobacter L144F H-NOX domain. In some embodiments, the H-NOX domain is a T. thermoanaerobacter W9F/L144F H-NOX domain. In some embodiments, the invention provides a cell comprising a nucleic acid comprising the nucleic acid sequence set forth in SEQ ID NO: 5 or SEQ ID NO:7.

### Formulations of H-NOX Proteins

The present invention provides formulations of O₂ carrier polypeptides for use in enhancing tumor immunogenicity; for example, by inhibiting the immune suppressive activities associated with tumor hypoxia. A non-limiting exemplary family of O₂ carrier polypeptides is the H-NOX family of O₂ carrier polypeptides. Any wild-type or mutant H-NOX protein, including polymeric H-NOX proteins, described herein may be used for the formulation of pharmaceutical or non-pharmaceutical compositions. In some embodiments, the formulations comprise a monomeric H-NOX protein comprising an H-NOX domain and a polymerization domain such that the monomeric H-NOX proteins associate in vitro or in vivo to produce a polymeric H-NOX protein. As discussed further below, these formulations are useful in a variety of therapeutic and industrial applications.

In some embodiments, the pharmaceutical composition includes one or more wild-type or mutant H-NOX proteins described herein including polymeric H-NOX proteins and a pharmaceutically acceptable carrier or excipient. Examples of pharmaceutically acceptable carriers or excipients include, but are not limited to, any of the standard pharmaceutical carriers or excipients such as phosphate buffered saline solutions, water, emulsions such as oil/water emulsion, and various types of wetting agents. Exemplary diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000, which are each hereby incorporated by reference in their entireties, particularly with respect to formulations). In some embodiments, the formulations are sterile. In some embodiments, the formulations are essentially free of endotoxin.

While any suitable carrier known to those of ordinary skill in the art may be employed in the pharmaceutical compositions of this invention, the type of carrier will vary depending on the mode of administration. Compositions can be formulated for any appropriate manner of administration, including, for example, intravenous, intra-arterial, intravesicular, intratumoral, inhalation, intraperitoneal, intrapulmonary, intramuscular, subcutaneous, intra-tracheal, transmucosal, intraocular, intrathecal, or transdermal administration. For parenteral administration, such as subcutaneous injection, the carrier may include, e.g., water, saline, alcohol, a fat, a wax, or a buffer. For oral administration, any of the above carriers or a solid carrier, such as mannitol, lactose, starch, magnesium stearate, sodium saccharine, talcum, cellulose, glucose, sucrose, or magnesium carbonate, may be employed. Biodegradable microspheres (e.g., polylactate polyglycolate) may also be used as carriers.

In some embodiments, the pharmaceutical or non-pharmaceutical compositions include a buffer (e.g., neutral buffered saline, phosphate buffered saline, etc), a carbohydrate (e.g., glucose, mannose, sucrose, dextran, etc.), an antioxidant, a chelating agent (e.g., EDTA, glutathione, etc.), a preservative, another compound useful for binding and/or transporting oxygen, an inactive ingredient (e.g., a stabilizer, filler, etc.), or combinations of two or more of the foregoing. In some embodiments, the composition is formulated as a lyophilizate. H-NOX proteins may also be encapsulated within liposomes or nanoparticles using well known technology. Other exemplary formulations that can be used for H-NOX proteins are described by, e.g., U.S. Pat. Nos. 6,974,795, and 6,432,918, which are each hereby incorporated by reference in their entireties, particularly with respect to formulations of proteins.

The compositions described herein may be administered as part of a sustained release formulation (e.g., a formulation such as a capsule or sponge that produces a slow release of compound following administration). Such formulations may generally be prepared using well known technology and administered by, for example, oral, rectal or subcutaneous implantation, or by implantation at the desired target site. Sustained-release formulations may contain an H-NOX protein dispersed in a carrier matrix and/or contained within a reservoir surrounded by a rate controlling membrane. Carriers for use within such formulations are biocompatible, and may also be biodegradable. In some embodiments, the formulation provides a relatively constant level of H-NOX protein release. The amount of H-NOX protein contained within a sustained release formulation depends upon the site of implantation, the rate and expected duration of release, and the nature of the condition to be treated or prevented.

In some embodiments, the pharmaceutical composition contains an effective amount of a wild-type or mutant H-NOX protein. In some embodiments, the pharmaceutical composition contains an effective amount of a polymeric H-NOX protein comprising two or more wild-type or mutant H-NOX domains. In some embodiments, the pharmaceutical composition contains an effective amount of a recombinant monomeric H-NOX protein comprising a wild-type or mutant H-NOX domain and a polymerization domain as described herein. In some embodiments, the formulation comprises a trimeric H-NOX protein comprising three monomers, each monomer comprising a T. tengcongensis L144F H-NOX domain and a foldon domain. In some embodiments, the formulation comprises a trimeric H-NOX protein comprising three monomers, each monomer comprising a T. tengcongensis W9F/L144F H-NOX domain and a foldon domain. In some embodiments, the formulation comprises a trimeric H-NOX protein comprising three monomers, each monomer comprising a T. tengcongensis L144F H-NOX domain and a foldon domain. In some embodiments, the formulation comprises a PEGylated trimeric H-NOX protein comprising three monomers, each monomer comprising a T. tengcongensis L144F H-NOX domain and a foldon domain. In some embodiments, the pharmaceutical composition comprises an O₂ carrier polypeptide (e.g., an H-NOX protein) in an amount effective to modulate tumor immunity (e.g., enhance an immune response to the tumor).

In some embodiments, an effective amount of an H-NOX protein for administration to a human is between a few grams to over about 350 grams. Other exemplary doses of an H-NOX protein include about any of 4.4, 5, 10, or 13 G/DL (where G/DL is the concentration of the H-NOX protein solution prior to infusion into the circulation) at an appropriate infusion rate, such as about 0.5 ml/min (see, for example, Winslow, R. Chapter 12 In Blood Substitutes). It will be appreciated that the unit content of active ingredients contained in an individual dose of each dosage form need not in itself constitute an effective amount since the necessary effective amount could be reached by the combined effect of a plurality of administrations. The selection of the amount of an H-NOX protein to include in a pharmaceutical composition depends upon the dosage form utilized, the condition being treated, and the particular purpose to be achieved according to the determination of the ordinarily skilled artisan in the field.

Exemplary compositions include genetically engineered, recombinant H-NOX proteins, which may be isolated or purified, comprising one or more mutations that collectively impart altered O₂ or NO ligand-binding relative to the corresponding wild-type H-NOX protein, and operative as a physiologically compatible mammalian blood gas carrier. For example, mutant H-NOX proteins as described herein. In some embodiments, the H-NOX protein is a polymeric H-NOX protein. In some embodiments, the H-NOX protein is a recombinant monomeric H-NOX protein comprising a wild-type or mutant H-NOX domain and a polymerization domain as described herein. In some embodiments, the composition comprises a trimeric H-NOX protein comprising three monomers, each monomer comprising a T. tengcongensis L144F H-NOX domain and a foldon domain. In some embodiments, the composition comprises a trimeric H-NOX protein comprising three monomers, each monomer comprising a T. tengcongensis W9F/L144F H-NOX domain and a foldon domain. In some embodiments, the composition comprises a trimeric H-NOX protein comprising three monomers, each monomer comprising a T. tengcongensis L144F H-NOX domain and a foldon domain. In some embodiments, the composition comprises a PEGylated trimeric H-NOX protein comprising three monomers, each monomer comprising a T. tengcongensis L144F H-NOX domain and a foldon domain.

To reduce or prevent an immune response in human subjects who are administered a pharmaceutical composition, human H-NOX proteins or domains (either wild-type human proteins or human proteins into which one or more mutations have been introduced) or other non-antigenic H-NOX proteins or domains (e.g., mammalian H-NOX proteins) can be used. To reduce or eliminate the immunogenicity of H-NOX proteins derived from sources other than humans, amino acids in an H-NOX protein or H-NOX domain can be mutated to the corresponding amino acids in a human H-NOX. For example, one or more amino acids on the surface of the tertiary structure of a non-human H-NOX protein can be mutated to the corresponding amino acid in a human H-NOX protein.

### Methods to Modulate Tumor Immunity

In some aspects, the invention provides methods modulate to tumor immunity and thus can be used in anticancer treatments. Hypoxic tumor microenvironments suppress the host's immune anti-tumor defenses by modulating multiple signaling pathways (FIG. 1) including, but not limited to, hypoxia inducible factor (HIF-1) signaling (Codo et al., 2014 Oncotarget, 5 (17), 7651-7662; Lee, Mace, & Repasky, 2010 Int J Hyperthermia, 26 (3), 232-246; Wei et al., 2011 PLoS One, 6 (1), e16195), miRNA epigenetic regulation of antitumor T cells, MHC1 expression on tumor cells, and recruitment of tumor associated macrophages and myeloid-derived suppressor cells (MDSC). Hypoxic activation of the HIF-1 pathway has been shown to activate adenosinergic A2 and PD-L1 pathways which in turn inhibit recruitment and activation of helper and killer T-cells and NK cells (Noman et al., 2014 J Exp Med, 211 (5), 781-790; Ohta et al., 2006 Proc Natl Acad Sci USA, 103 (35), 13132-13137). Hypoxic activation of the HIF-1 pathway may also lead to the recruitment and activation of inhibitory regulatory T cells (Treg), tumor associated macrophages (TAM) and other myeloid-derived suppressor cells (MDSC) (Chaturvedi et al., 2014 Proc Natl Acad Sci USA, 111 (20), E2120-2129; Corzo et al., 2010 J Exp Med, 207 (11), 2439-2453; Wei et al., 2011). HIF-1 pathway activation may also directly inhibit the ability of tumor cells to be recognized by immune system by increasing tumor shedding of MHC1 receptors (Siemens et al., 2008 Cancer Res, 68 (12), 4746-4753).

In some aspects, the invention provides methods for modulating tumor immunity in an individual with a tumor comprising administering to the individual an effective amount of an O₂ carrier polypeptide (e.g., an H-NOX protein). In some embodiments, the modulating of tumor immunity comprises enhancing an immune response to the tumor. In some embodiments, the invention provides methods for increasing leucocyte infiltration to a tumor in an individual comprising administering to the individual an effective amount of an O₂ carrier polypeptide. In some embodiments, the invention provides methods for increasing lymphocyte infiltration to a tumor in an individual comprising administering to the individual an effective amount of an O₂ carrier polypeptide. In some embodiments, the increase in lymphocyte infiltration to the tumor comprises an increase in infiltration of one or more of CD4 cells, CD8 cells, or NK cells. In some embodiments, the modulating of tumor immunity comprises increasing antigen processing. In some embodiments, the modulating of tumor immunity comprises increasing the presentation capabilities of dendritic cells (DC). In some embodiments, the modulating of tumor immunity comprises one or more of increasing lymphocyte infiltration to the tumor, increasing antigen processing, or increasing DC presentation capability. In some embodiments, the modulating of tumor immunity comprises lymphocyte activation. In some embodiments, the modulateing of tumor immunity comprises cytokine secretion. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments of the invention, the increase in lymphocyte infiltration to the tumor is accompanied by inhibition of one or more of Treg cells, tumor associated macrophages or myeloid derived suppressor cells in the tumor. In some embodiments, the increase in lymphocyte infiltration to the tumor is accompanied by an increase in MHC1 expression on the tumor cells.

In some embodiments, the invention provides methods for decreasing expression of HIF-1α in a tumor in an individual comprising administering to the individual an effective amount of an O₂ carrier polypeptide (e.g. an H-NOX protein). In some embodiments, administration of an effective amount of an O₂ carrier polypeptide (e.g., an H-NOX protein) to an individual results in a decrease in expression of HIF-1α. In some embodiments, the expression of HIF-1α is decreased by more than about any of 10%, 20%, 30%, 40%, 50%, 60%, 70%, 80%, 90% or 100% compared to expression of HIF-1α in the absence of treatment with an O₂ carrier polypeptide. In some embodiments, the expression of HIF-1α is reduced compared to expression of HIF-1α in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for decreasing expression of HIF-1α in a tumor in an individual comprising administering to the individual an effective amount of an O₂ carrier polypeptide (e.g. an H-NOX protein) wherein the decrease in expression of HIF-1α is measured as a decrease in expression of vascular epithelial cell growth factor (VEGF). In some embodiments, administration of an effective amount of an O₂ carrier polypeptide (e.g., an H-NOX protein) to an individual results in a decrease in expression of VEGF. In some embodiments, the expression of VEGF is decreased by more than about any of 10%, 20%, 30%, 40%, 50%, 60%, 70%, 80%, 90% or 100% compared to expression of VEGF in the absence of treatment with an O₂ carrier polypeptide. In some embodiments, the expression of VEGF is reduced compared to expression of VEGF in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for decreasing expression of HIF-1α in a tumor in an individual comprising administering to the individual an effective amount of an O₂ carrier polypeptide (e.g. an H-NOX protein) herein the decrease in expression of HIF-1α is measured as a decrease in expression of glucose transporter type 1 (Glut1). In some embodiments, administration of an effective amount of an O₂ carrier polypeptide (e.g., an H-NOX protein) to an individual results in a decrease in expression of Glut1. In some embodiments, the expression of Glut1 is decreased by more than about any of 10%, 20%, 30%, 40%, 50%, 60%, 70%, 80%, 90% or 100% compared to expression of Glut1 in the absence of treatment with an O₂ carrier polypeptide. In some embodiments, the expression of Glut1 is reduced compared to expression of Glut1 in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for decreasing expression of PD-L1 in a tumor in an individual comprising administering to the individual an effective amount of an O₂ carrier polypeptide (e.g. an H-NOX protein). In some embodiments, administration of an effective amount of an O₂ carrier polypeptide (e.g., an H-NOX protein) to an individual results in a decrease in expression of PD-L1. In some embodiments, the expression of PD-L1 is decreased by more than about any of 10%, 20%, 30%, 40%, 50%, 60%, 70%, 80%, 90% or 100% compared to expression of PD-L1 in the absence of treatment with an O₂ carrier polypeptide. In some embodiments, administration of an effective amount of an O₂ carrier polypeptide (e.g., an H-NOX protein) to an individual results in a decrease in the interaction of PD-L1 with PD-1. In some embodiments, the interaction of PD-L1 with PD1 is decreased by more than about any of 10%, 20%, 30%, 40%, 50%, 60%, 70%, 80%, 90% or 100% compared to interaction of PD-L1 with PD1 in the absence of treatment with an O₂ carrier polypeptide. In some embodiments, the expression of PD-L1 is reduced compared to expression of PD-L1 in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for decreasing expression of A2AR in a tumor in an individual comprising administering to the individual an effective amount of an O₂ carrier polypeptide (e.g. an H-NOX protein). In some embodiments, administration of an effective amount of an O₂ carrier polypeptide (e.g., an H-NOX protein) to an individual results in a decrease in expression of A2AR. In some embodiments, the expression of A2AR is decreased by more than about any of 10%, 20%, 30%, 40%, 50%, 60%, 70%, 80%, 90% or 100% compared to expression of A2AR in the absence of treatment with an O₂ carrier polypeptide. In some embodiments, administration of an effective amount of an O₂ carrier polypeptide (e.g., an H-NOX protein) to an individual results in a decrease in the interaction of A2AR with adenosine. In some embodiments, the interaction of A2AR with adenosine is decreased by more than about any of 10%, 20%, 30%, 40%, 50%, 60%, 70%, 80%, 90% or 100% compared to interaction of A2AR with adenosine in the absence of treatment with an O₂ carrier polypeptide. In some embodiments, the expression of A2AR is reduced compared to expression of A2AR in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for decreasing expression of HIF-2a in a tumor in an individual comprising administering to the individual an effective amount of an O₂ carrier polypeptide (e.g. an H-NOX protein). In some embodiments, administration of an effective amount of an O₂ carrier polypeptide (e.g., an H-NOX protein) to an individual results in a decrease in expression of HIF-2a. In some embodiments, the expression of HIF-2a is decreased by more than about any of 10%, 20%, 30%, 40%, 50%, 60%, 70%, 80%, 90% or 100% compared to expression of HIF-2a in the absence of treatment with an O₂ carrier polypeptide. In some embodiments, administration of an effective amount of an O₂ carrier polypeptide (e.g., an H-NOX protein) to an individual results in a decrease in the expression of HIF-2α. In some embodiments, the expression of HIF-2α is decreased by more than about any of 10%, 20%, 30%, 40%, 50%, 60%, 70%, 80%, 90% or 100% compared to expression of HIF-2α in the absence of treatment with an O₂ carrier polypeptide. In some embodiments, the expression of HIF-2a is reduced compared to expression of HIF-2a in the absence of treatment with an O₂ carrier protein for more than about any of 1 hr, 2 hr, 3 hr, 4 hr, 5 hr, 6 hr, 8 hr, 10 hr, 12 hr, 16 hr, 20 hr, 24 hr, 30 hr, 36, hr 42 hr or 48 hr. In some embodiments, the O₂ carrier polypeptide is a trimeric Tt H-NOX L144F polypeptide. In some embodiments, the O₂ carrier polypeptide is a PEGylated trimeric Tt H-NOX L144F polypeptide.

In some embodiments, the invention provides methods for modulating tumor immunity (e.g., enhancing an immune response to a tumor) in an individual by any of the methods described herein. Examples of tumors include but are not limited to a brain tumor, a glioblastoma, a bone tumor, a pancreatic tumor, a skin tumor, a tumor of the head or neck, a melanoma, a lung tumor, a uterine tumor, an ovarian tumor, a colorectal tumor, an anal tumor, a liver tumor, a hepatocellular carcinoma, a stomach tumor, a testicular tumor, an endometrial tumor, a cervical tumor, a vaginal tumor, a Hodgkin's lymphoma, a non-Hodgkin's lymphoma, an esophageal tumor, an intestinal tumor, a thyroid tumor, an adrenal tumor, a bladder tumor, a kidney tumor, a breast tumor, a multiple myeloma tumor, a sarcoma, or a squamous cell tumor.

In some embodiments, the invention provides methods for modulating tumor immunity (e.g., enhancing an immune response to a tumor) in an individual by any of the methods described herein thereby providing methods for treating cancer in an individual. Examples of cancers that may be treated by the methods of the invention include but are not limited to brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma, or squamous cell cancer.

In some embodiments, the invention provides methods for modulating tumor immunity in an individual by any of the methods described herein. In some embodiments, the individual is a mammal; for example a human. In some embodiments, the mammal is a pet, a laboratory research animal, or a farm animal. Non-limiting examples of pets, research animals or farm animals include dogs, cats, horses, monkeys, rabbits, rats, mice, guinea pigs, hamsters, pigs and cows.

O₂ carrier polypeptides may be administered by any route including but not limited to intravenous, intra-arterial, intratumoral, intravesicular, inhalation, intraperitoneal, intrapulmonary, intramuscular, subcutaneous, intra-tracheal, transmucosal, intraocular, intrathecal, or transdermal administration.

In some aspects, sustained delivery of oxygen to a tumor is desired to inhibit hypoxia-mediated tumor immunity and to enhance an immune response to the tumor. In some embodiments of the invention, administration of the O₂ carrier polypeptide (e.g., H-NOX protein) is repeated. Administration of the O₂ carrier polypeptide may be repeated until a robust immune response to the tumor is established. In some embodiments, administration of the O₂ carrier polypeptide is repeated at least about any one of two times, three times, four times, five times, six times, seven times, eight times, nine times, ten times, twelve times, fourteen times, twenty times, thirty times, forty times, fifty times or one hundred times. In some embodiments, administration of the O₂ carrier polypeptide is repeated between about two times and about twenty times. In some embodiments, administration of the O₂ carrier polypeptide is repeated between any one of about twenty times and about forty times, any one of about forty times and about sixty times, any one of about sixty times and about eighty times, any one of about eighty times and about one hundred times, or any number of times therebetween. In some embodiments, administration of the O₂ carrier polypeptide is repeated daily or twice a day for about 42 to about 84 administrations.

Exemplary dosing frequencies include, but are not limited to, at least 1, 2, 3, 4, 5, 6, or 7 times (i.e., daily) a week. In some embodiments, the O₂ carrier polypeptide (e.g., H-NOX protein) is administered at least 2, 3, 4, or 6 times a day. In some embodiments, the O₂ carrier polypeptide is administered every four, every 8, every 12, every 24 hours, every 48 hours or two times a week or three times a week. In some embodiments, the O₂ carrier polypeptide is administered any one of between one hour and two hours, between two hours and four hours, between four hours and eight hours, between eight hours and twelve hours, or between twelve hours and 24 hours. In some embodiments, the O₂ carrier polypeptide is administered every four, every 8, every 12 or every 24 hours for a period of about one to about 10 days. In some embodiments, the O₂ carrier polypeptide can be administered, e.g., over a period of a few days or weeks. In some embodiments, the O₂ carrier polypeptide is administrated for a longer period, such as a few months or years. The dosing frequency of the composition may be adjusted over the course of the treatment based on the judgment of the administering physician.

In some embodiments, the O₂ carrier polypeptide (e.g., H-NOX protein) is administered as a bolus. In some embodiments, the volume of the bolus is greater than about any of 1 mL, 2 mL, 3 mL, 4 mL, 5 mL, 6 mL, 7 mL, 8 mL, 9 mL, 10 mL, 15 mL, 20 mL, 25 mL, 50 mL, 75 mL, or 100 mL. In some embodiments, administration of the bolus dose is repeated as above.

In some embodiments, the O₂ carrier polypeptide (e.g., H-NOX protein) is administered by infusion. In some embodiments, the infusions is for greater than about any of 15 minutes, 30 minutes, 1 hour, 2 hours, 3 hours, 4 hours, 5 hours, 6 hours, 7 hours, 8 hours, 9 hours, 10 hours, 12 hours, 16 hours, 20 hours or 24 hours. In some embodiments, the infusions is for between about any of 15 minutes and 30 minutes, 30 minutes and 1 hour, 1 hour and 2 hours, 2 hours and 3 hours, 3 hours and 4 hours, 4 hours and 5 hours, 5 hours and 6 hours, 6 hours and 7 hours, 7 hours and 8 hours, 8 hours and 9 hours, 9 hours and 10 hours, 10 hours and 12 hours, 12 hours and 16 hours, 16 hours and 20 hours or 20 hours and 24 hours. In some embodiments, the infusion rate is greater any of about 1 mL/hr, 2 mL/hr, 3 mL/hr, 4 mL/hr, 5 mL/hr, 6 mL/hr, 7 mL/hr, 8 mL/hr, 9 mL/hr, 10 mL/hr, 20 mL/hr, 30 mL/hr, 40 mL/hr, 50 mL/hr, 60 mL/hr, 70 mL/hr, 80 mL/hr, 90 mL/hr, 100 mL/hr, 200 mL/hr, 300 mL/hr, 400 mL/hr, 500 mL/hr, 600 mL/hr, 700 mL/hr, 800 mL/hr, 900 mL/hr, 1000 mL/hr, 2000 mL/hr, 3000 mL/hr, 4000 mL/hr, 5000 mL/hr, 6000 mL/hr, 7000 mL/hr, 8000 mL/hr, 9000 mL/hr, 10,000 mL/hr or any rate therebetween. In some embodiments, the infusion is repeated as above.

In some embodiments, the O₂ carrier polypeptide (e.g., H-NOX protein) is administered at a dose of greater than about any of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, or any dose therebetween. In some embodiments, the dose is provided as one or more bolus administrations. In some embodiments, the dose is provided as one or more infusions. In some embodiments the dose is provided in more than one administration (e.g., a dose of 100 mg/kg may be provided by two doses of 50 mg/kg).

In some embodiments of the invention, the O₂ carrier polypeptide (e.g. an H-NOX protein) is used in combination with radiation therapy. In some embodiments, the O₂ carrier polypeptide is administered to the individual any of at least 1, 2, 3, 4, 5, 6, 8, 10, 12, 14, 16, 18, 20, 22, or 24 hours before administration of the radiation. In some embodiments, the radiation is X irradiation. In some embodiments, the dose of X irradiation is any of about 0.5 Gy to about 75 Gy. In some embodiments, the cycle of O₂ carrier polypeptide administration and radiation administration is repeated any one of one, two, three, four, five or six times. In some embodiments, the cycle of O₂ carrier polypeptide administration and radiation administration is repeated after any one of about one week, two weeks, three weeks, four weeks, five weeks or six weeks. In some embodiments, the administration of the O₂ carrier polypeptide and radiation therapy is used in conjunction with another therapy; for example, a chemotherapy and/or immunotherapy.

In some embodiments of the invention, the O₂ carrier polypeptide (e.g. an H-NOX protein) is used in combination with chemotherapy. In some embodiments, the chemotherapy is a cytotoxin. Chemotherapeutic agents including cytotoxins are known in the art. In some embodiments, the cytotoxin is an alkylating agent. In some embodiments, the cytotoxin is cyclophosphamide or temozolomide. In some embodiments, the O₂ carrier polypeptide is administered before administration of the chemotherapy. In some embodiments, the O₂ carrier polypeptide is administered with administration of the chemotherapy. In some embodiments, the O₂ carrier polypeptide is administered after administration of the chemotherapy. In some embodiments, the O₂ carrier polypeptide is administered to the individual any of at least 1, 2, 3, 4, 5, 6, 7, 8, 10, 12, 14, 16, 18, 20, 22, or 24 hours or is administered daily or twice a day for 1, 2, 3, 4, 5, 6, or 7 days before administration of the chemotherapy. In some embodiments, the O₂ carrier polypeptide is administered to the individual any of at least 1, 2, 3, 4, 5, 6, 7, 8, 10, 12, or 24 hours after administration of the chemotherapy. In some embodiments, administration of the O₂ carrier polypeptide and/or administration of the chemotherapy is repeated any one of one, two, three, four, five, six, seven, eight, nine, ten times or more than ten times. In some embodiments, administration of the O₂ carrier polypeptide and/or administration of the chemotherapy is repeated after any one of about one week, two weeks, three weeks, four weeks, five weeks or six weeks. In some embodiments, administration of the O₂ carrier polypeptide and the chemotherapy are on the same dosing cycle. In some embodiments, administration of the O₂ carrier polypeptide and the chemotherapy are on different dosing cycles. In some embodiments, the admiration of H-NOX and radiation therapy is used in conjunction with another therapy; for example, radiation therapy and/or immunotherapy.

In some embodiments, the O₂ carrier polypeptide (e.g., an H-NOX protein) is administered to cancer patients prior to and/or in conjunction with an immunotherapy. In some embodiments, the immunotherapy is one or more of an anticancer vaccine, an adoptive immune cell therapy, an agent that targets an immune checkpoint regulator, an oncolytic virus or a BiTE. In some embodiments, the immunotherapy targets are one or more of CTLA-4, PD1, PD-L1, or an immune checkpoint regulator. In some embodiments, the immunotherapy is a dual PD1/CTLA-4 blockade therapy. In some embodiments, the immunotherapy is a PDL-1 treatment for patients with PDL1+ tumors or dual PD1/PD-L1 blockade. Nonlimiting examples include but are not limited to PD-1 and PDL-1 antagonists such as antibodies (e.g., Nivolumab). In some embodiments the checkpoint inhibitor is a CTLA4 antagonist such as an antibody (e.g., ipilumumab). In some embodiments, the immunotherapy is an adoptive T cell therapy including but not limited to chimeric antigen receptor T cells (e.g., CAR-T cells) or engineered TCR-T cells. In some embodiments, the immunotherapy is a Bispecific T cell Engagers (BiTE). In some embodiments, the immunotherapy includes one or more of anti-lymphocyte activation gene3 (LAG-3) therapy, anti-T cell immunoglobin mucin-3 (TIM-3) therapy, anti-killer immunoglobin-like receptos (KIR) thereapy, anti-4-1BB (CD137) agonizing/stimulatory therapy, or glucocorticoid-induced TNFR family related gene (GITR) agonizing/stimulatory therapy—each alone or in combinations with each other, and/or in combination with one or more of PD1, PDL-1, CTLA-4 or other therapies.

Nonlimiting examples of therapies that target checkpoint proteins other than PD-1/PDL-1 and CTLA4 negative regulators include both positive and negative (checkpoint inhibitors) regulators of immune response and can be antibodies or small molecules such as IDO (indoleamine-2.3 dioxygenase) pathway inhibitors such as direct IDO enzymatic activity inhibitors (e.g. NLG919), IDO effector pathway inhibitors (e.g. D-1-methyl-tryptophan, Indoximod, NLG8189), TDO (tryptophan 2,3-dioxygenase) inhibitors, or IDO-TDO dual inhibitors; Lymphocyte-activation gene 3 (LAG-3, CD223) antibody antagonists (e.g. IMP321, BMS-986016); Killer immunoglobulin-like receptors (KIRs) antagonists such as antibodies (e.g. lirilumab, IPH2101); T cell immunoglobulin mucin-3 (TIM-3) antagonists such as antibodies; B- and T cell attenuator (BTLA, CD272) antagonists such as antibodies; OX40 (CD134) agonists such as activating/stimulating antibodies; 4-1BB (CD137) agonists such as stimulatory antibodies (e.g. BMS-663513); Glucocorticoid-induced TNFR family related gene (GITR) agonists such as stimulatory antibodies (e.g. TRX518); and oncolytic viruses.

In some embodiments, the O₂ carrier polypeptide is administered before administration of the immunotherapy. In some embodiments, the O₂ carrier polypeptide is administered with administration of the immunotherapy. In some embodiments, the O₂ carrier polypeptide is administered after administration of the immunotherapy. In some embodiments, the O₂ carrier polypeptide is administered to the individual any of at least 1, 2, 3, 4, 5, 6, 7, 8, 10, 12, 24, or 48 hours before administration of the immunotherapy. In some embodiments, the O₂ carrier polypeptide is administered to the individual any of at least 3, 4, 5, 6, 7 or more days before administration of the immunotherapy. In some embodiments, the O₂ carrier polypeptide is administered to the individual any of at least 1, 2, 3, 4, 5, 6, 7, 8, 10, 12, 24 or 48 hours after administration of the immunotherapy. In some embodiments, the O₂ carrier polypeptide is administered to the individual any of at least 3, 4, 5, 6, 7 or more days after administration of the immunotherapy. In some embodiments, administration of the O₂ carrier polypeptide and/or administration of the immunotherapy is repeated any one of one, two, three, four, five, six, seven, eight, nine, ten times or more than ten times. In some embodiments, administration of the O₂ carrier polypeptide and/or administration of the immunotherapy is repeated after any one of about one week, two weeks, three weeks, four weeks, five weeks or six weeks. In some embodiments, administration of the O₂ carrier polypeptide and the immunotherapy are on the same dosing cycle. In some embodiments, administration of the O₂ carrier polypeptide and the immunotherapy are on different dosing cycles. In some embodiments, the admiration of H-NOX and radiation therapy is used in conjunction with another therapy; for example, radiation therapy and/or chemotherapy.

In some embodiments, the effectiveness of administration of the O₂ carrier polypeptide (e.g., H-NOX protein) is monitored; for example but not limited to tumor hypoxia, expression hypoxia-associated tumor suppressors and/or activators, presence of tumor-associated immune cells and/or immune cells directed against tumor cells and/or local (tumor biopsy, lymph node biopsy) or systemic (e.g. peripheral blood) cytokine and immune cell profiles. Methods to determine the level of tumor hypoxia are known in the art. Examples include but are not limited to measurement of any one of 18F-fluoromisonidazole (FMISO) tumor uptake, pimidazole uptake, 18F-fluoroazomycin arabinoside (FAZA) uptake, a nitroimidazole uptake, Copper (II)-diacetyl-bis (N4-methylthiosemicarbazone (Cu-ATSM) uptake, hexafluorobenzene (C6F6) uptake by 19F magnetic resonance imaging, hexamethyldisiloxane uptake by 1H MRI, tumor HIF-1α expression, tumor HIF-2a expression, tumor HIF-3a expression, tumor Glut-1 expression, tumor pH (pH-weighted MRI) qBOLD, OE-MRI, MOBILE MRI tumor LDHA expression, tumor carbonic anhydrase IX (CA-9) expression, VEGF expression, or lactate and/or pyruvate levels. In some embodiments of the methods of monitoring, treating, and optimization of therapy described above, tumor hypoxia is measured by 18F-FMISO uptake. In some embodiments, 18F-FMISO uptake is measured by Positron emission tomography (PET) scan, computed tomography (CT) scan or computed axial tomography (CAT) scan. Methods to detect expression of genes such as HIF-1α, PD-L1 and A2AR are known in the art; for example, by immunoassay, by immunohistochemistry, by quantitative PCR, by hybridization (for example, on a gene chip), and the like.

Kits with H-NOX Proteins

Also provided are articles of manufacture and kits for the modulation of tumor immunity in an individual. In some embodiments, the article of manufacture or kit comprises any of the O₂ carrier polypeptides including any of the H-NOX proteins described herein including polymeric H-NOX proteins and PEGylated polymeric H-NOX proteins, and suitable packaging. In some embodiments, the invention includes a kit with (i) a H-NOX protein (such as a wild-type or mutant H-NOX protein described herein or formulations thereof as described herein) and (ii) instructions for using the kit to deliver O₂ to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of H-NOX proteins generally include information regarding dosage, dosing schedule, and route of administration for the intended treatment or industrial use. The kit may further comprise a description of selecting an individual suitable for treatment.

The containers may be unit doses, bulk packages (e.g., multi-dose packages) or subunit doses. For example, kits may also be provided that contain sufficient dosages of H-NOX proteins disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of H-NOX proteins and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of H-NOX protein.

### Exemplary Methods for Production of H-NOX Proteins

As noted above, the sequences of several wild-type H-NOX proteins and nucleic acids are known and can be used to generate mutant H-NOX domains and nucleic acids of the present invention. Techniques for the mutation, expression, and purification of recombinant H-NOX proteins have been described by, e.g., Boon, E. M. et al. (2005). Nature Chemical Biology 1:53-59 and Karow, D. S. et al. (Aug. 10, 2004). Biochemistry 43 (31): 10203-10211, U.S. Pat. Nos. 8,404,631 and 8,404,632, WO 2007/139791, and WO 2007/139767 which are hereby incorporated by reference in their entireties, particularly with respect to the mutation, expression, and purification of recombinant H-NOX proteins. These techniques or other standard techniques can be used to generate any mutant H-NOX protein.

A mutant H-NOX nucleic acid can be incorporated into a vector, such as an expression vector, using standard techniques. For example, restriction enzymes can be used to cleave the mutant H-NOX nucleic acid and the vector. Then, the compatible ends of the cleaved mutant H-NOX nucleic acid and the cleaved vector can be ligated. The resulting vector can be inserted into a cell (e.g., an insect cell, a plant cell, a yeast cell, or a bacterial cell) using standard techniques (e.g., electroporation) for expression of the encoded H-NOX protein.

In particular, heterologous proteins have been expressed in a number of biological expression systems, such as insect cells, plant cells, yeast cells, and bacterial cells. Thus, any suitable biological protein expression system can be utilized to produce large quantities of recombinant H-NOX protein. In some embodiments, the H-NOX protein (e.g., a mutant or wild-type H-NOX protein) is an isolated protein.

If desired, H-NOX proteins can be purified using standard techniques. In some embodiments, the protein is at least about 60%, by weight, free from other components that are present when the protein is produced. In various embodiments, the protein is at least about 75%, 90%, or 99%, by weight, pure. A purified protein can be obtained, for example, by purification (e.g., extraction) from a natural source, a recombinant expression system, or a reaction mixture for chemical synthesis. Exemplary methods of purification include immunoprecipitation, column chromatography such as immunoaffinity chromatography, magnetic bead immunoaffinity purification, and panning with a plate-bound antibody, as well as other techniques known to the skilled artisan. Purity can be assayed by any appropriate method, e.g., by column chromatography, polyacrylamide gel electrophoresis, or HPLC analysis. In some embodiments, the purified protein is incorporated into a pharmaceutical composition of the invention or used in a method of the invention. The pharmaceutical composition of the invention may have additives, carriers, or other components in addition to the purified protein.

In some embodiments, the polymeric H-NOX protein comprises one or more His₆ tags. An H-NOX protein comprising at least one His₆ tag may be purified using chromatography; for example, using Ni²⁺-affinity chromatography. Following purification, the His₆ tag may be removed; for example, by using an exopeptidase. In some embodiments, the invention provides a purified polymeric H-NOX protein, wherein the polymeric H-NOX protein was purified through the use of a His₆ tag. In some embodiments, the purified H-NOX protein is treated with an exopeptidase to remove the His₆ tags.

In some embodiments, H-NOX protein comprises one or more molecules of polyethylene glycol (i.e., PEGylated). Methods to produce PEGylated proteins are known in the art.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Unless indicated otherwise, temperature is in degrees Centigrade and pressure is at or near atmospheric.

### Example 1. H-NOX Enables Efficient Oxygenation of Hypoxic Tumor Microenvironments

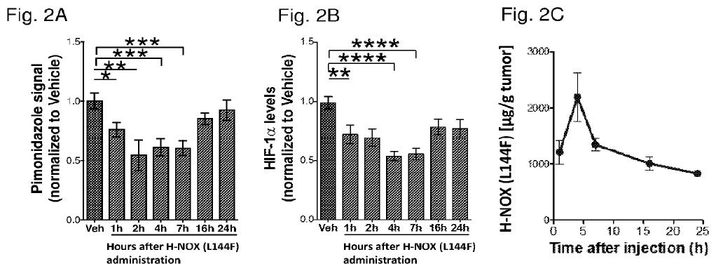

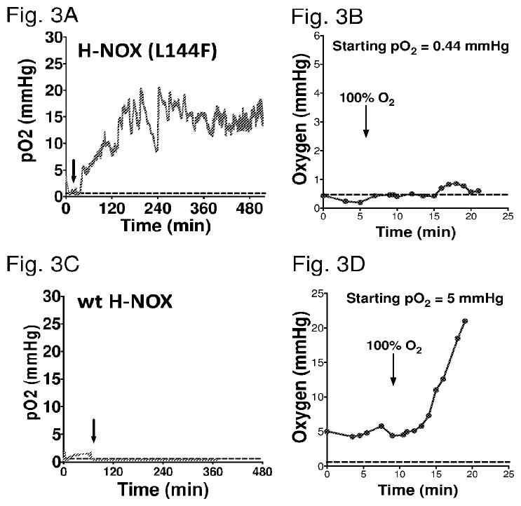

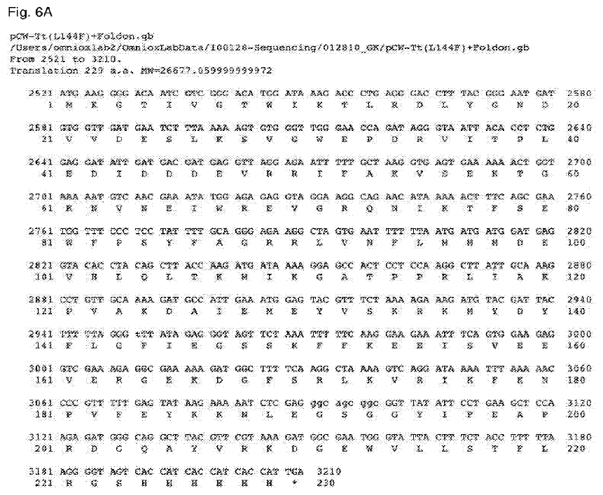

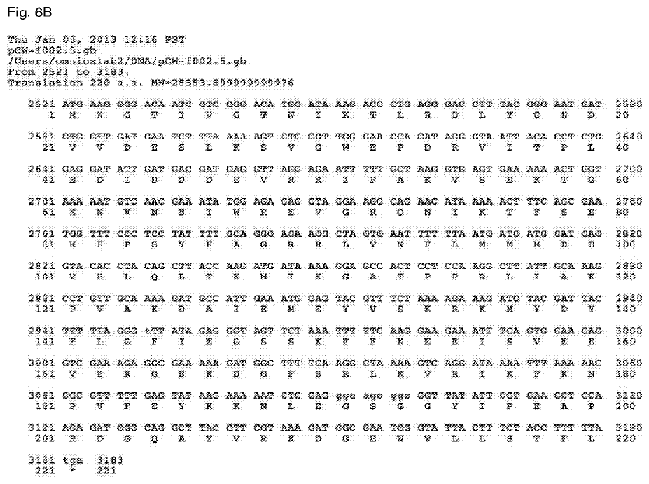

A PEGylated trimeric Thermoanaerobacter tengcongensis (Tt.) H-NOX bearing a L144F substitution in the distal pocket (FIG. 6B) was evaluated for the ability to oxygenate tumor microenvironments and increase radiation sensitivity. Administration of the PEGylated trimeric Tt H-NOX L144F to mice bearing hypoxic tumors induces rapid and sustained oxygenation of the tumors as directly measured by the external hypoxia marker, pimonidazole, hypoxia inducible transcription factor 1 alpha, HIF-1-α, and OxyLite oxygen-sensing nanofiber (FIGS. 2 and 3, respectively).

Mice bearing H460 subcutaneous xenograft tumors were injected i.v. with PEGylated trimer H-NOX (L144F) at 650 mg/kg when tumor volume reached ˜300-350 mm³ (˜10-14 days after tumor cell subcutaneous implantation). Prior to euthanasia, mice were injected with the exogenous hypoxia marker pimonidazole at 60 mg/kg and tumors were harvested. Pimonidazole (FIG. 2A) (Hypoxyprobe-1) and HIF-1α (FIG. 2B) levels were measured by competitive (pimonidazole) and sandwich (HIF-1α, Abcam) ELISAs, respectively. Graphs show quantification of pimonidazole and HIF-1α signals after PEGylated H-NOX (L144F) administration. Vehicle, 1 h and 4 h: n=22, 7 h: n=18, 12 h: n=16, 24 h: n=6. Results from 4 independent experiments. Mean values+/−SEM. ****p<0.0001, ***p<0.001, **p<0.01, * p<0.05 by one-way ANOVA and Bonferroni's post-hoc tests. (FIG. 2C) Tumors were assessed for the accumulation of PEGylated H-NOX (L144F) by sandwich H-NOX ELISA at 1, 4, 7, 16 and 24 hours after injection and results expressed per gram of tumor tissue. Seven to eight week old Nu/Nu female mice were subcutaneously implanted with 3×10⁶ of H460 human lung cancer cells and monitored until the tumors reached average size of ˜300 mm³ (10-14 days post-implantation of tumor cells). Mice bearing 200-350 mm³ xenograft tumors were injected i.v. with bolus vehicle (formulation buffer: 50 mM succinate, 50 mM NaCl, 3.4 mM EDTA, and 10 mM reduced glutathione at pH 7) or formulation buffer containing 650 mg/kg of PEGylated trimer H-NOX (L144F). To measure tumor hypoxia, prior to euthanasia, mice were injected with the exogenous hypoxia marker pimonidazole at 60 mg/kg. Tumors were harvested and homogenized in an extraction buffer (Abcam kit #ab117996) supplemented with anti-proteases. Protein concentration was quantified in each tumor using a Bradford assay. Samples were assayed for pimonidazole (Hypoxyprobe-1) amount using a competitive ELISA assay developed by Omniox and for HIF-1α using the Abcam ELISA kit (ab117996).

In H460 lung carcinoma mouse model maximum oxygenation was achieved between 4 h and 8 h and it correlated with the peak of H-NOX (L144F) tumor accumulation as assessed by ELISA (FIG. 2C).

For assessment of the H-NOX (L144F) tumor accumulation, tumors were harvested at different timepoints after injection. Tumors were homogenized in an extraction buffer (Abcam kit #ab117996) supplemented with anti-proteases and protein concentration was quantified in each tumor using a Bradford assay. PEGylated H-NOX (L144F) concentration was quantified by a sandwich ELISA for H-NOX developed by Omniox and normalized to tumor weight.

While supplemental oxygenation of animals successfully increased oxygenation of mouse tumor tissue at 5-10 mmHg oxygen concentration, it had no effect on regions with lower oxygen levels (<5 mmHg). By contrast, PEGylated trimer Tt H-NOX L144F is capable of increasing oxygenation even in severely hypoxic tumor tissue (<5 mmHg). This is likely due to PEGylated trimer Tt H-NOX L144F's superior tissue penetration that enables oxygen delivery to areas beyond oxygen gradient diffusion limits. Moreover, while maximum supplemental oxygenation of mouse tumors is achieved with exposing animals continuously to 95%-100% breathing oxygen [increasing risk of hyperoxic and inflammatory damage to the normal tissues (Kallet & Matthay, 2013 Respir Care, 58 (1): 123-141; Thiel et al., 2005 PLoS Biol, 3 (6), e174)], single bolus i.v. dose of PEGylated trimer Tt H-NOX L144F can maintain tissue oxygenation for more than 7 hours without increasing oxygen levels in normal tissues. A control Tt H-NOX protein (wild type variant)—that is not capable of releasing oxygen at oxygen concentrations present in hypoxic tissues—did not have any effect on tumor oxygenation (FIG. 3C).

Seven to eight week old Nu/Nu female mice were subcutaneously implanted with 3×10⁶ of H460 human lung cancer cells and monitored until the tumors reached average size of ˜500 mm³ (10-18 days post-implantation of tumor cells). Mice bearing H460 tumors were anesthetized with isoflurane mixed in 20% of oxygen and the OxyLite™ probe (Oxford Optronix, UK) was implanted into H460 subcutaneous xenograft tumors using a micromanipulator. The OxyLite™ consists of the ruthenium chloride dye held in a polymer matrix of 230 μm in diameter at the tip. After equilibration for ˜20-30 minutes, pO₂ was measured using optical fluorescence sensors attached to a four-channel unit. A low starting pO₂ confirmed entry into hypoxic tissue away from neighbouring blood vessels (˜0.2 mmHg; except in FIG. 3D where 5 mmHg). After probe implantation, probe was left for ˜20-30 min in order for pO₂ measurements to stabilize, and mice were given to respire 100% O₂ (FIG. 3B, FIG. 3D) or were injected with PEGylated H-NOX (L144F in FIG. 3A, wt in FIG. 3C) and fluorescent quenching was recorded.

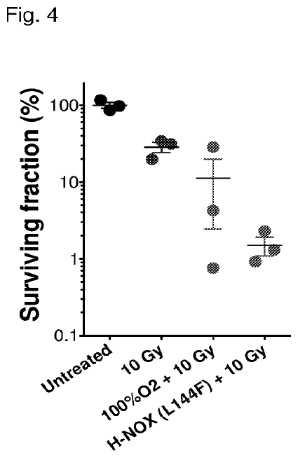

The superior ability of PEGylated trimer Tt H-NOX L144F to deliver oxygen to hypoxic tumor regions relative to the administration of the hyperoxic gas was further demonstrated by more efficient radiation tumor cell kill (FIG. 4).

Mice bearing H460 subcutaneous xenograft tumors (200-350 mm³) were treated with 10 Gy alone or in combination with 650 mg/kg of PEGylated trimer H-NOX (L144F) injected i.v. 7 hours prior to irradiation. Tumors were extracted after irradiation and processed for clonogenic assay. Cell numbers were counted 10-14 days later in triplicate samples from each tumor. Each dot on the graph represents average surviving fraction for one tumor. Mean values +/−SEM (n=3 per experiment). Seven to eight week old Nu/Nu female mice were subcutaneously implanted with 3×10⁶ of H460 human lung cancer cells and monitored until the tumors reached average size of ˜300 mm³ (10-14 days post-implantation of tumor cells). Mice bearing 200-350 mm³ xenograft tumors were irradiated with 10 Gy alone or in combination with intravenous delivery of either a bolus vehicle (formulation buffer: 50 mM succinate, 50 mM NaCl, 3.4 mM EDTA, and 10 mM reduced glutathione at pH 7) or a formulation buffer containing 650 mg/kg of PEGylated H-NOX (L144F). Mice were sacrificed after irradiation and tumors were harvested and processed for an ex-vivo clonogenic assay. Briefly, tumors cells were minced into fine pieces with a scalpel and digested for ˜30 minutes with an enzymatic cocktail containing a mix of collagenase (200 U/ml), hyaluronidase (200 U/ml) and DNAse (10 ml of cocktail/g of tumor). Extracted cells were then counted and seeded at 500/125/25 cells per well for untreated and 2000/500/100 cells per well for irradiated tumor samples in a 6 well plate in duplicate. After 10-12 days, cell colonies were fixed with PFA and stained with crystal violet. The number of clones (over 50 cells) was counted and the plating efficiency was calculated in untreated samples (number of cells counted in well÷number of cells plated×100). Surviving fraction was calculated in all samples (number of cells counted÷plating efficiency×100).

Following PEGylated trimer Tt H-NOX L144F administration, there was >15-fold increase in radiation treatment efficacy in all PEGylated trimer Tt H-NOX L144F-treated tumors that reduced the surviving fraction of tumor cells from 30% in the 10 Gy treatment alone group to <2% in the H-NOX (L144F)-pretreated tumor. In the same experiment, treatment of mice bearing tumors with 100% oxygen showed variable increase in radiation enhancement (˜3 fold) probably resulting from unequal tumor oxygenation between individual tumors likely due to uneven vascular density between tumors.

### Example 2. H-NOX Acts as an Immunoactivator Enhancing Host Anti-Tumor Responses

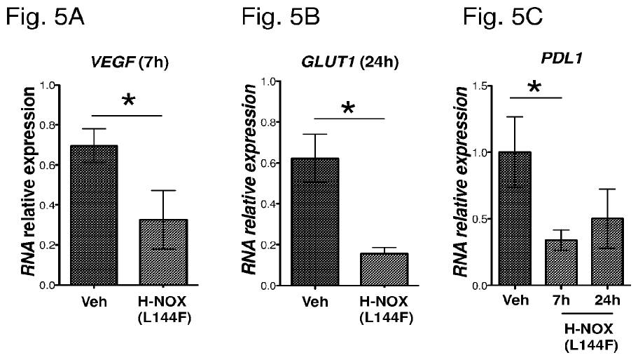

PEGylated trimer Tt H-NOX L144F-induced oxygenation inhibits the HIF-1α pathway (FIG. 2B) and relieves HIF-1α-dependent and HIF-1α-independent tumor immunosuppression. Mice bearing H460 subcutaneous xenograft tumors (200-350 mm³) were either treated with vehicle alone or with PEGylated trimer H-NOX (L144F) and harvested 7, 16 or 24 hours after injection for qRT-PCR analysis. Mean values+/−SEM. N=5-6 per group, *p<0.05 by t-test. Treatment with a single dose of H-NOX resulted in significant downregulation of HIF-1α and its effectors including direct and indirect modulators of the host's immune response: PD/PDL-1 and VEGF signaling, metabolic and growth factor regulators (FIG. 5).

Seven to eight week old Nu/Nu female mice were subcutaneously implanted with 3×10⁶ of H460 human lung cancer cells and monitored until the tumors reached average size of ˜300 mm³ (10-14 days post-implantation of tumor cells). Mice bearing 200-350 mm³ xenograft tumors were injected with either a bolus vehicle (formulation buffer: 50 mM succinate, 50 mM NaCl, 3.4 mM EDTA, and 10 mM reduced glutathione at pH 7) or a formulation buffer containing 650 mg/kg of PEGylated trimer H-NOX (L144F). To prepare samples for qRT-PCR analysis, mice were sacrificed and tumors excised. Total RNA was extracted from tumor samples using the RNeasy kit (QIAGEN) according to the manufacturer instructions. Reverse transcription and real-time PCR (RT-PCR) on a StepOnePlus™ Real-Time PCR System (Applied Biosystems) were performed as described. 25 μL reaction was prepared using 2 μL of cDNA template, 12.5 μL of SYBR® Green PCR Master Mix (Applied Biosystems) and 1 μL of the following sense and antisense primers: VEGF: forward, 5′-CAATCGAGACCCTGGTGGA-3′ (SEQ ID NO:23); reverse, 5′-GCACACACTCCAGGCCCT-3′ (SEQ ID NO:24); Glut1: forward, 5′-CAACCAGACATGGGTCCAC-3′ (SEQ ID NO:25); reverse, 5′-GTTAACGAAAAGGCCCACAGA-3′ (SEQ ID NO:26); PDL1: forward, 5′-GTTGTGGATCCAGTCACCTCT-3′ (SEQ ID NO:27); reverse, 5′-GATTCTCAGTGTGCTGGTCAC-3′ (SEQ ID NO:28); L7: forward, 5′-CAAGGAGGAAGCTTATCTATGAA-3′ (SEQ ID NO:29); reverse, 5′-ATTTGACGAAGGCGAAGAAGCT-3′ (SEQ ID NO:30). Thermocycling conditions were as follows: initial step was 10 min at 95° C., then 40 cycles of 15 s denaturation at 95° C. followed by 1 min annealing and extension at 60° C. Results were analyzed with the StepOne Software v2.0 using the comparative CT method. Transcripts of gene of interest were normalized against the transcripts of the mouse ribosomal protein L7 housekeeping gene, and were presented as fold change relative to the L7 transcript content.

For example, PEGylated trimer Tt H-NOX L144F-mediated downregulation of the HIF-1α and A2AR adenosinergic signaling may result in activation and recruitment of effector T cells to the tumor tissue leading to increased lymphocyte tumor infiltration, decrease in metastatic tumor growth and tumor regression. Furthermore, H-NOX-induced oxygenation of tumors may reduce immunoevasion of tumor cells by inhibiting multiple hypoxia-dependent mechanisms including downregulation of MHC1 and upregulation of PDL-1 expression on tumor cell surface, and activation and recruitment of myeloid-derived suppressor cells (MDSC) including TAMs that directly suppress immune effector cells as well as promote angiogenesis and metastasis (see FIG. 1B). H-NOX treatment may inhibit recruitment of macrophages to TME by downregulating VEGF, CSF1 and other HIF-1-dependent cytokine signaling (Chaturvedi et al., 2014 Proc Natl Acad Sci USA, 111 (20): E2120-2129; Lewis & Hughes, 2007 Breast Cancer Res, 9 (3): 209) as well as HIF-1- and HIF-2-mediated macrophage activation (Fang et al., 2009 Blood, 114 (4): 844-859; Takeda et al., 2010 Genes Dev, 24 (5): 491-501).

Finally, in stimulating host's anti-tumor immune response, H-NOX may act as co-activator enhancing other targeted cancer immunotherapies such as, but not limited to, anti-PD1 (programmed cell death protein 1), anti-PDL-1 (programmed cell death protein ligand 1), anti-CTLA-4 or therapies targeting other immune checkpoints' regulators, anti-cancer vaccines, adoptive immune cell therapies or combinations thereof. For example, H-NOX may be administered to cancer patients prior to and in conjunction with dual PD1/CTLA-4 blockade therapy or in combination with PDL-1 treatment in patients with PDL1+ tumors. It may also act as an adjuvant to other cancer treatments including, but not limited to, chemotherapy, radiation therapy or other non-immune targeted or cell-based therapies that may benefit from active anti-tumor immune defenses. Indeed, H-NOX may synergize with radiation by simultaneously stimulating anti-tumor immune response towards radiation exposed tumor-specific antigens from damaged tumor tissue (Demaria et al., 2005 Int J Radiat Oncol Biol Phys, 63 (3), 655-666) and oxygen-dependent tumor cell killing (Brown, 2010 Int J Radiat Biol, 86 (11), 907-917). During radiotherapy, H-NOX may also act as normal tissue radioprotectant by ameliorating hypoxia resulting from radiation-induced vascular damage.

### Example 3. Measurement of Hypoxia and T Cells in B16F10 and CT26 Subcutaneous Tumors and Intracranial GL261-Luciferase Tumors

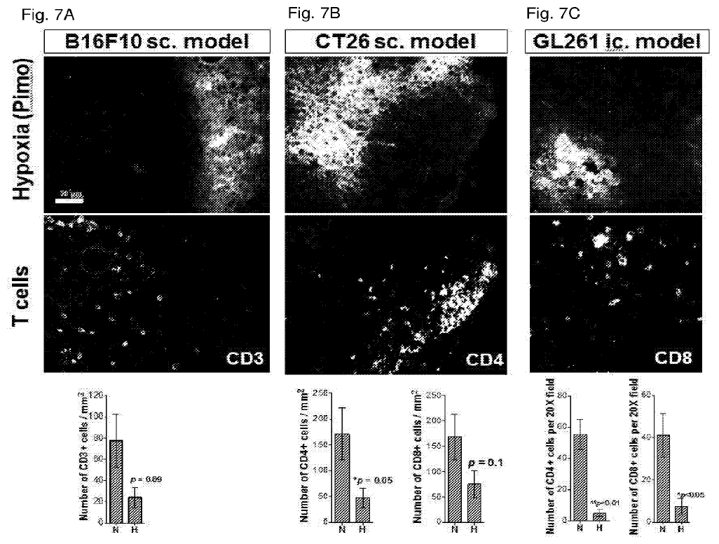

Generation of B16F10 and C′T26 subcutaneous tumors and intracranial GL.261-luciferase tumors. Six to eight week old C57BL/6J female mice were subcutaneously implanted with 1×10⁶ B16F10 mouse melanoma cancer cells on the flank (FIG. 7A). Six to eight week old BALB/c female mice were subcutaneously implanted with 1×10⁶ CT26 colon tumor cells on the flank (FIG. 7B). Male C57BL/6J weighting 20 g were injected with 3×10⁵ GL261-luc cells intracranially into the right caudate nucleus (+0.5 mm A/P, +2.3 mm M/L and −3.2 mm D/V) (FIG. 7C). Intracranial tumors were allowed to grow for 21 days before sacrifice. Subcutaneous tumors were measured 3 times a week using calipers and tumor size was calculated based on the formula: (length×width²)÷0.5. Once the tumors reached an average size of ˜300 mm³ (10-14 days post-implantation of tumor cells), treatment was initiated.

Treatment. Mice bearing 200-400 mm³ subcutaneous tumors or day 21 intracranial tumors were injected with the exogenous hypoxia marker pimonidazole ip. (60 mg/kg, Hypoxyprobe, Burlington Massachusetts) 1-8 hours prior to sacrifice.

Immunohistochemistry. Rodents were euthanized and tumors resected for immunohistochemistry (IHC) assay. Tumors were frozen in OCT and sectioned at 12 μM for IHC processing. Sections were fixed with 4% PFA for 15 minutes at 4° C., then blocked and permeabilized with 5% BSA, 5% goat serum, and 0.1% Tween 20 for 1-2 hours at room temperature. Sections were then incubated with rabbit anti-pimonidazole (Hypoxyprobe, 1:100) (FIGS. 7A, 7B, 7C, top panels) and rat anti-CD3, rat anti-CD4 (Biolegend, 1:50) or rat anti-CD8 (Biolegend, 1:50) antibodies overnight at 4° C. (FIGS. 7A, 7B, 7C, middle panels), followed by anti-rabbit or anti-rat secondary antibodies (1:1000, Jackson Immunoresearch Laboratories, West Grove, PA, USA) for 2 hours at room temperature. The sections were mounted in SlowFade DAPI (Invitrogen). Sections were imaged with an HD Axiolmager Zeiss microscope equipped with a CCD digital camera. Quantification. In each animal, the number of CD3+, CD4+ or CD8+ T cells was counted in pimonidazole-positive and pimonidazole-negative areas in 2-4 pictures per section in 5 tumor sections spanning 1-1.5 mm of the tumor thickness. The sum of CD4+ and CD8+ cells in each area was divided by the sum of pimonidazole-postive and pimonidazole-negative areas to obtain the total number of T cells per mm² of tumor tissue (FIGS. 7A, 7B, and 7C, bottom panels). Hypoxic regions of tumors (H) showed 2.5 to over 10-fold less T cells than normoxic regions (N).

### Example 4. H-NOX Treatment of Hypoxic Tumors

Generation of B16F10 subcutaneous tumors. Six to eight week old C57BL/6J female mice were subcutaneously implanted with 1×10⁶ of B16F10 mouse melanoma cancer cells on the flank. Tumors were measured 3 times a week using calipers and tumor size was calculated based on the formula: (length×width²)÷0.5. Once the tumors reached an average size of ˜300 mm³ (10-14 days post-implantation of tumor cells), treatment was initiated.

Treatment. Mice bearing 200-400 mm³ tumors were randomized in each treatment group based on tumor size and injected intratumorally with vehicle (formulation buffer: 50 mM succinate, 50 mM NaCl, 3.4 mM EDTA, and 10 mM reduced glutathione at pH 7) or 100 μl of formulation buffer containing 2 mg of PEGylated H-NOX (L144F). One hour prior to vehicle or H-NOX treatment, mice were injected with the exogenous hypoxia marker pimonidazole ip. (60 mg/kg, Hypoxyprobe, Burlington Massachusetts).

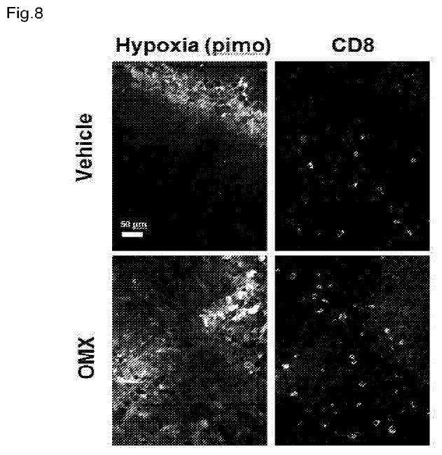

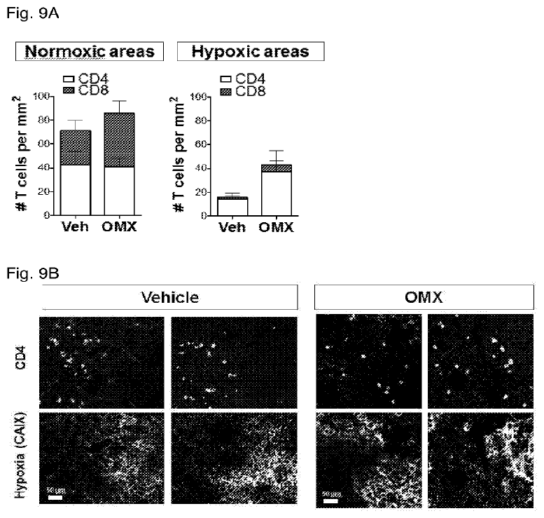

Immunohistochemistry. 6 hours after H-NOX injection, rodents were euthanized and tumors resected for immunohistochemistry (IHC) assay. Tumors were frozen in OCT and sectioned at 12 μM for IHC processing. Sections were fixed with 4% PFA for 15 minutes at 4° C., then blocked and permeabilized with 5% BSA, 5% goat serum, and 0.1% Tween 20 for 1-2 hours at room temperature. Sections were then incubated with rabbit anti-pimonidazole (Hypoxyprobe, 1:100) or rabbit anti-carbonic anhydrase IX (CAIX, Novus Biological 1:1000) and rat anti-CD4 (Biolegend, 1:50) or rat anti-CD8 (Biolegend, 1:50) antibodies overnight at 4° C., followed by anti-rabbit or anti-rat secondary antibodies (1:1000, Jackson Immunoresearch Laboratories, West Grove, PA, USA) for 2 hours at room temperature. The sections were mounted in SlowFade DAPI (Invitrogen). Sections were imaged with an HD Axiolmager Zeiss microscope equipped with a CCD digital camera (FIGS. 8 and 9B).

Quantification. In each animal, the number of CD4+ and CD8+ T cells was counted in pimonidazole-positive, pimonidazole-negative, CAIX-positive and CAIX-negative areas in 4 pictures per section in 5 tumor sections spanning 1-1.5 mm of the tumor thickness. The sum of CD4+ and CD8+ cells in each area was divided by the sum of pimonidazole-positive, pimonidazole-negative, CAIX-positive and CAIX-negative areas to obtain the total number of T cell per mm² of tumor tissue. Results shown in FIGS. 8 and 9A demonstrate that OMX treatment as compared to the vehicle control (formulation buffer) enhances accumulation of CD4+ and CD8+ lymphocytes in previously pimondazole-negative (FIG. 8) ro CAIX-negative (FIG. 9B) labeled hypoxic regions of the tumors.

### Example 5. Measurement of Tumor Hypoxia and Tumor Vessels

Generation of H460, B16F10 and (′T26 subcutaneous tumors and intracranial GL.261-luciferase tumors. Seven to eight week old Nu/Nu female mice were subcutaneously implanted with 3×10⁶ of H460 human lung cancer cells in the hind limb. Six to eight week old C57BL/6J female mice were subcutaneously implanted with 1×10⁶ B16F10 mouse melanoma cancer cells on the flank. Six to eight week old BALB/c female mice were subcutanously implanted with 1×10⁶ CT26 colon tumor cells on the flank. Male C57BL/6J weighting 20 g were injected with 3×10⁵ GL261-luc cells intracranially into the right caudate nucleus (+0.5 mm A/P, +2.3 mm M/L and −3.2 mm D/V). Intracranial tumors were allowed to grow for 21 days before sacrifice. Subcutaneous tumors were measured 3 times a week using calipers and tumor size was calculated based on the formula: (length×width²)÷0.5. Once the tumors reached an average size of ˜300 mm³ (10-14 days post-implantation of tumor cells), treatment was initiated. Treatment. Mice bearing 200-400 mm³ subcutaneous tumors or day 21 intracranial tumors were injected with the exogenous hypoxia marker pimonidazole ip. (60 mg/kg, Hypoxyprobe, Burlington Massachusetts) 1-8 hours prior to sacrifice.

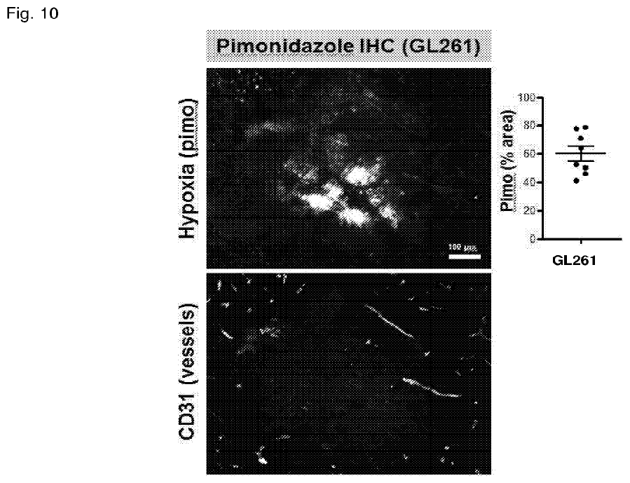

Immunohistochemistry and ELISA. Rodents were euthanized and tumors resected for immunohistochemistry (IHC) and ELISA assays. For ELISA, B16F10, CT26 and H460 tumors were homogenized in an extraction buffer (Abcam kit #ab117996) supplemented with anti-proteases. Protein concentration was quantified in each tumor using a Bradford assay and samples were assayed for hypoxia levels using a competitive Pimonidazole (Hypoxyprobe-1, Hypoxyprobe, Burlington Massachusetts) ELISA assay. For IHC, GL261 tumors were frozen in OCT and sectioned at 12 μM for IHC processing. Sections were fixed with 100% methanol for 20 minutes at −20° C., then blocked and permeabilized with 5% BSA, 5% goat serum, and 0.1% Tween 20 for 1-2 hours at room temperature. Sections were then incubated with rabbit anti-pimonidazole (Hypoxyprobe, 1:100) and rat anti-CD31 (BD Bioscience, 1:50) antibodies overnight at 4° C., followed by anti-rabbit or anti-rat secondary antibodies (1:1000, Jackson Immunoresearch Laboratories, West Grove, PA, USA) for 2 hours at room temperature. The sections were mounted in SlowFade DAPI (Invitrogen). Sections were imaged with an HD Axiolmager Zeiss microscope equipped with a CCD digital camera (FIG. 10).

Quantification. For the pimonidazole ELISA, quantification of the IC₅₀ values (“Kd”) were performed with a 5-parameter fit of the standard curve and values were normalized according to the protein concentration in each sample. For GL261 IHC, in each animal, the percent of pimonidazole+ area within the tumor tissue was determined using ImageJ (1-2 pictures per section in 5 tumor sections spanning 1 mm of the tumor thickness) (FIG. 10). These data show that while there is a range in the levels of hypoxia between individual animals and between tumor types, its presence is significant in a majority of the tumors of the examined sizes.

### Example 6. Measurement of H-NOX Accumulation in Tumors

Generation of B16F10 and CT26 subcutaneous tumors and intracranial GL261-luciferase tumors. Six to eight week old C57BL/6J female mice were subcutaneously implanted with 1×10⁶ B16F10 mouse melanoma cancer cells on the flank. Six to eight week old BALB/c female mice were subcutanously implanted with 1×10⁶ CT26 colon tumor cells on the flank. Male C57BL/6J weighting 20 g were injected with 3×10⁵ GL261-luc cells intracranially into the right caudate nucleus (+0.5 mm A/P, +2.3 mm M/L and −3.2 mm D/V). Intracranial tumors were allowed to grow for 21 days before sacrifice. Subcutaneous tumors were measured 3 times a week using calipers and tumor size was calculated based on the formula: (length×width²)÷0.5. Once the tumors reached an average size of ˜300 mm³ (10-14 days post-implantation of tumor cells), treatment was initiated.

Treatment. Mice bearing 200-400 mm³ subcutaneous tumors were randomized in each treatment group based on tumor size and injected intravenously (650 mg/kg), subcutaneously (650 mg/kg), or intratumorally (2 mg, 100 μl) with vehicle (formulation buffer: 50 mM succinate, 50 mM NaCl, 3.4 mM EDTA, and 10 mM reduced glutathione at pH 7) or formulation buffer containing PEGylated H-NOX (L144F). Mice bearing day 21 intracranial tumors were randomized in each treatment group based on bioluminescent signal measured with the Xenogen IVIS spectrum and injected intravenously with formulation buffer alone or containing 750 mg/kg of H-NOX (L144F).

Measurement of PEGylated H-NOX (1.144F) accumulation in subcutaneous tumor tissue. Tumors were harvested 6 h (B16F10) or 8 h (CT26) after H-NOX or vehicle injection. Tumors were homogenized in an extraction buffer (Abcam kit #ab117996) supplemented with anti-proteases and protein concentration was quantified in each tumor using a Bradford assay. PEGylated H-NOX (L144F) concentration was quantified by a sandwich ELISA ELISA (detection sensitivity at 1 ng/ml) for H-NOX and normalized to tumor weight to express H-NOX amount in μg/g tumor tissue. Quantification of H-NOX levels in tumor lysates was determined by 5-parameter fit of the standard curve.

Biodistribution of H-NOX (1.144F) in GL261 by IHC. 2 h after H-NOX injection, rodents were euthanized and tumors resected for immunohistochemistry (IHC) assay. Tumors were frozen in OCT and sectioned at 12 μM for IHC processing. Sections were fixed with 100% methanol for 20 minutes at −20° C., then blocked and permeabilized with 5% BSA, 5% goat serum, and 0.1% Tween 20 for 1-2 hours at room temperature. Sections were then incubated with rabbit anti-H-NOX (1:500, custom-made rabbit polyclonal produced by AnaSpec Inc, Fremont, CA) and rat anti-CD31 (BD Bioscience, 1:50) antibodies overnight at 4° C., followed by anti-rabbit or anti-rat secondary antibodies (1:1000, Jackson Immunoresearch Laboratories, West Grove, PA, USA) for 2 hours at room temperature. The sections were mounted in SlowFade DAPI (Invitrogen). Sections were imaged with an HD Axiolmager Zeiss microscope equipped with a CCD digital camera. In each animal, the percent of H-NOX-positive area within the tumor tissue was determined using ImageJ (1-2 pictures per section in 5 tumor sections spanning 1 mm of the tumor thickness).

### Example 7. Measurement of Hypoxia and T Cells in Canine Oral Melanoma Tumors

Canine oral melanoma. Pet dogs with oral melanoma tumors were recruited for the study with owners' consent and injected intravenously (slow infusion) with PEGylated H-NOX (L144F) 4 h prior to surgery. Tissue extracted from surgery was analyzed by IHC.

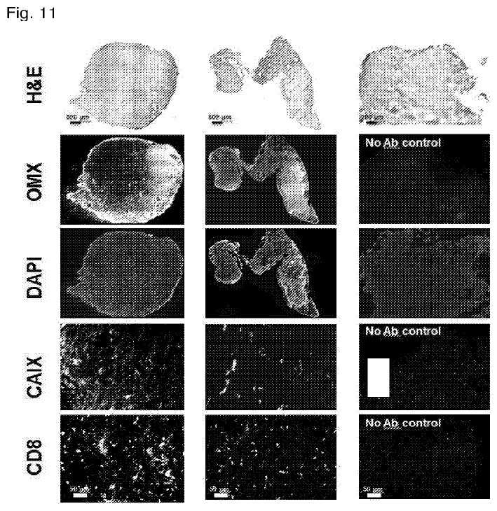

Immunohistochemistry. 4 hours after H-NOX injection, tumors were resected, frozen in OCT and sectioned at 12 μM for IHC processing. Sections were fixed with 4% PFA for 15 minutes at 4° C., then blocked and permeabilized with 5% BSA, 5% goat serum, and 0.1% Tween 20 for 1-2 hours at room temperature. Sections were then incubated with rabbit anti-carbonic anhydrase IX (CAIX, Novus Biological 1:1000) or rabbit anti-H-NOX (1:500, custom-made rabbit polyclonal produced by AnaSpec Inc, Fremont, CA) and rat anti-CD4 (Abd Serotech, 1:50) or rat anti-CD8 (Abd Serotech, 1:50) antibodies overnight at 4° C., followed by anti-rabbit or anti-rat secondary antibodies (1:1000, Jackson Immunoresearch Laboratories, West Grove, PA, USA) for 2 hours at room temperature. The sections were mounted in SlowFade DAPI (Invitrogen). Sections were imaged with an HD Axiolmager Zeiss microscope equipped with a CCD digital camera (FIG. 11). Images revealed presence of high lymphocyte numbers in tumor regions that expressed hypoxia marker CAIX indicative of hypoxic state prior to OMX administration suggesting that OMX treatment relieved immunosuppressive microenvironment and allowed lymphocyte infiltration.

### Example 8. Correlation of Tumor Volume, Tumor Hypoxia and Reduced T Cell Infiltration

4T1-Luc Tumor Model. 8 week-old female BALB/c mice were purchased from Charles River Labs. Luciferase-expressing 4T1 mouse breast tumor cells (4T1-Fluc-Neo; Imanis Life Sciences) were grown in RPMI medium supplemented with 10% fetal bovine serum, 1×penicillin/streptomycin, and 0.7 mg/ml G-418 (InvivoGen). Cells were trypsinized and resuspended in a 50:50 mixture of medium: Matrigel (Corning), and 2×10⁵ cells in 100 μl volume was injected subcutaneously into mice. At day 10 and day 14 post-implantation, tumors were measured and volumes calculated (length×width×height×0.523), mice were injected simultaneously with 120 mg/kg pimonidazole i.p. (PIMO, Hypoxyprobe) and 30 mg/kg EF5 i.v. (Hypoxia Imaging Center), sacrificed 90 min post-PIMO/EF5 injection, and tumors were harvested. Harvested tumors were frozen in OCT for immunostaining, as well as dissociated into single cells using a gentleMACS dissociator followed by incubation with 0.75 mg/ml collagenase/dispase (Roche) at 37° C. with shaking for 45 min. Dissociated cells were passed through 70 μm filters.

Flow Cytometry. Unfixed dissociated cells were stained with antibodies for T cells (hamster anti-mouse CD3-AlexaFluor 488, clone 145-2C11, eBioscience; rat anti-mouse CD4-APC, clone RM4-5, BD Biosciences; rat anti-mouse CD8-PE, clone 53-6.7, BD Biosciences), and flow cytometry was performed using a FACSCalibur. Spleens were used as T cell positive controls for gating purposes. Also after filtration of dissociated cells through 70 μm filters, cells were stained with viability dye 570 (BD Biosciences), fixed with formalin and methanol, stained with antibodies for hypoxia markers (rabbit anti-pimonidazole, Hypoxyprobe, followed by donkey anti-rabbit AlexaFluor 647; mouse anti-EF5 conjugated to AlexaFluor 488, Hypoxia Imaging Center), and analyzed on a FACSCalibur. Flow cytometry data were analyzed using FlowJo.

Immunofluorescence Staining. Frozen sections were cut at 10 μm, fixed with 4% PFA, stained with primary antibodies (rat anti-mouse CD4, rat anti-mouse CD8, rabbit anti-PIMO), followed by secondary antibodies (donkey anti-rat AlexaFluor 594, donkey anti-PIMO AlexaFluor 488), and counterstained with DAPI.

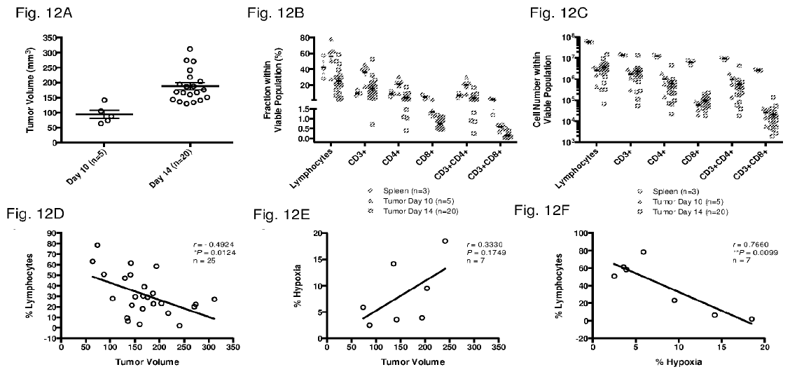

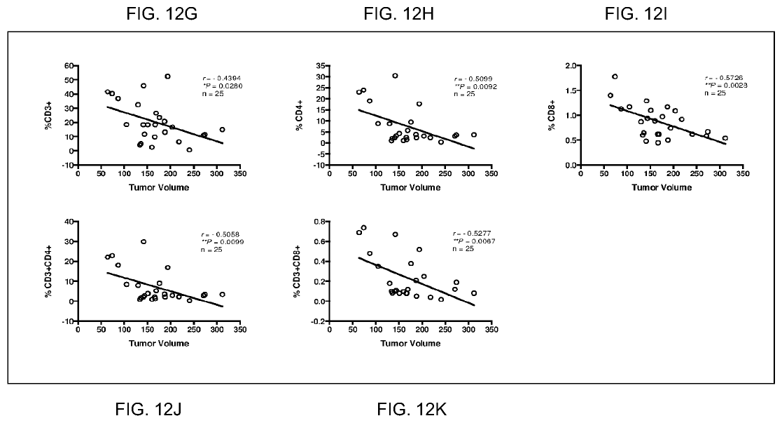

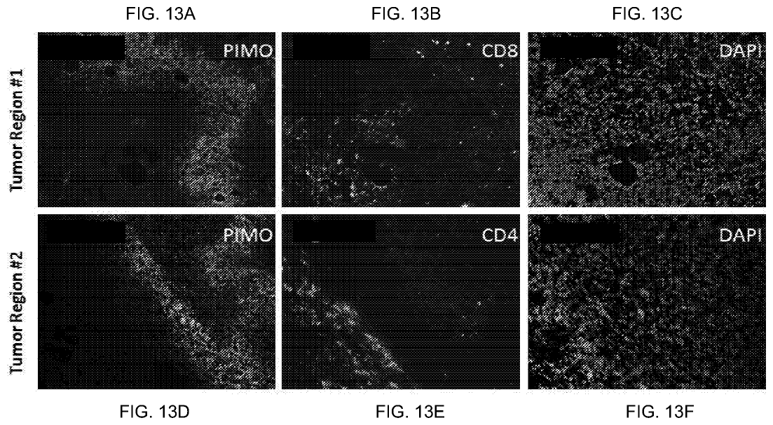

As shown in FIGS. 13A-13K, larger tumor size correlates with enhanced hypoxia and reduced lymphocyte infiltration in subcutaneous 4T1-Luc syngeneic tumors. FIG. 12A shows tumor volumes on day 10 and day 14 post-implantation. FIG. 12B shows the fraction of lymphocytes within the viable cell population and FIG. 12C shows the absolute lymphocyte cell numbers within the viable population. Negative correlations between tumor volume and percentage lymphocytes (FIG. 12D) and between percentage hypoxia and percentage lymphocytes (FIG. 12F) were demonstrated whereas the relationship between tumor volume and percentage hypoxia showed a positive correlation (FIG. 12E). Negative correlations were also seen between tumor volume and percentage CD3-positive T cells (FIG. 12G), between tumor volume and percentage CD4-positive T cells (FIG. 12H), between tumor volume and percentage CD8-positive T cells (FIG. 12I), between tumor volume and percentage CD3-CD4-double-positive T cells (FIG. 12J), and between tumor volume and percentage CD3-CD8-double-positive T cells (FIG. 12K).

FIGS. 13A-13F show that hypoxic tumor regions are immunosuppressive and exhibit reduced T cell infiltration in subcutaneous 4T1-Luc syngeneic mouse tumors.

SEQUENCES

TT. WT
ATGAAGGGGACAATCGTCGGGACATGGATAAAGACCCTGAGGGACCTTTACGGGA
ATGATGTGGTTGATGAATCTTTAAAAAGTGTGGGTTGGGAACCAGATAGGGTAAT
TACACCTCTGGAGGATATTGATGACGATGAGGTTAGGAGAATTTTTGCTAAGGTG
AGTGAAAAAACTGGTAAAAATGTCAACGAAATATGGAGAGAGGTAGGAAGGCAGA
ACATAAAAACTTTCAGCGAATGGTTTCCCTCCTATTTTGCAGGGAGAAGGCTAGT
GAATTTTTTAATGATGATGGATGAGGTACACCTACAGCTTACCAAGATGATAAAA
GGAGCCACTCCTCCAAGGCTTATTGCAAAGCCTGTTGCAAAAGATGCCATTGAAA
TGAGTACGTTTCTAAAAGAAAGATGTACGATTACTTTTTAGGGCTTATAGAGGGT
AGTTCTAAATTTTTCAAGGAAGAAATTTCAGTGGAAGAGGTCGAAAGAGGCGAAA
AAGATGGCTTTTCAAGGCTAALAGTCAGGATAAAATTTATTAAACCCCGTTTTTG
AGTGA (SEQ ID NO: 1)
MKGTIVGTWIKTLRDLYGNDVVDESLKSVGWEPDRVITPLEDIDDDEVRRIFAKV
SEKTGKNVNEIWREVGRQNIKTFSEWFPSYFAGRRLVNFLMMMDEVHLQLTKMIK
GATPPRLIAKPVAKDAIEMEYVSKRKMYDRYFLGLIEGSSKFFKEEISVEEVERG
EKDGFSRLKVRIKFKNPVFE (SEQ ID NO: 2)
foldon domain
GGTTATATTCCTGAAGCTCCAAC4AGATGCAAGCTTACGTTCGTAAAGATGGCGA
ATGGGTATTACTTTCTACCTTTTTA (SEQ ID NO: 3)
GYIPEAPRDGQAYVRKDGEWVLLSTFL (SEQ. ID NO: 4)
L2 WT
ATGATGTCTATGAAAGGAATCATATTCAACGAATTTCTCAATTTTGTAGAAAAAA
GTGAATCCTACACCCTGGTAGATCAAATTATTATGGATAGTCATTTGAAGTCCCA
TGGTGCCTACACGTCTATCGGTACATACTCTGCCAAAGAATTATTTCAATTGGTT
AAAGCGCTTGCTATGAAAAATGGCAAACCAACATCAGTGATTTTACAAGAATATG
GTGAGTATTTGTTTGAGGTTTTTGGAAAAAAATATCCTCAATTTTTCAGGGAAAA
AAAGTCGGTGTTTCAATTTTTGGAAGCGCTTGAAACACATATTCATTTCGAAGTG
AAAAAATTGTATGAGTATACTGAACTACCCCATTTTGAATGCCAATATCACAGTG
AAAATCAAATGGAAATGATTTACACTTCTTCGCGTCCTTTGGCCGATTTTGCGGA
AGGTTTAATAAAAGGTTGTATTAAATATCATAAAGAAAACATGACTATTGTTCGT
GAAAATCTGCCTGCAAAAACAGGCTTTAAGGTAAGATTTGTATTAACAAAAGGCG
ATCCTGATGAGTGA (SEQ ID NO: 9)
MMSMKGIIFNEFLNFVEKSESYTLVDQIIMDSHLKSHGAYTSIGTYSPKELFQLVKA
LAMKNGKPTSVILQEYGEYLFEVFAKKYPQFFREKKSVFQFLEALETHIHFEVKKLY
DYTELPHFECQYHSQNQMEMIYTSSRPLADFAEGLIKGCIKYHKENMTIVRENLPAK
TGFKVRFVLTKGDPDE (SEQ ID NO: 10)
L1 WT
ATGAAAGCTATCGTTTTTACCTCCTTAAATGACATGATTATAGAACAATTTGGC
ATAGAAACCTGGGACCAACTCGTATCCTCACTAGACCETCCAAGTGGEGGAAGT
TATACAGCAGGCGGCACTTACTGGGATACAGAATTTCAGCAATTGATTAAGGCC
ATTGCGAAGAGGACCAATCAGGACGCTTCTGTTTTTTTAGAGGCCTTTGGTGAA
TACATGTTTCCTATCTTATCGAGTAAGTGCGCAATTTTTTTAAAAAAGGACATG
ACATTAAAAGAATTTTTAAAAAGCATTGATGGAACAATTCATGTGGAAGTAGAA
AAGTTATACCCAGATGAAACATTACCTACCATTAGCTATGAAGAGCCTGCTGCA
AACCAALTTGTTATTGGTATCGATCGCATAGAAGACTCTGTCATTTTGCAATGG
GGCTECATCCAGGGAGCAGCGCAACATTTTAAAAAGAAAATTACCATTAAGCAG
ACTCACTGCATGTTAAAAAAAGATGATCATTGTCGTTTGGAGATTACCTTTGAG
TGA (SEQ ID NO: 11)
MKGIVFTSLNDMIIEQFGIETWDQLVSSLDLPSGGSYTAGGTYSDTEFQQLIKAIAKR
TNQHASVFLEAFGEYMFPILSSKCAIFLKKDMTLKEFLKSIDGTIHVEVEKLYPDETL
PTISYEEPAANQLVMVYRSHRRLCHFAMGLIQGAAQHFKKKITIKQTHCMLKKDDHCR
LEITFE (SEQ ID NO: 12)
Homo sapiens WT (1-385)
ATGTACGGATTTGTGAATCACGCCCTGGAGTTGCTGGTGATCCGCAATTACGGC
CCCGAGGTGTGGGAAGACATCAAAAAAGAGGCACAGTTAGATGAAGAAGGACAG
TTTCTTGTCAGAATAATATATGATGACTCCAAAACTTATGATTTGGTTGCTGCA
AGCAAAGTCCTCAATCTCAATGCTGGAGAAATCCTCCAAATGTTTGGGAAGATG
TTTTTCGTCTTTTGCCAAGAATCTGGTTATGATACAATCTTGCGTGTCCTGGGC
TCTAATGTCAGAGAATTTCTACAGAACCTTGATGCTCTGCACGACCACCTTGCT
ACCATCTACCCAGGAATGCGTGCACCTTCCTTTAGGTGCACTGATGCAGAAAAG
GGCAAAGGACTCATTETGCACTACTACTCAGAGAGAGAAGGACTTCAGGATATT
GTCATTGGAATCATCAAAACAGTGGCACAACAAATCCATGGCACTGAAATAGAC
ATGAAGGTTATTCAGCAAAGAAATGAAGAATGTGATLATACTCAATTTTTAATT
GAAGAAAAAGAGTCAAAAGAAGAGGATTTTTATGAAGATCTTGACAGATTTGAA
GAAAATGGTACCCAGGAATCACGCATCAGCCCATATACATTCTGCAAAGCTTTT
CCTTTTCATATAATATTTGACCGGGACCTAGTGGTCACTCAGTGTGGCAATGCT
ATATACAGAGTTCTCCCCCAGCTCCAGCCTGGGAATTGCAGCCTTCTGTCTGTC
TTCTCGCTGGTTCGTCCTCATATTGATATTAGTTTCCATGGGATCCTTTCTCAC
ATCAATACTGTTTTTGTATTGAGAAGCAAGGAAGGATTGTTGGATGTGGAGAAA
TTAGAATGTGAGGATGAACTGACTGGGACTGAGATCAGCTGCTTACGTCTCAAG
GGTCAAATGATCTACTTACCTGAAGCAGATAGCATACTTTTTCTATGTTCACCA
AGTGTCATGAACCTGGACGATTTGACAAGGAGAGGGCTGTATCTAAGTGACATC
CCTCTGCATGATGCCACGCGCGATCTTGTTCCTGAGAACAATTTAGAGAGGAAT
ACAAACTCACCCAAGAACTGGAAATCCTCACTGACAGGCTACAGCTCAGCTTAA
GAGCCCTGGAAGATTGA (SEQ ID NO: 13)
MYGFVNHALELLVIRNYGPEVWEDIKKEAQLDEEGQFLVRIIYDDSKTYDLVAAAS
KVLNLNAGEILQMFGKMFFVFCQESGYDTILRVLGSNVREFLQNLDALHDHLATIY
PGMRAPSFRCTDAEKGKGLILHYYSEREGLQDIVIGIIKTVAQQIHGTEIDMKVIQ
QRNEECDHTQFLIEEKESKEEDFYEDLDRFEENGTQESRISPYTFCKAFPFHIIFD
RDLVVTQCGNAIYRVLPQLQPGNCSLLSVFSLVRPHIDISFHGILSHINTVFVLRS
KEGLLDVEKLECEDELTGTEISCLRLKGQMIYLPEADSILFLCSPSVMNLDDLTRR
GLYLSDIPLHDATRDLVLLGEQFREEYKLTQELEILTDRLQLTLRALED (SEQ
ID NO: 14)
Homo sapiens β2 (1-217)
ATGTATGGATTCATCAACACCTGCCTGCAGTCTCTTGTGACAGAGAAATTTGGT
GAGGAGACATGGGAGAAGCTGAAGGCTCCTGCAGAAGTGCAAGATGTCTTCATG
ACCTACACCGTGTATGATGACATCATCACCATTAAGCTCATCCAAGAAGCCTGC
AAGGTTCTGGATGTGTCCATGGAAGCCATTCTGAAGCTCTTTGGCGAATACTTC
TTTAAGTTCTGTAAGATGTCTGGCTATGACAGGATGCTGCGGACACTTGGAGGA
AATCTCACCGAGTTTATTGAAAACCTAGATGCACTCCACAGTTACCTGGCACTG
TCCTATCAGGAAATGAACGCACCATCCTTTCGAGTGGAGGAAGGAGCTGACGGG
GCCATGCTTCTCCACTACTACTCAGACAGACATGGTCTGTGTCACATTGTACCA
GGTATCATTGAAGCTGTGGCCAAGGACTTCTTTGACACTGATGTGGCCATGAGT
ATCCTGGATATGAACGAAGAGGTGGAAAGGACAGGGAAGAAAGAACATGTTGTG
TTTCTGTTTCGTTTCAGAAGGCTCACAGACAGATAAGAGGAGCAAAGGCAAGCC
GGCCACAAGGCAGTGAGGACAGCCAGGCAGACCAGGAGGCTCTCCAGGGAACAC
TCCTT (SEC ID NO: 15)
MYGFINTCLQSLVTEKFGEEETWEKLKAPAEVQDVFMTYTVYDDIITIKLIQEACK
VLDVSMEAILKLFGEYFFKFCKMSGYDRMLRTLGGNLTEFIENLDALHSYLALSYQ
EMNAPSFRVEEGADGAMLLHYYSDRHGLCHIVPGIIEAVAKDFFDTDVAMSILDMN
EEVERTGKKEHVVFLVVQKAHRQIRGAKASRPQGSEDSQADQEALQGTLL (SEQ
ID NO: 16)
Rattus norvegicus β1 (1-385)
ATGTACGGTTTTCTCAACCATGCCCTGGAGCTGCTGGTGATCCGCAATTACGGT
CCCGAGTTGTGGGAAGACATCAAAAAAGAGGCGCAGCTGGATGAAGAAGGCCAG
TTTCTTGTGAGAATAATCTACGATGATTCCAAAACCTATGACTTGGTGGCTGCT
GCGAGCAAAGTCCTCAACCTCAATGCTGGTGAAATCCTGCAGATGTTTGGGAAG
ATGTTTTTCGTCTTCTGTCAAGAGTCTGGCTATGATACCATCTTGCGTGTCCTC
GCCACCATATACCCAGGGATGCGCGCACCTTCCTTCCGGTGCACCGATGCAGAA
AAAGGCAAAGGGCTCATTCTGCACTACTACTCGGAAAGAGAGGGGCTTCAGGAC
ATTGTGATCGGGATTATCAAGACTGTAGCTCAACAGATCCATGGCACTGAGATA
GACATGAAGGTTATTCAGCAAAGAAGTGAAGAATGTGATCATACCCAATTTTTA
ATTGAAGAAAAAGAATCAAAAGAAGAGGATTTTTATGAAGATCTGGACAGGTTT
GAAGAGAACGGTACCCAGGACTCCCGTATCAGCCCGTACACCTTCTGCAAAGCG
TTTCCTTTTCACATCATATTTGACCGGGACCTAGTAGTCACGCAGTGTGGAAAT
GCTATCTACAGAGTGCTCCCCCAGCTCCAGCCTGGGAAGTGCAGCCTTCTGTCT
GTCTTCTCTCTGGTCCGCCCTCATATTGACATCAGTTTCCACGGGATTCTTTCA
CACATCAATACCGTCTTTGTACTGAGAAGCAAGGAAGGGTTGCTGGATGTTGAG
AAACTTGAATGTGAGGATGAACTGACTGGGGCAGAGATTAGCTGCCTCCGTCTC
AAAGGCCAAATGATCTATTTACCGGAAGCAGATAGCATCCTCTTCCTCTGTTCA
CCAAGTGTGATGAACTTGGATGACCTAACAAGAAGAGGCCTGTACCTGAGTGAC
ATCCCTCTCCATGATGCTACACGAGACCTGGTCCTTTTGGGAGAACAGTTCCGG
GAGGAGTACAAACTGACACAAGAGCTGGAAATCCCTCACAGACAGGCTGCAGCT
CACACTGAGGGCTTTGGAGGATTGA (SEQ ID NO: 17)
MYGFVNHALELLVIRNYGPEVWEDIKKEAQLDEEGQFLVRIIYDDSKTYDLVAAAS
KVLNLNAGEILQMFGKMFFVFCQESGYDTILRVLGSNVREFLQNLDALHDHLATIY
PGMRAPSFRCTDAEKGKLILHYYSEREGLQDIVIGIIKTVAQQIHGTEIDMKVIQQ
RSEECDHTQFLIEEKESKEEDFYEDLDRFEENGTQDSRISPYTFCKAFPFHIIFDR
DLVVTQCGNAIYRVLPQLQPKCSLLSVSFLVRPHIDISFHGILSHINTVFVFRSKE
GLLDVEKLECEDELTGAEISCLRLKGQMIYLPEADSILFLCSPSVMNLDDLTRRGL
YLSDIPLHDATRDLVLLGEQFREEYKLTQELEILTDRLQLTLRALED (SEQ ID
NO: 18)
Rattus norvegicus β1 (1-385)
ATGTACGGTTTTGTGAACCATGCCCTGGAGCTGCTGGTGATCCGCAATTACGGT
CCCGAGGTGTGGGAAGACATCAAAAAAGAGGCGCAGCTGGATGAAGAAGGCCAG
TTTCTTGTGAGAATAATCTACGATGATTCCAAAACCTATGACTTGGTGGCTGCT
GCGAGCAAAGTCCTCAACTCAATGCTGGTGAAATCCTGCAGATGTTTGGGAGAG
TGTTTTTCGTCTTCTGTCAAGAGTCTGGCTATGATACCATCTTGCGTGTCCTGG
GATCTAATGTCAGGGAGTTTTTGCAGAACCTCGACGCCCTGCACGACCACCTCG
CCACCATCTACCCAGGGATGCGCGCACCTTCCTTCCGGTGCACCGATGCAGAAA
AAGGCAAAGGGCTCATTCTGCACTACTACTCGGAAAGAGAGGGGCTTCAGGACA
TTGTGATCGGGATTATCAAGACTGTAGCTCAACAGATCCATGGCACTGAGATAG
ACATGAAGGTTATTCAGCAAAGAAGTGAAGAATGTGATCATACCCAATTTTTAA
TTGAAAAAAAGAATCAAAAGAAGAGGATTTTTATGAAGATCTGGACAGGTTTGA
AGAGAACGGTACCCAGGACTCCCGTATCAGCCCGTACACCTTCTGCAAAGCGTT
TCCTTTTCACATCATATTTGACCGGGACCTAGTAGTCACGCAGTGTGGAAATGC
TATCTACAGAGTGCTCCCCCAGCTCCAGCCTGGGAAGTGCAGCCTTCTGTCTGT
CTTCTCTCTGGTCCGCCCTCATATTGACATCAGTTTCCACGGGATTCTTTCACA
CATCAATACCGTCTTTGTACTGAGAAGCAAGGAAGGGTTGCTGGATGTTGAGAA
ACTTGAATGTGAGGATGAACTGACTGGGGCAGAGATTAGCTGCCTCCGTCTCAA
AGGCCAAATGATCTATTTACCGGAAGCAGATAGCATCCTCTTCCTCTGTTCACC
AAGTGTGATGACTTGGATGACCTAACAAGAAGAGGCCTGTACCTGAGTGACATC
CCTCTCCATGATGCTACACGAGACCTGGTCCTTTTGGGAGAACAGTTCCGGGAG
GAGTACAAACTGACACAAGAGCTGGAAATCCTCACAGACAGGCTGCAGCTCACA
CTGAGGGCTTTGGAGGATTGA (SEQ ID NO: 19)
MYGFVNHALELLVIRNYGPEVWEDIKKEAQLDEEGQFLVRIIYDDSKTYDLVAAAS
KVLNLNAGEILQMFGKMFFVFCQESGYDTILRVLGSNVREFLQNLDALHDHLATIY
PGMRAPSFRCTDAEKGKGLILHYYSEREGLQDIVIGIIKTVAQQIHGTEIDMKVIQ
QRSEECDHTQFLIEEKESKEEDFYEDLDRFEENGTQDSRISPYTFCKAFPFHIIFD
RDLVVTQCGNAIYRVLPQLQPGKCSLLSVFSLVRPHIDISFHGILSHINTVFVLRS
KEGLLDVEKLECEDELTGAEISCLRLKGQMIYLPEADSILFLCSPSVMNLDDLTRR
GLYLSDIPLHDATRDLVLLGEDQFREEYKLTQELEILTDRLQLTLRALED (SEQ ID
NO: 20)
Rattus norvegicus β2
ATGTATGGATTGATCAACACCTGCCTGCAGTCTCTTGTGACAGAGAAATTTGGT
GAGGAGACATGGGAGAAGCTGAAGGCTCCTGCAGAAGTGCAAGATGTCTTCATG
ACCTACACCGTGTATGATGACATCATCACCATTAAGCTCATCCAAGAAGCCTGC
AAGCTTCTCGATGTGTCCATGGAAGCCATTTCTGAAGCTCTTTGGCGAATACTT
CTTTAAGTTCTGTAAGATGTCTGGCTATGACAGGATGCTGCGGACACTTGGAGG
AAATCTCACCGAGTTTATTGAAAACCTAGATCCACTCCACAGTTACCTGCCACT
CTCCTATCAGGAAATGAACGCACCATCCTTTCGAGTGGAGGAAGGAGCTGACGG
GGCGATGCTTCTCCACTACTACTCAGACAGACATGGTCTGTGTCACATTGTACC
AGGTATCATTGAAGCTGTGGCCAAGGACTTCTTTGACACTGATGTCGCCATGAG
TATCCTGGATATGAACGAAGAGGTGGAAAGGACAGGGAAGAAGAACATGTTGTG
TTTCTGGTCGTGCAGAAGGCTCACAGACAGATAAGAGGAGCAAAGGCAAGCCGG
CCACAAGGCAGTGAGGACAGCCAGGCAGACCAGGAGGCTCTCCAGGGAACACTC
CTTCGGATGAAGGAGAGATATTTAAACATCCCTGTTTGCCCTGGGGAGAAATCT
CACTCAACTGCTGTGAGGGCATCGGTCCTTTTTGGAAAAGGGCCCCTCAGGGAC
ACCTTCCAGCCCGTCTATCCTGAGAGACTATGGGTCGAAGAGGAGGTGTTCTGT
GATGCTTTTCCTTTCCACATTGTCTTTGATGAAGCACTAAGGGTCAAGCAAGCT
GGAGTGAATATTCAGAAGTATGTCCCTGGAATCTTAACCCAGAAGTTTGCACTA
GATGAGTATTTTTCCATCATCCACCCTCAAGTTACTTTCAACATCTCCAGCATC
TGCAAGTTCATTAACAGTCAGTTTGTCTTGAAGACAAGAAAAGAAATGATGCCC
AAAGCAAGGAAGAGCCAGCCGATGCTCAAACTCCGGGGTCAGATGATCTGGATG
GAGTCTCTGAGGTGCATGATCTTCATGTGTGTTCCCCAAACGTCCGCAGCCTGC
AAGAGCTGGAAGAGAGCAAGATGCATCTTTCTGATATCGCTCCGCACGACACGA
CCAGGGATCTCATCCTCCTCAACCAGCAGAGGCTGGCAGAGATGGAGCTGTCCT
GCCAACTGGAAAAGAAGAAGGAGGAGTTGCGTGTCCTTTCCAATCACCTGGCCA
TCGAGAAGAAGAAGACAGAGACCTTGCTGTATGCCATGCTCCCTGAACATGTGC
CCAACCAACTCAAGGAGGCCAGAAAGGTGGCTCCAGGAGAATTTGAAACATCTA
CAATCCTTTTCAGCGATGTTGTGACATTTACCAACATCTGTGCAGCCTGTGAAC
CTATCCAAATCGTGAACATGCTGAATTCAATGTACTCCAAGTTTGACAGGTTAA
CCAGTGTCCATGATGTCTACAAAGTAGAAACAATAGCGGATGCTTACATGGTGG
TGCGTGGAGTACCAGTACCCCTTGAAAGCCATGCTCAAAGAGTCGCCAATTTTG
CTCTGGGGATGAGAATTTCTGCAAAAGAAGTGATGAATCCTGTCACTGGGGAAC
CTATCCAGATCAGAGTGGGAATCCACACTGGACCAGTCTTAGCAGGTGTTGTGG
GAGACAAGATGCCTCGGTACTGCTTGTTTGGTGACACTGTAAACACAGCCTCTA
GGATGGAAAGTCACGGGCTTCCCAGCAAAGTGCATCTGAGCCCCACAGCCCACA
GAGCCCTGAAAAACAAAGGGTTTGAAATTGTCAGGAGAGGCGAGATCGAAGTGA
AGGGGAAAGGAAAGATGACCACATACTTTCTGATCCAGAACCTGAATGCCACCG
AGGATGAGATAATGGGGCGACCTTCAGCCCCCCGCTGATGGGAAGGAAGTATGT
ACTCCCGGAAACCAAGTCAGGAAGTCCCCTGCTGTCCCGAGGAACACAGACCAT
CAGCAACAAGTCTACAAAGGAGACCCAGCAGACGCTTCTAATGAAGTCACACTT
GCTGGGAGCCCAGTGGCAGGGCGAAACTCCACAGATGCAGTCAATAACCAGCCA
TCACCAGATGAGACCAAGACAAGTGTCGTTGCTAGTGGCCCTGTGCTGTCTGCT
TTCTGTGTTGTGCTGTGA (SEQ ID NO: 21)
MYGFINTCLQSLVTEKFGEETWEKLKAPAEVQDVFMTYTVYDDIITIKLIQEACKV
LDVSMEAILKLFGEYFFKFCKMSGYDRMLRTLGGNLTEFIENLDALHSYLALSYQE
MNAPSFRVEEGADGAMLLHYYSDRHGLCHIVPGIIEAVAKDFFDTDVAMSILDMNE
EVERTGKKEHVVFLVVQKAHRQIRGAKASRPQGSEDSQADQEALQGTLLRMKERYL
NIPVCPGEKSHSTAVRASVLFGKGPLRDTFQPVYPERLWVEEEVFCDAFPFHIVFD
EALRVKQAGVNIQKYVPGILTQKFALDEYFSIIHPQVTFNISSICKFINSQFVLKT
RKEMMPKARKSQPMLKLRGQMIWMESLRCMIFMCSPNVRSLQELEESKMHLSDIAP
HDTTRDLILLNQQRLAEMELSCQLEKKKEELRVLSNHLAIEKKKTETLLYAMLPEH
VANQLKEGRKVAAGEFETCTILFSDVVTFTNICAACEPIQIVNMLNSMYSKFDRLT
SVHDVYKVETIGDAYMVVGGVPVPVESHAQRVANFALGMRISAKEVMNPVTGEPIQ
IRVGIHTGPVLAGVVGDKMPRYCLFGDTVNTASRMESHGLPSKVHLSPTAHRALKN
KGFEIVRRGEIEVKGKGKMTTYFLIQNLNATEDIMGRPSAPADGKEVCTPGNQVRK
SPAVPRNTDHQQQVYKGDPADASNEVTLAGSPVAGRNSTDAVNNQPSPDETKTSVV
ASGPVLSAFCVVL (SEQ ID NO: 22)

1-141. (canceled)

142. A method for increasing lymphocyte infiltration to a tumor in an individual comprising administering to the individual an effective amount of a trimeric H-NOX protein wherein the trimeric H-NOX protein comprises three monomers, wherein the monomers each comprise a T. tengcongensis H-NOX domain and a trimerization domain, wherein each of the three monomers is covalently bound to polyethylene glycol (PEG), wherein each T. tengcongensis H-NOX domain has the amino acid sequence of SEQ ID NO: 2 except for a L144F amino acid substitution in SEQ ID NO: 2, and wherein the trimerization domain is a foldon domain of bacteriophage T4 fibritin.

143. The method of claim 142, wherein the increase in lymphocyte infiltration to the tumor comprises an increase in infiltration of one or more of CD4 cells, CD8 cells, or NK cells.

144. The method of claim 142, wherein the increase in lymphocyte infiltration to the tumor is accompanied by inhibition of one or more of Treg cells, tumor associated macrophages or myeloid derived suppressor cells in the tumor.

145. The method of claim 142, wherein the increase in lymphocyte infiltration to the tumor is accompanied by an increase in MHC1 expression on the tumor cells.

146. The method of claim 142, wherein the C-terminus of the H-NOX domain is covalently linked to the trimerization domain.

147. The method of claim 142, wherein the H-NOX domain in each of the three monomers is linked via an amino acid linker to the foldon domain of T4 bacteriophage fibritin, wherein the amino acid linker is three, four, five, six, seven, eight, nine, or ten amino acids in length.

148. The method of claim 147, wherein the amino acid linker comprises Gly-Ser-Gly.

149. The method of claim 142, wherein each of the three monomers comprises three PEG molecules per monomer, wherein the molecular weight of each of the three PEG molecules is between 1 kDa and 10 kDa.

150. The method of claim 142, wherein each of the three monomers comprises the amino acid sequence of SEQ ID NO:8.

151. The method of claim 142, wherein the cancer is brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma, or squamous cell cancer.

152. The method of claim 142, wherein the administering of the trimeric H-NOX protein is intravenous.

153. The method of claim 142, wherein the administering of the trimeric H-NOX protein is by a bolus and/or by infusion.

154. The method of claim 142, wherein the trimeric H-NOX protein is administered with an immunotherapy.

155. The method of claim 154, wherein the immunotherapy is an anti-PD-1 therapy, an anti-PD-L1 therapy, and/or a dual PD-1/PD-L1 blockade therapy.

156. The method of claim 155, wherein the anti-PD-1 therapy is an anti-PD-1 antibody, the anti-PD-L1 therapy is an anti-PD-L1 antibody, and the dual PD-1/PD-L1 blockade therapy is an anti-PD-1 antibody and an anti-PD-L1 antibody.

157. The method of claim 142, further comprising administering a radiation therapy or chemotherapy to the individual.

158. The method of claim 156, wherein the immunotherapy is an anti-PD-1 antibody.

159. The method of claim 142, wherein the trimeric H-NOX protein is not encapsulated.

160. The method of claim 142, wherein the foldon domain comprises the amino acid sequence of SEQ ID NO: 4.

161. A method for modulating tumor immunity in an individual with a tumor comprising administering to the individual an effective amount of an H-NOX protein, wherein the trimeric H-NOX protein comprises three monomers, wherein the monomers each comprise a T. tengcongensis H-NOX domain and a trimerization domain, wherein each of the three monomers is covalently bound to polyethylene glycol (PEG), wherein each T. tengcongensis H-NOX domain has the amino acid sequence of SEQ ID NO: 2 except for a L144F amino acid substitution in SEQ ID NO: 2, and wherein the trimerization domain is a foldon domain of bacteriophage T4 fibritin.


SEQUENCE LISTING (30 sequences)

SEQ ID NO: 1 - DNA, 555 bp, Thermoanaerobacter tengcongensis
  source 1..555: mol_type = genomic DNA
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggcttatagagggtagttctaaatttttcaaggaagaaatttcagtggaagaggtcgaaagaggcgaaaaagatggcttttcaaggctaaaagtcaggataaaatttaaaaaccccgtttttgagtga

SEQ ID NO: 2 - protein, 184 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGLIEGSSKFFKEEISVEEVERGEKDGFSRLKVRIKFKNPVFE

SEQ ID NO: 3 - DNA, 81 bp, Thermoanaerobacter tengcongensis
  source 1..81: mol_type = genomic DNA
ggttatattcctgaagctccaagagatgggcaagcttacgttcgtaaagatggcgaatgggtattactttctaccttttta

SEQ ID NO: 4 - protein, 27 aa, Thermoanaerobacter tengcongensis
GYIPEAPRDGQAYVRKDGEWVLLSTFL

SEQ ID NO: 5 - DNA, 690 bp, Thermoanaerobacter tengcongensis
  source 1..690: mol_type = genomic DNA
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggtttatagagggtagttctaaatttttcaaggaagaaatttcagtggaagaggtcgaaagaggcgaaaaagatggcttttcaaggctaaaagtcaggataaaatttaaaaaccccgtttttgagtataagaaaaatctcgagggcagcggcggttatattcctgaagctccaagagatgggcaggcttacgttcgtaaagatggcgaatgggtattactttctacctttttaaggggtagtcaccatcaccatcaccattga

SEQ ID NO: 6 - protein, 225 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGFIEGKFFKEEIEEVERGEKDGFSRLKVRIKFKNPVFEYKKNLEGSGGYIPEAPRDGQAYVRKDGEWVLLSTFLRGSHHHHHH

SEQ ID NO: 7 - DNA, 663 bp, Thermoanaerobacter tengcongensis
  source 1..663: mol_type = genomic DNA
atgaaggggacaatcgtcgggacatggataaagaccctgagggacctttacgggaatgatgtggttgatgaatctttaaaaagtgtgggttgggaaccagatagggtaattacacctctggaggatattgatgacgatgaggttaggagaatttttgctaaggtgagtgaaaaaactggtaaaaatgtcaacgaaatatggagagaggtaggaaggcagaacataaaaactttcagcgaatggtttccctcctattttgcagggagaaggctagtgaattttttaatgatgatggatgaggtacacctacagcttaccaagatgataaaaggagccactcctccaaggcttattgcaaagcctgttgcaaaagatgccattgaaatggagtacgtttctaaaagaaagatgtacgattactttttagggtttatagagggtagttctaaatttttcaaggaagaaatttcagtggaagaggtcgaaagaggcgaaaaagatggcttttcaaggctaaaagtcaggataaaatttaaaaaccccgtttttgagtataagaaaaatctcgagggcagcggcggttatattcctgaagctccaagagatgggcaggcttacgttcgtaaagatggcgaatgggtattactttctacctttttatga

SEQ ID NO: 8 - protein, 220 aa, Thermoanaerobacter tengcongensis
MKGTIVGTWIKTLRDLYGNDVVDESLKSVGWEPDRVITPLEDIDDDEVRRIFAKVSEKTGKNVNEIWREVGRQNIKTFSEWFPSYFAGRRLVNFLMMMDEVHLQLTKMIKGATPPRLIAKPVAKDAIEMEYVSKRKMYDYFLGFIEGSSKFFKEEISVEEVERGEKDGFSRLKVRIKFKNPVFEYKKNLEGSGGYIPEAPRDGQAYVRKDGEWVLLSTFL

SEQ ID NO: 9 - DNA, 564 bp, Legionella pneumophila
  source 1..564: mol_type = genomic DNA
atgatgtctatgaaaggaatcatattcaacgaatttctcaattttgtagaaaaaagtgaatcctacaccctggtagatcaaattattatggatagtcatttgaagtcccatggtgcctacacgtctatcggtacatactctcccaaagaattatttcaattggttaaagcgcttgctatgaaaaatggcaaaccaacatcagtgattttacaagaatatggtgagtatttgtttgaggtttttgcaaaaaaatatcctcaatttttcagggaaaaaaagtcggtgtttcaatttttggaagcgcttgaaacacatattcatttcgaagtgaaaaaattgtatgactatactgaactaccccattttgaatgccaatatcacagtcaaaatcaaatggaaatgatttacacttcttcgcgtcctttggccgattttgcggaaggtttaataaaaggttgtattaaatatcataaagaaaacatgactattgttcgtgaaaatctgcctgcaaaaacaggctttaaggtaagatttgtattaacaaaaggcgatcctgatgagtga

SEQ ID NO: 10 - protein, 187 aa, Legionella pneumophila
MMSMKGIIFNEFLNFVEKSESYTLVDQIIMDSHLKSHGAYTSIGTYSPKELFQLVKALAMKNGKPTSVILQEYGEYLFEVFAKKYPQFFREKKSVFQFLEALETHIHFEVKKLYDYTELPHFECQYHSQNQMEMIYTSSRPLADFAEGLIKGCIKYHKENMTIVRENLPAKTGFKVRFVLTKGDPDE

SEQ ID NO: 11 - DNA, 543 bp, Legionella pneumophila
  source 1..543: mol_type = genomic DNA
atgaaaggtatcgtttttacctccttaaatgacatgattatagaacaatttggcatagaaacctgggaccaactcgtatcctcactagaccttccaagtggtggaagttatacagcaggcggcacttactcggatacagaatttcagcaattgattaaggccattgcgaagaggaccaatcagcacgcttctgtttttttagaggcctttggtgaatacatgtttcctatcttatcgagtaagtgcgcaatttttttaaaaaaggacatgacattaaaagaatttttaaaaagcattgatggaacaattcatgtggaagtagaaaagttatacccagatgaaacattacctaccattagctatgaagagcctgctgcaaaccaattggttatggtgtatcgatcgcatagaagactctgtcattttgcaatggggctcatccagggagcagcgcaacattttaaaaagaaaattaccattaagcagactcactgcatgttaaaaaaagatgatcattgtcgtttggagattacctttgagtga

SEQ ID NO: 12 - protein, 180 aa, Legionella pneumophila
MKGIVFTSLNDMIIEQFGIETWDQLVSSLDLPSGGSYTAGGTYSDTEFQQLIKAIAKRTNQHASVFLEAFGEYMFPILSSKCAIFLKKDMTLKEFLKSIDGTIHVEVEKLYPDETLPTISYEEPAANQLVMVYRSHRRLCHFAMGLIQGAAQHFKKKITIKQTHCMLKKDDHCRLEITFE

SEQ ID NO: 13 - DNA, 1158 bp, Homo sapiens
  source 1..1158: mol_type = genomic DNA
atgtacggatttgtgaatcacgccctggagttgctggtgatccgcaattacggccccgaggtgtgggaagacatcaaaaaagaggcacagttagatgaagaaggacagtttcttgtcagaataatatatgatgactccaaaacttatgatttggttgctgctgcaagcaaagtcctcaatctcaatgctggagaaatcctccaaatgtttgggaagatgtttttcgtcttttgccaagaatctggttatgatacaatcttgcgtgtcctgggctctaatgtcagagaatttctacagaaccttgatgctctgcacgaccaccttgctaccatctacccaggaatgcgtgcaccttcctttaggtgcactgatgcagaaaagggcaaaggactcattttgcactactactcagagagagaaggacttcaggatattgtcattggaatcatcaaaacagtggcacaacaaatccatggcactgaaatagacatgaaggttattcagcaaagaaatgaagaatgtgatcatactcaatttttaattgaagaaaaagagtcaaaagaagaggatttttatgaagatcttgacagatttgaagaaaatggtacccaggaatcacgcatcagcccatatacattctgcaaagcttttccttttcatataatatttgaccgggacctagtggtcactcagtgtggcaatgctatatacagagttctcccccagctccagcctgggaattgcagccttctgtctgtcttctcgctggttcgtcctcatattgatattagtttccatgggatcctttctcacatcaatactgtttttgtattgagaagcaaggaaggattgttggatgtggagaaattagaatgtgaggatgaactgactgggactgagatcagctgcttacgtctcaagggtcaaatgatctacttacctgaagcagatagcatactttttctatgttcaccaagtgtcatgaacctggacgatttgacaaggagagggctgtatctaagtgacatccctctgcatgatgccacgcgcgatcttgttcttttgggagaacaatttagagaggaatacaaactcacccaagaactggaaatcctcactgacaggctacagctcacgttaagagccctggaagattga

SEQ ID NO: 14 - protein, 385 aa, Homo sapiens
MYGFVNHALELLVIRNYGPEVWEDIKKEAQLDEEGQFLVRIIYDDSKTYDLVAAASKVLNLNAGEILQMFGKMFFVFCQESGYDTILRVLGSNVREFLQNLDALHDHLATIYPGMRAPSFRCTDAEKGKGLILHYYSEREGLQDIVIGIIKTVAQQIHGTEIDMKVIQQRNEECDHTQFLIEEKESKEEDFYEDLDRFEENGTQESRISPYTFCKAFPFHIIFDRDLVVTQCGNAIYRVLPQLQPGNCSLLSVFSLVRPHIDISFHGILSHINTVFVLRSKEGLLDVEKLECEDELTGTEISCLRLKGQMIYLPEADSILFLCSPSVMNLDDLTRRGLYLSDIPLHDATRDLVLLGEQFREEYKLTQELEILTDRLQLTLRALED

SEQ ID NO: 15 - DNA, 651 bp, Homo sapiens
  source 1..651: mol_type = genomic DNA
atgtatggattcatcaacacctgcctgcagtctcttgtgacagagaaatttggtgaggagacatgggagaagctgaaggctcctgcagaagtgcaagatgtcttcatgacctacaccgtgtatgatgacatcatcaccattaagctcatccaagaagcctgcaaggttctggatgtgtccatggaagccattctgaagctctttggcgaatacttctttaagttctgtaagatgtctggctatgacaggatgctgcggacacttggaggaaatctcaccgagtttattgaaaacctagatgcactccacagttacctggcactgtcctatcaggaaatgaacgcaccatcctttcgagtggaggaaggagctgacggggcgatgcttctccactactactcagacagacatggtctgtgtcacattgtaccaggtatcattgaagctgtggccaaggacttctttgacactgatgtggccatgagtatcctggatatgaacgaagaggtggaaaggacagggaagaaagaacatgttgtgtttctggtcgtgcagaaggctcacagacagataagaggagcaaaggcaagccggccacaaggcagtgaggacagccaggcagaccaggaggctctccagggaacactcctt

SEQ ID NO: 16 - protein, 217 aa, Homo sapiens
MYGFINTCLQSLVTEKFGEETWEKLKAPAEVQDVFMTYTVYDDIITIKLIQEACKVLDVSMEAILKLFGEYFFKFCKMSGYDRMLRTLGGNLTEFIENLDALHSYLALSYQEMNAPSFRVEEGADGAMLLHYYSDRHGLCHIVPGIIEAVAKDFFDTDVAMSILDMNEEVERTGKKEHVVFLVVQKAHRQIRGAKASRPQGSEDSQADQEALQGTLL

SEQ ID NO: 17 - DNA, 1158 bp, Rattus norvegicus
  source 1..1158: mol_type = genomic DNA
atgtacggttttgtgaaccatgccctggagctgctggtgatccgcaattacggtcccgaggtgtgggaagacatcaaaaaagaggcgcagctggatgaagaaggccagtttcttgtgagaataatctacgatgattccaaaacctatgacttggtggctgctgcgagcaaagtcctcaacctcaatgctggtgaaatcctgcagatgtttgggaagatgtttttcgtcttctgtcaagagtctggctatgataccatcttgcgtgtcctgggatctaatgtcagggagtttttgcagaacctcgacgccctgcacgaccacctcgccaccatctacccagggatgcgcgcaccttccttccggtgcaccgatgcagaaaaaggcaaagggctcattctgcactactactcggaaagagaggggcttcaggacattgtgatcgggattatcaagactgtagctcaacagatccatggcactgagatagacatgaaggttattcagcaaagaagtgaagaatgtgatcatacccaatttttaattgaagaaaaagaatcaaaagaagaggatttttatgaagatctggacaggtttgaagagaacggtacccaggactcccgtatcagcccgtacaccttctgcaaagcgtttccttttcacatcatatttgaccgggacctagtagtcacgcagtgtggaaatgctatctacagagtgctcccccagctccagcctgggaagtgcagccttctgtctgtcttctctctggtccgccctcatattgacatcagtttccacgggattctttcacacatcaataccgtctttgtactgagaagcaaggaagggttgctggatgttgagaaacttgaatgtgaggatgaactgactggggcagagattagctgcctccgtctcaaaggccaaatgatctatttaccggaagcagatagcatcctcttcctctgttcaccaagtgtgatgaacttggatgacctaacaagaagaggcctgtacctgagtgacatccctctccatgatgctacacgagacctggtccttttgggagaacagttccgggaggagtacaaactgacacaagagctggaaatcctcacagacaggctgcagctcacactgagggctttggaggattga

SEQ ID NO: 18 - protein, 385 aa, Rattus norvegicus
MYGFVNHALELLVIRNYGPEVWEDIKKEAQLDEEGQFLVRIIYDDSKTYDLVAAASKVLNLNAGEILQMFGKMFFVFCQESGYDTILRVLGSNVREFLQNLDALHDHLATIYPGMRAPSFRCTDAEKGKGLILHYYSEREGLQDIVIGIIKTVAQQIHGTEIDMKVIQQRSEECDHTQFLIEEKESKEEDFYEDLDRFEENGTQDSRISPYTFCKAFPFHIIFDRDLVVTQCGNAIYRVLPQLQPGKCSLLSVFSLVRPHIDISFHGILSHINTVFVLRSKEGLLDVEKLECEDELTGAEISCLRLKGQMIYLPEADSILFLCSPSVMNLDDLTRRGLYLSDIPLHDATRDLVLLGEQFREEYKLTQELEILTDRLQLTLRALED

SEQ ID NO: 19 - DNA, 1158 bp, Rattus norvegicus; identical to SEQ ID NO: 17
  source 1..1158: mol_type = genomic DNA

SEQ ID NO: 20 - protein, 385 aa, Rattus norvegicus; identical to SEQ ID NO: 18

SEQ ID NO: 21 - DNA, 2229 bp, Rattus norvegicus
  source 1..2229: mol_type = genomic DNA
atgtatggattcatcaacacctgcctgcagtctcttgtgacagagaaatttggtgaggagacatgggagaagctgaaggctcctgcagaagtgcaagatgtcttcatgacctacaccgtgtatgatgacatcatcaccattaagctcatccaagaagcctgcaaggttctggatgtgtccatggaagccattctgaagctctttggcgaatacttctttaagttctgtaagatgtctggctatgacaggatgctgcggacacttggaggaaatctcaccgagtttattgaaaacctagatgcactccacagttacctggcactgtcctatcaggaaatgaacgcaccatcctttcgagtggaggaaggagctgacggggcgatgcttctccactactactcagacagacatggtctgtgtcacattgtaccaggtatcattgaagctgtggccaaggacttctttgacactgatgtggccatgagtatcctggatatgaacgaagaggtggaaaggacagggaagaaagaacatgttgtgtttctggtcgtgcagaaggctcacagacagataagaggagcaaaggcaagccggccacaaggcagtgaggacagccaggcagaccaggaggctctccagggaacactccttcggatgaaggagagatatttaaacatccctgtttgccctggggagaaatctcactcaactgctgtgagggcatcggtcctttttggaaaagggcccctcagggacaccttccagcccgtctatcctgagagactatgggtcgaagaggaggtgttctgtgatgcttttcctttccacattgtctttgatgaagcactaagggtcaagcaagctggagtgaatattcagaagtatgtccctggaatcttaacccagaagtttgcactagatgagtatttttccatcatccaccctcaagttactttcaacatctccagcatctgcaagttcattaacagtcagtttgtcttgaagacaagaaaagaaatgatgcccaaagcaaggaagagccagccgatgctcaaactccggggtcagatgatctggatggagtctctgaggtgcatgatcttcatgtgttccccaaacgtccgcagcctgcaagagctggaagagagcaagatgcatctttctgatatcgctccgcacgacacgaccagggatctcatcctcctcaaccagcagaggctggcagagatggagctgtcctgccaactggaaaagaagaaggaggagttgcgtgtcctttccaatcacctggccatcgagaagaagaagacagagaccttgctgtatgccatgctgcctgaacatgtggccaaccaactcaaggagggcagaaaggtggctgcaggagaatttgaaacatgtacaatccttttcagcgatgttgtgacatttaccaacatctgtgcagcctgtgaacctatccaaatcgtgaacatgctgaattcaatgtactccaagtttgacaggttaaccagtgtccatgatgtctacaaagtagaaacaataggggatgcttacatggtggtgggtggagtaccagtacccgttgaaagccatgctcaaagagtcgccaattttgctctggggatgagaatttctgcaaaagaagtgatgaatcctgtcactggggaacctatccagatcagagtgggaatccacactggaccagtcttagcaggtgttgtgggagacaagatgcctcggtactgcttgtttggtgacactgtaaacacagcctctaggatggaaagtcacgggcttcccagcaaagtgcatctgagccccacagcccacagagccctgaaaaacaaagggtttgaaattgtcaggagaggcgagatcgaagtgaaggggaaaggaaagatgaccacatactttctgatccagaacctgaatgccaccgaggatgagataatggggcgaccttcagcccccgctgatgggaaggaagtatgtactcccggaaaccaagtcaggaagtcccctgctgtcccgaggaacacagaccatcagcaacaagtctacaaaggagacccagcagacgcttctaatgaagtcacacttgctgggagcccagtggcagggcgaaactccacagatgcagtcaataaccagccatcaccagatgagaccaagacaagtgtcgttgctagtggccctgtgctgtctgctttctgtgttgtgctgtga

SEQ ID NO: 22 - protein, 742 aa, Rattus norvegicus
MYGFINTCLQSLVTEKFGEETWEKLKAPAEVQDVFMTYTVYDDIITIKLIQEACKVLDVSMEAILKLFGEYFFKFCKMSGYDRMLRTLGGNLTEFIENLDALHSYLALSYQEMNAPSFRVEEGADGAMLLHYYSDRHGLCHIVPGIIEAVAKDFFDTDVAMSILDMNEEVERTGKKEHVVFLVVQKAHRQIRGAKASRPQGSEDSQADQEALQGTLLRMKERYLNIPVCPGEKSHSTAVRASVLFGKGPLRDTFQPVYPERLWVEEEVFCDAFPFHIVFDEALRVKQAGVNIQKYVPGILTQKFALDEYFSIIHPQVTFNISSICKFINSQFVLKTRKEMMPKARKSQPMLKLRGQMIWMESLRCMIFMCSPNVRSLQELEESKMHLSDIAPHDTTRDLILLNQQRLAEMELSCQLEKKKEELRVLSNHLAIEKKKTETLLYAMLPEHVANQLKEGRKVAAGEFETCTILFSDVVTFTNICAACEPIQIVNMLNSMYSKFDRLTSVHDVYKVETIGDAYMVVGGVPVPVESHAQRVANFALGMRISAKEVMNPVTGEPIQIRVGIHTGPVLAGVVGDKMPRYCLFGDTVNTASRMESHGLPSKVHLSPTAHRALKNKGFEIVRRGEIEVKGKGKMTTYFLIQNLNATEDEIMGRPSAPADGKEVCTPGNQVRKSPAVPRNTDHQQQVYKGDPADASNEVTLAGSPVAGRNSTDAVNNQPSPDETKTSVVASGPVLSAFCVVL

SEQ ID NO: 23 - protein, 19 aa, synthetic construct
CAATCGAGACCCTGGTGGA

SEQ ID NO: 24 - protein, 18 aa, synthetic construct
GCACACACTCCAGGCCCT

SEQ ID NO: 25 - protein, 19 aa, synthetic construct
CAACCAGACATGGGTCCAC

SEQ ID NO: 26 - protein, 21 aa, synthetic construct
GTTAACGAAAAGGCCCACAGA

SEQ ID NO: 27 - protein, 21 aa, synthetic construct
GTTGTGGATCCAGTCACCTCT

SEQ ID NO: 28 - protein, 21 aa, synthetic construct
GATTCTCAGTGTGCTGGTCAC

SEQ ID NO: 29 - protein, 23 aa, synthetic construct
CAAGGAGGAAGCTTATCTATGAA

SEQ ID NO: 30 - protein, 22 aa, synthetic construct
ATTTGACGAAGGCGAAGAAGCT